(19) United States
(12) Patent Application Publication
(10) Pub. No.: US 2026/0048036 A1
(43) Pub. Date: Feb. 19, 2026

(54) CRYSTALLINE FORMS OF A GLUCOSYLCERAMIDE SYNTHASE INHIBITOR AND USES THEREOF

(71) Applicant: ACELINK THERAPEUTICS, INC., Newark, CA (US)

(72) Inventors: Jianhong ZHENG, Shanghai (CN); Xiaoyang WANG, Shanghai (CN); Hongyan HE, Shanghai (CN)

(21) Appl. No.: 19/104,233

(22) PCT Filed: Aug. 10, 2023
(86) PCT No.: PCT/CN2023/112299

§ 371 Date: Feb. 17, 2025

(30) Foreign Application Priority Data
Aug. 18, 2022 (WO) PCT/CN2022/113204

(51) Int. Cl. A61K 31/40; A61P 3/00; C07D 207/06; C12N 9/99

(52) U.S. Cl. CPC A61K 31/40; A61P 3/00; C07D 207/06; C12N 9/99

(57) ABSTRACT

The present disclosure relates to crystalline forms of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide, the method of preparing the forms, pharmaceutical compositions comprising the forms, and uses thereof.

### FIELD OF THE INVENTION

The present disclosure generally relates to crystalline forms of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide, the method of preparing the forms, pharmaceutical compositions comprising the forms, and uses thereof.

### BACKGROUND

Glucosylceramide synthase (GCS) is a key enzyme which catalyzes the initial glycosylation step in the biosynthesis of glucosylceramide-based glycosphingolipids (GSLs) namely via the transfer of glucose from UDP-glucose (UDP-Gle) to ceramide to form glucosylceramide. GCS inhibitors have been proposed for the treatment of a variety of diseases (see, for example, WO2005068426).

As described in the PCT Publication No. WO2015042397A1, (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide (hereinafter referred to as “Compound 1”) is a GCS inhibitor, which can be used for preventing or treating GCS associated disease, disorder or condition. The PCT Publication No. WO2015042397A1 is incorporated herein by reference in its entirety. The structure of Compound 1 is shown as follows:

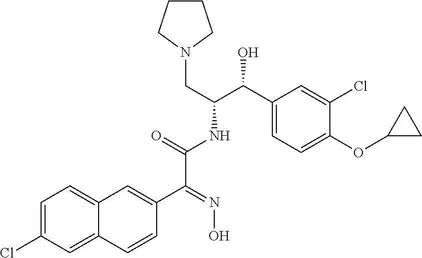 [SMILES: O=C(N[C@H](CN1CCCC1)[C@H](O)c1ccc(OC2CC2)c(Cl)c1)/C(=N/O)c1ccc2cc(Cl)ccc2c1]

A compound can exist in one or more crystalline forms. Crystalline forms of a drug substance can have different physical properties, including melting point, solubility, dissolution rate, optical and mechanical properties, vapor pressure, hygroscopicity, particle shape, density, and flowability, etc. These properties can have a direct effect on the ability to process and/or manufacture a compound as a drug product. Crystalline forms can also exhibit different stabilities and bioavailability. The most stable crystalline form of a drug product is often chosen during drug development based on the minimal potential for conversion to another crystalline form and on its greater chemical stability. To ensure the quality, safety, and efficacy of a drug product, it is important to choose a crystalline form that is stable, is manufactured reproducibly, and has favorable physicochemical properties.

Therefore, there is still a need for a stable form of Compound 1 with advantageable chemical and physical properties.

### SUMMARY OF INVENTION

In one aspect, the present disclosure provides Compound 1, and various crystalline forms of its anhydrate, hydrate, solvates and salts.

In some embodiments, the present disclosure provides a Crystalline Form A of Compound 1, wherein the Crystalline Form A is characterized by an X-ray powder diffraction (XRPD) pattern comprising one or more peaks selected from the group consisting of about 18.883°, about 10.889°, and about 21.309° 2θ.

In some embodiments, the present disclosure provides a Crystalline Form B of Compound 1, wherein the Crystalline Form B is characterized by an XRPD pattern comprising one or more peaks selected from the group consisting of about 6.244°, about 18.679°, and about 12.445° 2θ.

In some embodiments, the present disclosure provides a Crystalline Form C of Compound 1, wherein the Crystalline Form C is characterized by an XRPD pattern comprising one or more peaks selected from the group consisting of about 6.656°, about 19.994°, and about 26.732° 2θ.

In some embodiments, the present disclosure provides a Crystalline Form D of Compound 1, wherein the Crystalline Form D is characterized by an XRPD pattern comprising one or more peaks selected from the group consisting of about 16.956°, about 5.883°, and about 22.181° 2θ.

In some embodiments, the present disclosure provides a Crystalline Form E of Compound 1, wherein the Crystalline Form E is characterized by an XRPD pattern comprising one or more peaks selected from the group consisting of about 18.089°, about 26.315°, and about 19.775° 2θ.

In some embodiments, the present disclosure provides a Crystalline Form A of L-malate Salt of Compound 1, wherein the Crystalline Form A of L-malate Salt is characterized by an XRPD pattern comprising one or more peaks selected from the group consisting of about 19.349°, about 22.304°, and about 20.078° 2θ.

In some embodiments, the present disclosure provides a Crystalline Form A of L-tartrate Salt of Compound 1, wherein the Crystalline Form A of L-tartrate Salt is characterized by an XRPD pattern comprising one or more peaks selected from the group consisting of about 18.564°, about 21.685°, and about 18.774° 2θ.

In another aspect, the present disclosure provides a pharmaceutical composition comprising the crystalline forms of Compound 1 provided herein.

In another aspect, the present disclosure provides a method for preparing the crystalline forms of Compound 1, or the pharmaceutical composition provided herein.

In another aspect, the present disclosure provides use of the crystalline forms of Compound 1 or the pharmaceutical composition provided herein in the manufacture of a medicament for (a) preventing or treating a disease, disorder or condition that is associated with an abnormal activity/level of the enzyme GCS; (b) preventing or treating a GCS-mediated disease, disorder or condition; (c) inhibiting an activity/level of the enzyme GCS; or (d) non-therapeutically inhibiting an activity/level of the enzyme GCS in vitro.

In another aspect, the present disclosure provides a method of inhibiting GCS activity, comprising administering to a subject in need thereof an effective amount of the crystalline forms of Compound 1 or the pharmaceutical composition provided herein.

### BRIEF DESCRIPTION OF THE DRAWINGS

FIG. 1 shows the XRPD pattern of Crystalline Form A of Compound 1.

FIG. 2 shows the TGA curve of Crystalline Form A of Compound 1.

FIG. 3 shows the DSC curve of Crystalline Form A of Compound 1.

FIG. 4 shows the ¹H-NMR spectrum of Crystalline Form A of Compound 1.

FIG. 5 shows the XRPD pattern of Crystalline Form B of Compound 1.

FIG. 6 shows the TGA curve of Crystalline Form B of Compound 1.

FIG. 7 shows the DSC curve of Crystalline Form B of Compound 1.

FIG. 8 shows the ¹H-NMR spectrum of Crystalline Form B of Compound 1.

FIG. 9 shows the XRPD pattern of Crystalline Form C of Compound 1.

FIG. 10 shows the TGA curve of Crystalline Form C of Compound 1.

FIG. 11 shows the DSC curve of Crystalline Form C of Compound 1.

FIG. 12 shows the ¹H-NMR spectrum of Crystalline Form C of Compound 1.

FIG. 13 shows the XRPD pattern of Crystalline Form D of Compound 1.

FIG. 14 shows the TGA curve of Crystalline Form D of Compound 1.

FIG. 15 shows the DSC curve of Crystalline Form D of Compound 1.

FIG. 16 shows the ¹H-NMR spectrum of Crystalline Form D of Compound 1.

FIG. 17 shows the XRPD pattern of Crystalline Form E of Compound 1.

FIG. 18 shows the XRPD pattern of Crystalline Form A of L-malate Salt of Compound 1.

FIG. 19 shows the TGA curve of Crystalline Form A of L-malate Salt of Compound 1.

FIG. 20 shows the DSC curve of Crystalline Form A of L-malate Salt of Compound 1.

FIG. 21 shows the ¹H-NMR spectrum of Crystalline Form A of L-malate Salt of Compound 1.

FIG. 22 shows the XRPD pattern of Crystalline Form A of L-tartrate Salt of Compound 1.

FIG. 23 shows the TGA curve of Crystalline Form A of L-tartrate Salt of Compound 1.

FIG. 24 shows the DSC curve of Crystalline Form A of L-tartrate Salt of Compound 1.

FIG. 25 shows the ¹H-NMR spectrum of Crystalline Form A of L-tartrate Salt of Compound 1.

FIG. 26 shows the DVS isotherm plot of Crystalline Form A of Compound 1 at 25° C.

FIG. 27 shows the XRPD overlay of Crystalline Form A of Compound 1 before and after DVS.

FIG. 28 shows the DVS isotherm plot of Crystalline Form A of L-malate Salt of Compound 1 at 25° C.

FIG. 29 shows the XRPD overlay of Crystalline Form A of L-malate Salt of Compound 1 before and after DVS.

FIG. 30 shows the DVS isotherm plot of Crystalline Form A of L-tartrate Salt of Compound 1 at 25° C.

FIG. 31 shows the XRPD overlay of Crystalline Form A of L-tartrate Salt of Compound 1 before and after DVS.

FIG. 32A-C show the results of GCS inhibitors Eliglustat (FIG. 32A), Venglustat (FIG. 32B) and Crystalline Form A of Compound 1 (FIG. 32C) effect test with MDCK cell lysate.

FIG. 33A-D show the results of GCS inhibitors Eliglustat (FIG. 33A), Venglustat (FIG. 33B), Crystalline Form A of Compound 1 (FIG. 33C) and geometric isomer of Compound 1 (FIG. 33D) effect test with live K-562 cells.

FIG. 34 shows the results of GCS activity detection on L6 cell lysate.

### DETAILED DESCRIPTION OF THE INVENTION

Throughout the present disclosure, the articles “a,” “an,” and “the” are used herein to refer to one or to more than one (i.e., to at least one) of the grammatical object of the article. By way of example, “a crystalline form” means one crystalline form or more than one crystalline form.

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entireties.

### I. Definitions

Unless otherwise specified, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

The terms “effective amount” or “therapeutically effective amount,” as used herein, refer to a sufficient amount of an agent or a compound being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The terms “excipient” or “pharmaceutically acceptable excipient,” as used herein, refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” as used herein refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt is not specifically limited as far as it can be used in medicaments. Examples of a salt that the compound of the present invention forms with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

With regard to the specific crystal form of Compound 1, in certain embodiments, the term “substantially pure” as used herein refers to that the specific crystalline form of Compound 1 contains less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1% by weight of the other substances, including the other crystal forms and/or impurities. For example, the impurities may include by-products, reaction starting materials, reagents from chemical reactions, contaminants, degradation products, water or solvents, etc. In some embodiments, if a specific crystalline form of Compound 1 is substantially pure, it means the crystalline form of Compound 1 contains less than 10%, less than 9%, less than 8%, less than 7%, less than 6%, less than 5%, less than 4%, less than 3%, less than 2%, less than 1% by weight of the other substances.

The term “polymorphs” as used herein refers to different crystal structures (in solvated or unsolvated forms) that a compound can crystallize. For example, Compound 1 of the present disclosure can be crystallized to form different crystal structures, that is, polymorphs.

The term “X-ray powder diffractogram pattern” or “XRPD pattern” as used herein refers to an x-y graph with diffraction angle (i.e., 20) on the x-axis and intensity on the y-axis. The peaks within this pattern may be used to characterize a crystalline solid form. As with any data measurement, there is variability in XRPD data. The data are often represented solely by the diffraction angle of the peaks rather than including the intensity of the peaks because peak intensity can be particularly sensitive to sample preparation (for example, particle size, moisture content, solvent content, and preferred orientation effects influence the sensitivity), so samples of the same material prepared under different conditions may yield slightly different patterns; this variability is usually greater than the variability in diffraction angles. Diffraction angle variability may also be sensitive to sample preparation. Other sources of variability come from instrument parameters and processing of the raw X-ray data: different X-ray instruments operate using different parameters and these may lead to slightly different XRPD patterns from the same solid form, and similarly different software packages process X-ray data differently and this also leads to variability. These and other sources of variability are known to those of ordinary skill in the pharmaceutical arts. Due to such sources of variability, it is usual to assign a variability of ±0.3° 2θ to diffraction angles in XRPD patterns. In the present disclosure, for example, when referring to diffraction angle °2θ, the term “about” means a variability of ±0.3° 2θ can be assigned to the mentioned value, for example, the variability can be −0.3°, −0.29°, −0.28°, −0.27°, −0.26°, −0.25°, −0.24°, −0.23°, −0.22°, −0.21°, −0.2°, −0.19°, −0.18°, −0.17°, −0.16°, −0.15°, −0.14°, −0.13°, −0.12°, −0.11°, −0.1°, −0.09°, −0.08°, −0.07°, −0.06°, −0.05°, −0.04°, −0.03°, −0.02°, −0.01°, 0.00°, 0.01°, 0.02°, 0.03°, 0.04°, 0.05°, 0.06°, 0.07°, 0.08°, 0.09°, 0.1°, 0.11°, 0.12°, 0.13°, 0.14°, 0.15°, 0.16°, 0.17°, 0.18°, 0.19°, 0.2°, 0.21°, 0.22°, 0.23°, 0.24°, 0.25°, 0.26°, 0.27°, 0.28°, 0.29°, or 0.3°, preferably ±0.2°.

The term “differential scanning calorimetry” or “DSC” as used herein refers to the measurement of the temperature difference and heat flow difference between the sample and the reference material during the temperature rise or constant temperature process of the sample to characterize all physical changes and chemical changes related to the thermal effect, so as to obtain the phase change information of the sample.

In the present disclosure, when referring to patterns (for example, XRPD patterns, DSC curves, TGA curves, HPLC patterns, ¹H NMR patterns, DVS patterns, etc.), the term “substantially similar to” or “substantially as shown in” means that the basic feature information or main feature information (for example, main peak position, intensity, etc.) of the pattern is consistent with the information depicted in the mentioned pattern, and it is not required that all the features of the pattern are exactly the same as those described in the mentioned pattern.

### II. Crystalline Form A of Compound 1

In one aspect, the present disclosure provides a Crystalline Form A of Compound 1. In some embodiments, the present disclosure provides a substantially pure Crystalline Form A of Compound 1. In some embodiments, Compound 1 is in an unsolvated crystalline form, for example, the Crystalline Form A of Compound 1 is an anhydrate.

In some embodiments, the XRPD pattern of Crystalline Form A of Compound 1 comprises one or more (e.g., two, three) peaks selected from the group consisting of about 18.883°, about 10.889°, and about 21.309° 2θ. For example, the XRPD pattern of Crystalline Form A of Compound 1 comprises a peak at about 18.883° 2θ. For another example, the XRPD pattern of Crystalline Form A of Compound 1 comprises a peak at about 10.889° 2θ. For yet another example, the XRPD pattern of Crystalline Form A of Compound 1 comprises a peak at about 21.309° 2θ. For yet another example, the XRPD pattern of Crystalline Form A of Compound 1 comprises two peaks at about 18.883° and about 10.889° 2θ. For yet another example, the XRPD pattern of Crystalline Form A of Compound 1 comprises two peaks at about 10.889° and about 21.309° 2θ. For yet another example, the XRPD pattern of Crystalline Form A of Compound 1 comprises two peaks at about 18.883° and about 21.309° 2θ. In some embodiments, the XRPD pattern of Crystalline Form A of Compound 1 comprises all the peaks selected from the group consisting of about 18.883°, about 10.889°, and about 21.309° 2θ.

In some embodiments, the XRPD pattern of Crystalline Form A of Compound 1 further comprises one or more (e.g., 2, 3, 4, 5, 6, 7, 8, 9) peaks selected from the group consisting of about 14.974°, about 24.160°, about 18.663°, about 17.109°, about 7.518°, about 20.950°, about 19.602°, about 27.784°, and about 28.281° 2θ. For example, the XRPD pattern of Crystalline Form A of Compound 1 further comprises a peak at about 14.974°, about 24.160°, about 18.663°, about 17.109°, about 7.518°, about 20.950°, about 19.602°, about 27.784°, or about 28.281° 2θ. In some embodiments, the XRPD pattern of Crystalline Form A of Compound 1 further comprises all the peaks selected from the group consisting of about 14.974°, about 24.160°, about 18.663°, about 17.109°, about 7.518°, about 20.950°, about 19.602°, about 27.784°, and about 28.281° 2θ.

In some embodiments, the XRPD pattern of Crystalline Form A of Compound 1 further comprises one or more (e.g., 2, 3, 4, 5, 6, 7, 8, 9, 10, 11, 12, 13, 14, 15, 16, 17) peaks selected from the group consisting of about 17.494°, about 14.743°, about 22.386°, about 31.979°, about 21.759°, about 25.017°, about 30.623°, about 10.301°, about 26.760°, about 21.536°, about 19.337°, about 30.103°, about 33.310°, about 31.467°, about 23.309°, about 25.781°, and about 27.362° 2θ. For example, the XRPD pattern of Crystalline Form A of Compound 1 further comprises a peak at about 17.494°, about 14.743°, about 22.386°, about 31.979°, about 21.759°, about 25.017°, about 30.623°, about 10.301°, about 26.760°, about 21.536°, about 19.337°, about 30.103°, about 33.310°, about 31.467°, about 23.309°, about 25.781°, or about 27.362° 2θ. For another example, the XRPD pattern of Crystalline Form A of Compound 1 further comprises all the peaks selected from the group consisting of about 17.494°, about 14.743°, about 22.386°, about 31.979°, about 21.759°, about 25.017°, about 30.623°, about 10.301°, about 26.760°, about 21.536°, about 19.337°, about 30.103°, about 33.310°, about 31.467°, about 23.309°, about 25.781°, and about 27.362° 2θ.

In some embodiments, the XRPD pattern of Crystalline Form A of Compound 1 comprises all the peaks selected from the group consisting of:

| Angle (°2θ) | Angle (°2θ) | Angle (°2θ) | Angle (°2θ) |
|---|---|---|---|
| 18.883 | 14.743 | 23.309 | 28.891 |
| 10.889 | 22.386 | 25.781 | 34.526 |
| 21.309 | 31.979 | 27.362 | 17.884 |
| 14.974 | 21.759 | 35.751 | 13.697 |
| 24.160 | 25.017 | 23.835 | 32.877 |
| 18.663 | 30.623 | 23.601 | 36.134 |
| 17.109 | 10.301 | 37.946 | 11.644 |
| 7.518 | 26.760 | 33.960 | 37.542 |
| 20.950 | 21.536 | 39.178 | 16.364 |
| 19.602 | 19.337 | 10.569 | 14.156 |
| 27.784 | 30.103 | 36.533 | 35.228 |
| 28.281 | 33.310 | 26.283 | |
| 17.494 | 31.467 | 29.254 | |

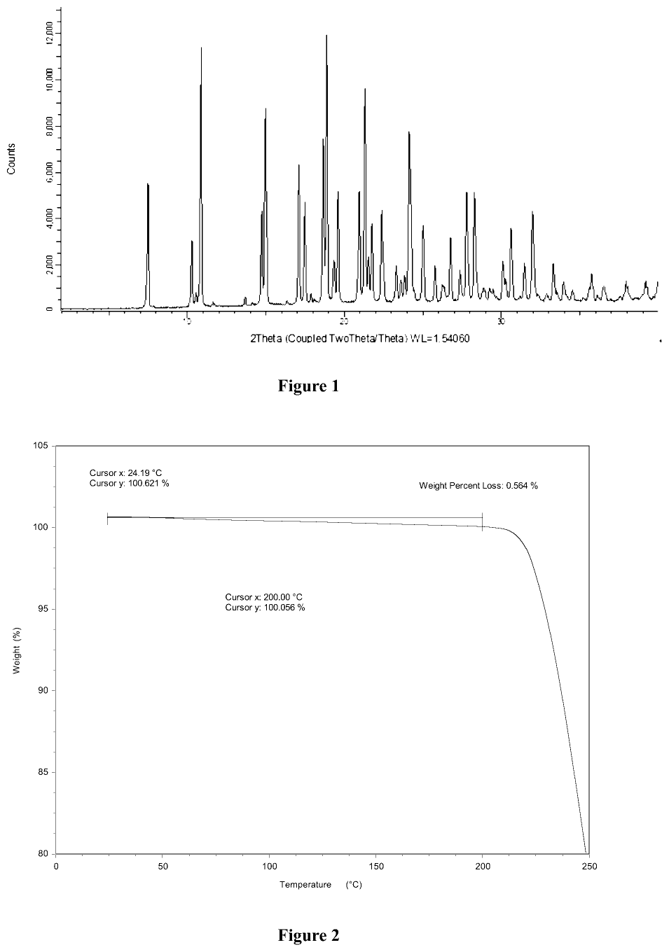

In some embodiments, the XRPD pattern of Crystalline Form A of Compound 1 is substantially as shown in FIG. 1. In some embodiments, the XRPD pattern of Crystalline Form A of Compound 1 is as shown in FIG. 1.

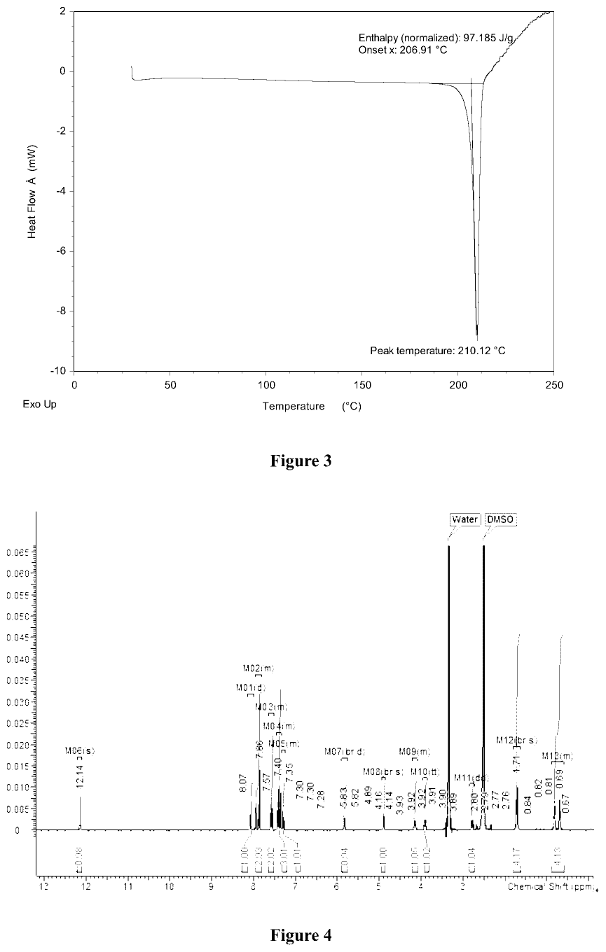

In some embodiments, the DSC curve of Crystalline Form A of Compound 1 comprises an endotherm at about 206.9° C. In some embodiments, the DSC curve of Crystalline Form A of Compound 1 is substantially as shown in FIG. 3. In some embodiments, the DSC curve of Crystalline Form A of Compound 1 is as shown in FIG. 3.

### III. Crystalline Form B of Compound 1

In another aspect, the present disclosure provides a Crystalline Form B of Compound 1. In some embodiments, the present disclosure provides a substantially pure Crystalline Form B of Compound 1. In some embodiments, Compound 1 is in a solvated crystalline form, for example, the Crystalline Form B of Compound 1 is a methanol channel solvate.

In some embodiments, the XRPD pattern of Crystalline Form B of Compound 1 comprises one or more (e.g., two, three) peaks selected from the group consisting of about 6.244°, about 18.679°, and about 12.445° 2θ. For example, the XRPD pattern of Crystalline Form B of Compound 1 comprises a peak at about 6.244° 2θ. For another example, the XRPD pattern of Crystalline Form B of Compound 1 comprises a peak at about 18.679° 2θ. For yet another example, the XRPD pattern of Crystalline Form B of Compound 1 comprises a peak at about 12.445° 2θ. For yet another example, the XRPD pattern of Crystalline Form B of Compound 1 comprises two peaks at about 6.244° and about 18.679° 2θ. For yet another example, the XRPD pattern of Crystalline Form B of Compound 1 comprises two peaks at about 6.244° and about 12.445° 2θ. For yet another example, the XRPD pattern of Crystalline Form B of Compound 1 comprises two peaks at about 18.679° and about 12.445° 2θ. In some embodiments, the XRPD pattern of Crystalline Form B of Compound 1 comprises all the peaks selected from the group consisting of about 6.244°, about 18.679°, and about 12.445° 2θ.

In some embodiments, the XRPD pattern of Crystalline Form B of Compound 1 further comprises one or more (e.g., 2, 3, 4, 5, 6, 7) peaks selected from the group consisting of about 17.239°, about 12.832°, about 23.559°, about 15.252°, about 22.410°, about 9.344°, and about 25.722° 2θ. For example, the XRPD pattern of Crystalline Form B of Compound 1 further comprises a peak at about 17.239°, about 12.832°, about 23.559°, about 15.252°, about 22.410°, about 9.344°, or about 25.722° 2θ. In some embodiments, the XRPD pattern of Crystalline Form B of Compound 1 further comprises all the peaks selected from the group consisting of about 17.239°, about 12.832°, about 23.559°, about 15.252°, about 22.410°, about 9.344°, and about 25.722° 2θ.

In some embodiments, the XRPD pattern of Crystalline Form B of Compound 1 further comprises one or more (e.g., 2, 3, 4, 5, 6, 7, 8, 9, 10, 11, 12, 13, 14, 15) peaks selected from the group consisting of about 20.354°, about 16.366°, about 24.731°, about 20.581°, about 28.664°, about 27.044°, about 35.758°, about 17.905°, about 16.087°, about 36.194°, about 34.867°, about 24.984°, about 10.802°, about 29.902°, and about 32.584° 2θ. For example, the XRPD pattern of Crystalline Form B of Compound 1 further comprises a peak at about 20.354°, about 16.366°, about 24.731°, about 20.5810, about 28.664°, about 27.044°, about 35.758°, about 17.905°, about 16.087°, about 36.194°, about 34.867°, about 24.984°, about 10.802°, about 29.902°, or about 32.584° 2θ. In some embodiments, the XRPD pattern of Crystalline Form B of Compound 1 further comprises all the peaks selected from the group consisting of about 20.354°, about 16.366°, about 24.731°, about 20.581°, about 28.664°, about 27.044°, about 35.758°, about 17.905°, about 16.087°, about 36.194°, about 34.867°, about 24.984°, about 10.802°, about 29.902°, and about 32.584° 2θ.

In some embodiments, the XRPD pattern of Crystalline Form B of Compound 1 comprises all the peaks selected from the group consisting of:

| Angle (°2θ) | Angle (°2θ) | Angle (°2θ) | Angle (°2θ) |
|---|---|---|---|
| 6.244 | 9.344 | 35.758 | 32.584 |
| 18.679 | 25.722 | 17.905 | 21.279 |
| 12.445 | 20.354 | 16.087 | 26.266 |
| 17.239 | 16.366 | 36.194 | 37.816 |
| 12.832 | 24.731 | 34.867 | 33.789 |
| 23.559 | 20.581 | 24.984 | 19.275 |
| 15.252 | 28.664 | 10.802 | 19.946 |
| 22.410 | 27.044 | 29.902 | 33.118 |

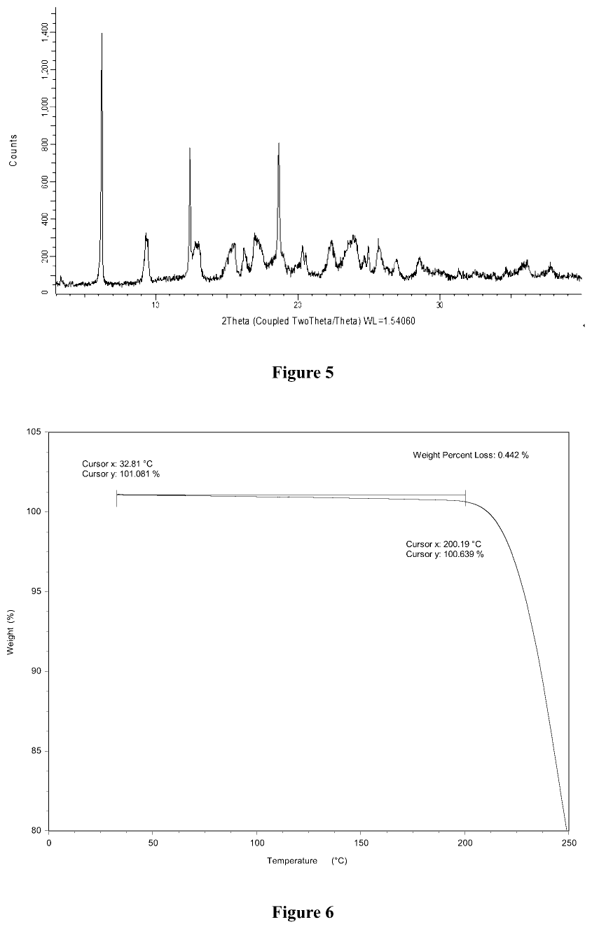

In some embodiments, the XRPD pattern of Crystalline Form B of Compound 1 is substantially as shown in FIG. 5. In some embodiments, the XRPD pattern of Crystalline Form B of Compound 1 is as shown in FIG. 5.

In some embodiments, the DSC curve of Crystalline Form B of Compound 1 comprises an endotherm at about 178.3° C., about 179.7° C., about 196.6° C., about 198.7° C., and/or about 203.7° C. In some embodiments, the DSC curve of Crystalline Form B of Compound 1 is substantially as shown in FIG. 7. In some embodiments, the DSC curve of Crystalline Form B of Compound 1 is as shown in FIG. 7.

### IV. Crystalline Form C of Compound 1

In another aspect, the present disclosure provides a Crystalline Form C of Compound 1. In some embodiments, the present disclosure provides a substantially pure Crystalline Form C of Compound 1. In some embodiments, Compound 1 is in a solvated crystalline form, for example, the Crystalline Form C of Compound 1 is a hydrate.

In some embodiments, the XRPD pattern of Crystalline Form C of Compound 1 comprises one or more (e.g., two, three) peaks selected from the group consisting of about 6.656°, about 19.994°, and about 26.732° 2θ. For example, the XRPD pattern of Crystalline Form C of Compound 1 comprises a peak at about 6.656° 2θ. For another example, the XRPD pattern of Crystalline Form C of Compound 1 comprises a peak at about 19.994° 2θ. For yet another example, the XRPD pattern of Crystalline Form C of Compound 1 comprises a peak at about 26.732° 2θ. For yet another example, the XRPD pattern of Crystalline Form C of Compound 1 comprises two peaks at about 6.6560 and about 19.994° 2θ. For yet another example, the XRPD pattern of Crystalline Form C of Compound 1 comprises two peaks at about 6.656° and about 26.732° 2θ. For yet another example, the XRPD pattern of Crystalline Form C of Compound 1 comprises two peaks at about 19.994° and about 26.732° 2θ. In some embodiments, the XRPD pattern of Crystalline Form C of Compound 1 comprises all the peaks selected from the group consisting of about 6.656°, about 19.994°, and about 26.732° 2θ.

In some embodiments, the XRPD pattern of Crystalline Form C of Compound 1 further comprises one or more (e.g., 2, 3, 4, 5, 6, 7) peaks selected from the group consisting of about 18.350°, about 20.753°, about 22.176°, about 15.941°, about 20.450°, about 13.320°, and about 17.687° 2θ. For example, the XRPD pattern of Crystalline Form C of Compound 1 further comprises a peak at about 18.350°, about 20.753°, about 22.176°, about 15.941°, about 20.450°, about 13.320°, or about 17.687° 2θ. In some embodiments, the XRPD pattern of Crystalline Form C of Compound 1 further comprises all the peaks selected from the group consisting of about 18.350°, about 20.753°, about 22.176°, about 15.941°, about 20.450°, about 13.320°, and about 17.687° 2θ.

In some embodiments, the XRPD pattern of Crystalline Form C of Compound 1 comprises all the peaks selected from the group consisting of:

| Angle (°2θ) | Angle (°2θ) | Angle (°2θ) |
|---|---|---|
| 6.656 | 20.450 | 12.470 |
| 19.994 | 13.320 | 3.724 |
| 26.732 | 17.687 | 25.036 |
| 18.350 | 19.205 | 30.741 |
| 20.753 | 23.302 | 4.454 |
| 22.176 | 9.183 | |
| 15.941 | 8.700 | |

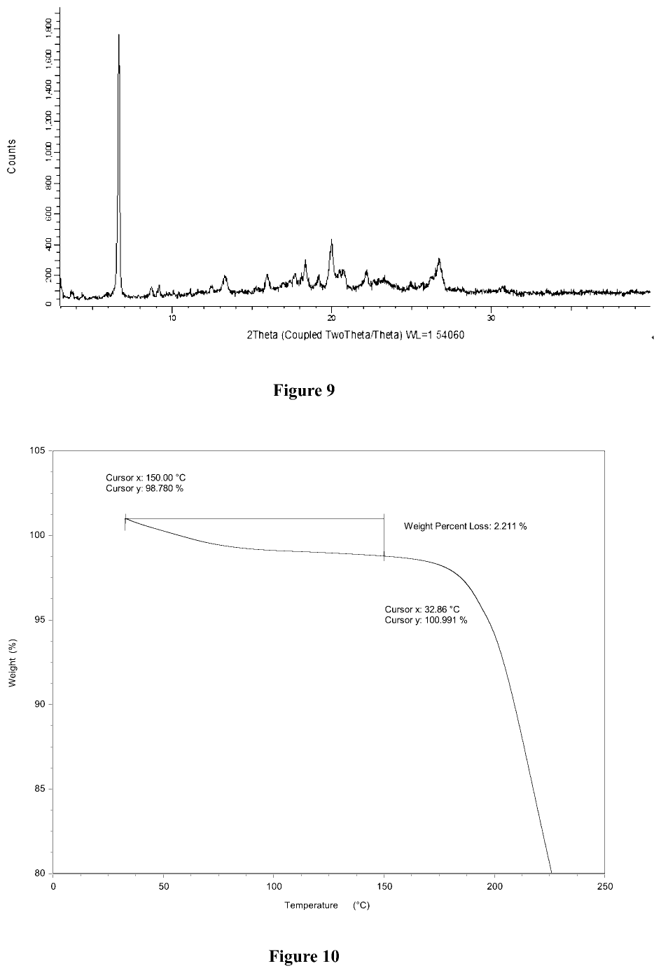

In some embodiments, the XRPD pattern of Crystalline Form C of Compound 1 is substantially as shown in FIG. 9. In some embodiments, the XRPD pattern of Crystalline Form C of Compound 1 is as shown in FIG. 9.

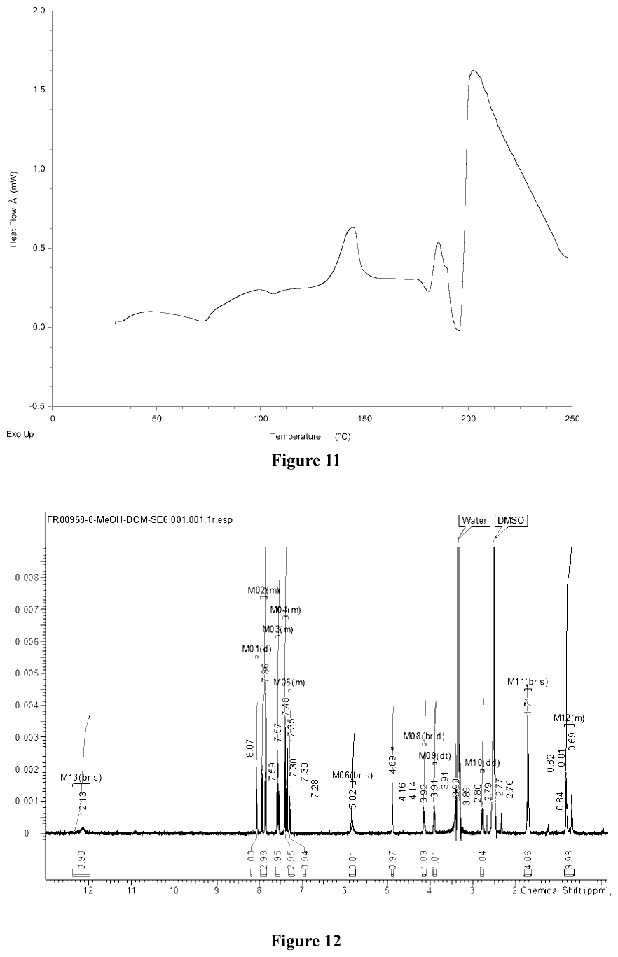

In some embodiments, the DSC curve of Crystalline Form C of Compound 1 is substantially as shown in FIG. 11. In some embodiments, the DSC curve of Crystalline Form C of Compound 1 is as shown in FIG. 11.

### V. Crystalline Form D of Compound 1

In another aspect, the present disclosure provides a Crystalline Form D of Compound 1. In some embodiments, the present disclosure provides a substantially pure Crystalline Form D of Compound 1. In some embodiments, Compound 1 is in an unsolvated crystalline form, for example, the Crystalline Form D of Compound 1 is an anhydrate.

In some embodiments, the XRPD pattern of Crystalline Form D of Compound 1 comprises one or more (e.g., two, three) peaks selected from the group consisting of about 16.956°, about 5.883°, and about 22.181° 2θ. For example, the XRPD pattern of Crystalline Form D of Compound 1 comprises a peak at about 16.956° 2θ. For another example, the XRPD pattern of Crystalline Form D of Compound 1 comprises a peak at about 5.883° 2θ. For yet another example, the XRPD pattern of Crystalline Form D of Compound 1 comprises a peak at about 22.181° 2θ. For yet another example, the XRPD pattern of Crystalline Form D of Compound 1 comprises two peaks at about 16.956° and about 5.883° 2θ. For yet another example, the XRPD pattern of Crystalline Form D of Compound 1 comprises two peaks at about 16.956° and about 22.181° 2θ. For yet another example, the XRPD pattern of Crystalline Form D of Compound 1 comprises two peaks at about 5.883° and about 22.181° 2θ. In some embodiments, the XRPD pattern of Crystalline Form D of Compound 1 comprises all the peaks selected from the group consisting of about 16.956°, about 5.883°, and about 22.181° 2θ.

In some embodiments, the XRPD pattern of Crystalline Form D of Compound 1 further comprises one or more (e.g., 2, 3, 4, 5, 6) peaks selected from the group consisting of about 17.663°, about 13.291°, about 22.909°, about 20.718°, about 26.900°, and about 18.113° 2θ. For example, the XRPD pattern of Crystalline Form D of Compound 1 further comprises a peak at about 17.663°, about 13.291°, about 22.909°, about 20.718°, about 26.900°, or about 18.113° 2θ. In some embodiments, the XRPD pattern of Crystalline Form D of Compound 1 further comprises all the peaks selected from the group consisting of about 17.663°, about 13.291°, about 22.909°, about 20.718°, about 26.900°, or about 18.113° 2θ.

In some embodiments, the XRPD pattern of Crystalline Form D of Compound 1 further comprises one or more (e.g., 2, 3, 4, 5, 6, 7, 8, 9, 10, 11, 12, 13, 14, 15) peaks selected from the group consisting of about 20.205°, about 11.727°, about 21.756°, about 22.617°, about 20.956°, about 29.635°, about 24.990°, about 12.566°, about 10.080°, about 24.078°, about 9.7610, about 19.581°, about 27.280°, about 25.637°, and about 12.754° 2θ. For example, the XRPD pattern of Crystalline Form D of Compound 1 further comprises a peak at about 20.205°, about 11.727°, about 21.756°, about 22.617°, about 20.956°, about 29.635°, about 24.990°, about 12.566°, about 10.080°, about 24.078°, about 9.761°, about 19.581°, about 27.280°, about 25.637°, or about 12.754° 2θ. In some embodiments, the XRPD pattern of Crystalline Form D of Compound 1 further comprises all the peaks selected from the group consisting of about 20.205°, about 11.727°, about 21.756°, about 22.617°, about 20.956°, about 29.635°, about 24.990°, about 12.566°, about 10.080°, about 24.078°, about 9.761°, about 19.581°, about 27.280°, about 25.637°, and about 12.754° 2θ.

In some embodiments, the XRPD pattern of Crystalline Form D of Compound 1 comprises all the peaks selected from the group consisting of:

| Angle (°2θ) | Angle (°2θ) | Angle (°2θ) | Angle (°2θ) |
|---|---|---|---|
| 16.956 | 21.756 | 25.637 | 39.200 |
| 5.883 | 22.617 | 12.754 | 33.344 |
| 22.181 | 20.956 | 14.335 | 18.891 |
| 17.663 | 29.635 | 35.599 | 34.841 |
| 13.291 | 24.990 | 10.411 | 10.830 |
| 22.909 | 12.566 | 30.457 | 28.859 |
| 20.718 | 10.080 | 11.067 | 14.954 |
| 26.900 | 24.078 | 19.175 | 28.254 |
| 18.113 | 9.761 | 34.373 | 31.083 |
| 20.205 | 19.581 | 36.486 | 7.456 |
| 11.727 | 27.280 | 37.185 | |

In some embodiments, the XRPD pattern of Crystalline Form D of Compound 1 is substantially as shown in FIG. 13. In some embodiments, the XRPD pattern of Crystalline Form D of Compound 1 is as shown in FIG. 13.

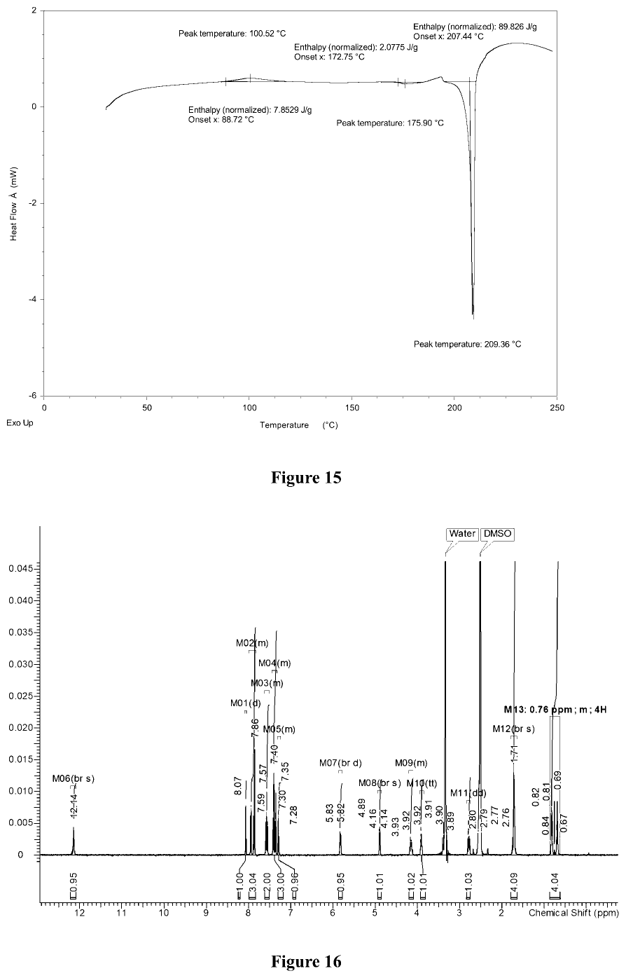

In some embodiments, the DSC curve of Crystalline Form D of Compound 1 comprises an endotherm at about 207.4° C. In some embodiments, the DSC curve of Crystalline Form D of Compound 1 is substantially as shown in FIG. 15. In some embodiments, the DSC curve of Crystalline Form D of Compound 1 is as shown in FIG. 15.

### VI. Crystalline Form E of Compound 1

In another aspect, the present disclosure provides a Crystalline Form E of Compound 1. In some embodiments, the present disclosure provides a substantially pure Crystalline Form E of Compound 1. In some embodiments, Compound 1 is in an unsolvated crystalline form, for example, the Crystalline Form E of Compound 1 is an anhydrate.

In some embodiments, the XRPD pattern of Crystalline Form E of Compound 1 comprises one or more (e.g., two, three) peaks selected from the group consisting of about 18.089°, about 26.315°, and about 19.775° 2θ. For example, the XRPD pattern of Crystalline Form E of Compound 1 comprises a peak at about 18.089° 2θ. For another example, the XRPD pattern of Crystalline Form E of Compound 1 comprises a peak at about 26.315° 2θ. For yet another example, the XRPD pattern of Crystalline Form E of Compound 1 comprises a peak at about 19.775° 2θ. For yet another example, the XRPD pattern of Crystalline Form E of Compound 1 comprises two peaks at about 18.089° and about 26.315° 2θ. For yet another example, the XRPD pattern of Crystalline Form E of Compound 1 comprises two peaks at about 18.089° and about 19.775° 2θ. For yet another example, the XRPD pattern of Crystalline Form E of Compound 1 comprises two peaks at about 26.315° and about 19.775° 2θ. In some embodiments, the XRPD pattern of Crystalline Form E of Compound 1 comprises all the peaks selected from the group consisting of about 18.089°, about 26.315°, and about 19.775° 2θ.

In some embodiments, the XRPD pattern of Crystalline Form E of Compound 1 further comprises one or more (e.g., 2, 3, 4, 5) peaks selected from the group consisting of about 24.177°, about 20.803°, about 16.018°, about 21.429°, and about 14.608° 2θ. the XRPD pattern of Crystalline Form E of Compound 1 further comprises a peak at about 24.177°, about 20.803°, about 16.018°, about 21.429°, or about 14.608° 2θ. In some embodiments, the XRPD pattern of Crystalline Form E of Compound 1 further comprises all the peaks selected from the group consisting of about 24.177°, about 20.803°, about 16.018°, about 21.429°, and about 14.608° 2θ.

In some embodiments, the XRPD pattern of Crystalline Form E of Compound 1 further comprises one or more (e.g., 2, 3, 4, 5) peaks selected from the group consisting of about 21.314°, about 7.794°, about 20.007°, about 8.744°, and about 15.573° 2θ. For example, the XRPD pattern of Crystalline Form E of Compound 1 further comprises a peak at about 21.314°, about 7.794°, about 20.007°, about 8.744°, or about 15.573° 2θ. In some embodiments, the XRPD pattern of Crystalline Form E of Compound 1 further comprises all the peaks selected from the group consisting of about 21.314°, about 7.794°, about 20.007°, about 8.744°, and about 15.573° 2θ.

In some embodiments, the XRPD pattern of Crystalline Form E of Compound 1 comprises all the peaks selected from the group consisting of:

| Angle (°2θ) | Angle (°2θ) | Angle (°2θ) | Angle (°2θ) |
|---|---|---|---|
| 18.089 | 18.636 | 27.764 | 29.879 |
| 26.315 | 28.686 | 17.267 | 34.904 |
| 19.775 | 16.43 | 14.962 | 32.851 |
| 24.177 | 25.182 | 18.864 | 35.467 |
| 20.803 | 20.584 | 12.049 | 31.997 |
| 16.018 | 10.856 | 17.085 | 12.878 |
| 21.429 | 22.515 | 7.486 | 11.239 |
| 14.608 | 19.611 | 17.477 | 30.606 |
| 21.314 | 14.233 | 13.613 | 3.691 |
| 7.794 | 22.83 | 36.59 | 34.151 |
| 20.007 | 23.557 | 28.293 | 33.397 |
| 8.744 | 27.166 | 21.743 | |
| 15.573 | 24.647 | 31.524 | |

In some embodiments, the XRPD pattern of Crystalline Form E of Compound 1 is substantially as shown in FIG. 17. In some embodiments, the XRPD pattern of Crystalline Form E of Compound 1 is as shown in FIG. 17.

### VII. Crystalline Form A of L-malate Salt of Compound 1

In another aspect, the present disclosure provides a Crystalline Form A of malate salt of Compound 1. In some embodiments, the present disclosure provides a Crystalline Form A of L-malate Salt of Compound 1.

In some embodiments, the XRPD pattern of Crystalline Form A of L-malate Salt of Compound 1 comprises one or more (e.g., two, three) peaks selected from the group consisting of about 19.349°, about 22.304°, and about 20.078° 2θ. For example, the XRPD pattern of Crystalline Form A of L-malate Salt of Compound 1 comprises a peak at about 19.349° 2θ. For another example, the XRPD pattern of Crystalline Form A of L-malate Salt of Compound 1 comprises a peak at about 22.304° 2θ. For yet another example, the XRPD pattern of Crystalline Form A of L-malate Salt of Compound 1 comprises a peak at about 20.078° 2θ. For yet another example, the XRPD pattern of Crystalline Form A of L-malate Salt of Compound 1 comprises two peaks at about 19.349° and about 22.304° 2θ. For yet another example, the XRPD pattern of Crystalline Form A of L-malate Salt of Compound 1 comprises two peaks at about 19.349° and about 20.078° 2θ. For yet another example, the XRPD pattern of Crystalline Form A of L-malate Salt of Compound 1 comprises two peaks at about 22.304° and about 20.078° 2θ. In some embodiments, the XRPD pattern of Crystalline Form A of L-malate Salt of Compound 1 comprises all the peaks selected from the group consisting of about 19.349°, about 22.304°, and about 20.078° 2θ.

In some embodiments, the XRPD pattern of Crystalline Form A of L-malate Salt of Compound 1 further comprises one or more (e.g., 2, 3, 4, 5, 6, 7, 8, 9, 10, 11, 12) peaks selected from the group consisting of about 24.489°, about 22.982°, about 18.186°, about 19.083°, about 6.369°, about 12.702°, about 24.937°, about 28.749°, about 24.407°, about 23.772°, about 23.597°, and about 17.581° 2θ. For example, the XRPD pattern of Crystalline Form A of L-malate Salt of Compound 1 further comprises a peak at about 24.489°, about 22.982°, about 18.186°, about 19.083°, about 6.369°, about 12.702°, about 24.937°, about 28.749°, about 24.407°, about 23.772°, about 23.597°, or about 17.581° 2θ. In some embodiments, the XRPD pattern of Crystalline Form A of L-malate Salt of Compound 1 further comprises all the peaks selected from the group consisting of about 24.489°, about 22.982°, about 18.186°, about 19.083°, about 6.369°, about 12.702°, about 24.937°, about 28.749°, about 24.407°, about 23.772°, about 23.597°, and about 17.581° 2θ.

In some embodiments, the XRPD pattern of Crystalline Form A of L-malate Salt of Compound 1 further comprises one or more (e.g., 2, 3, 4, 5, 6, 7, 8, 9, 10, 11, 12, 13, 14, 15, 16, 17) peaks selected from the group consisting of about 16.263°, about 31.742°, about 18.334°, about 25.587°, about 3.208°, about 8.765°, about 9.529°, about 20.434°, about 30.739°, about 18.878°, about 26.992°, about 21.941°, about 20.948°, about 36.810°, about 34.693°, about 32.704°, and about 21.715° 2θ. For example, the XRPD pattern of Crystalline Form A of L-malate Salt of Compound 1 further comprises a peak at about 16.263°, about 31.742°, about 18.334°, about 25.587°, about 3.208°, about 8.765°, about 9.529°, about 20.434°, about 30.739°, about 18.878°, about 26.992°, about 21.941°, about 20.948°, about 36.810°, about 34.693°, about 32.704°, or about 21.715° 2θ. For another example, the XRPD pattern of Crystalline Form A of L-malate Salt of Compound 1 further comprises all the peaks selected from the group consisting of about 16.263°, about 31.742°, about 18.334°, about 25.587°, about 3.208°, about 8.765°, about 9.529°, about 20.434°, about 30.739°, about 18.878°, about 26.992°, about 21.941°, about 20.948°, about 36.810°, about 34.693°, about 32.704°, and about 21.715° 2θ.

In some embodiments, the XRPD pattern of Crystalline Form A of L-malate Salt of Compound 1 comprises all the peaks selected from the group consisting of: about 19.349°, about 22.304°, about 20.078°, about 24.489°, about 22.982°, about 18.186°, about 19.083°, about 6.369°, about 12.702°, about 24.937°, about 28.749°, about 24.407°, about 23.772°, about 23.597°, about 17.581°, about 16.263°, about 31.742°, about 18.334°, about 25.587°, about 3.208°, about 8.765°, about 9.529°, about 20.434°, about 30.739°, about 18.878°, about 26.992°, about 21.941°, about 20.948°, about 36.810°, about 34.693°, about 32.704°, about 21.715°, about 36.432°, about 31.492°, about 27.976°, about 37.328°, about 17.975°, about 38.681°, about 15.886°, about 13.678°, about 32.223°, about 26.405°, about 27.514°, about 31.082°, about 14.561°, about 39.38°, about 35.442°, about 26.035°, about 26.739°, about 35.592°, about 11.409°, about 10.2°, about 8.962°, about 34.334°, about 28.307°, about 35.984°, about 29.228°, about 39.705°, about 33.715°, and about 30.135° 2θ.

In some embodiments, the XRPD pattern of Crystalline Form A of L-malate Salt of Compound 1 is substantially as shown in FIG. 18. In some embodiments, the XRPD pattern of Crystalline Form A of L-malate Salt of Compound 1 is as shown in FIG. 18.

In some embodiments, the DSC curve of Crystalline Form A of L-malate Salt of Compound 1 comprises an endotherm at about 160.3° C. In some embodiments, the DSC curve of Crystalline Form A of L-malate Salt of Compound 1 is substantially as shown in FIG. 20. In some embodiments, the DSC curve of Crystalline Form A of L-malate Salt of Compound 1 is as shown in FIG. 20.

### VIII. Crystalline Form A of L-tartrate Salt of Compound 1

In another aspect, the present disclosure provides a tartrate salt of Crystalline Form A of Compound 1. In some embodiments, the present disclosure provides a Crystalline Form A of L-tartrate Salt of Compound 1.

In some embodiments, the XRPD pattern of Crystalline Form A of L-tartrate Salt of Compound 1 comprises one or more (e.g., two, three) peaks selected from the group consisting of about 18.564°, about 21.685°, and about 18.774° 2θ. For example, the XRPD pattern of Crystalline Form A of L-tartrate Salt of Compound 1 comprises a peak at about 18.564° 2θ. For another example, the XRPD pattern of Crystalline Form A of L-tartrate Salt of Compound 1 comprises a peak at about 21.685° 2θ. For yet another example, the XRPD pattern of Crystalline Form A of L-tartrate Salt of Compound 1 comprises a peak at about 18.774° 2θ. For yet another example, the XRPD pattern of Crystalline Form A of L-tartrate Salt of Compound 1 comprises two peaks at about 18.564° and about 21.685° 2θ. For yet another example, the XRPD pattern of Crystalline Form A of L-tartrate Salt of Compound 1 comprises two peaks at about 21.685° and about 18.774° 2θ. For yet another example, the XRPD pattern of Crystalline Form A of L-tartrate Salt of Compound 1 comprises two peaks at about 18.564° and about 18.774° 2θ. In some embodiments, the XRPD pattern of Crystalline Form A of L-tartrate Salt of Compound 1 comprises all the peaks selected from the group consisting of about 18.564°, about 21.685° and about 18.774° 2θ.

In some embodiments, the XRPD pattern of Crystalline Form A of L-tartrate Salt of Compound 1 further comprises one or more (e.g., 2, 3, 4, 5, 6) peaks selected from the group consisting of about 24.834°, about 22.437°, about 37.586°, about 24.428°, about 20.965°, and about 24.193° 2θ. For example, the XRPD pattern of Crystalline Form A of L-tartrate Salt of Compound 1 further comprises a peak at about 24.834°, about 22.437°, about 37.586°, about 24.428°, about 20.965°, or about 24.193° 2θ. In some embodiments, the XRPD pattern of Crystalline Form A of L-tartrate Salt of Compound 1 further comprises all the peaks selected from the group consisting of about 24.834°, about 22.437°, about 37.586°, about 24.428°, about 20.965°, and about 24.193° 2θ.

In some embodiments, the XRPD pattern of Crystalline Form A of L-tartrate Salt of Compound 1 further comprises one or more (e.g., 2, 3, 4, 5, 6, 7, 8, 9, 10, 11, 12, 13, 14, 15, 16, 17, 18) peaks selected from the group consisting of about 17.789°, about 3.135°, about 19.49°, about 25.427°, about 31.744°, about 16.413°, about 9.279°, about 26.623°, about 12.36°, about 27.826°, about 14.438°, about 12.221°, about 24.045°, about 31.932°, about 19.234°, about 28.295°, about 20.641°, and about 19.08° 2θ. For example, the XRPD pattern of Crystalline Form A of L-tartrate Salt of Compound 1 further comprises a peak at about 17.789°, about 3.135°, about 19.49°, about 25.427°, about 31.744°, about 16.413°, about 9.279°, about 26.623°, about 12.36°, about 27.826°, about 14.438°, about 12.221°, about 24.045°, about 31.932°, about 19.234°, about 28.295°, about 20.641°, or about 19.08° 2θ. In some embodiments, the XRPD pattern of Crystalline Form A of L-tartrate Salt of Compound 1 further comprises all the peaks selected from the group consisting of about 17.789°, about 3.135°, about 19.49°, about 25.427°, about 31.744°, about 16.413°, about 9.279°, about 26.623°, about 12.36°, about 27.826°, about 14.438°, about 12.221°, about 24.045°, about 31.932°, about 19.234°, about 28.295°, about 20.641°, and about 19.08° 2θ.

In some embodiments, the XRPD pattern of Crystalline Form A of L-tartrate Salt of Compound 1 further comprises all the peaks selected from the group consisting of about 18.564°, about 21.685°, about 18.774°, about 24.834°, about 22.437°, about 37.586°, about 24.428°, about 20.965°, about 24.193°, about 17.789°, about 3.135°, about 19.49°, about 25.427°, about 31.744°, about 16.413°, about 9.279°, about 26.623°, about 12.36°, about 27.826°, about 14.438°, about 12.221°, about 24.045°, about 31.932°, about 19.234°, about 28.295°, about 20.641°, about 19.08°, about 10.165°, about 33.761°, about 29.071°, about 25.916°, about 16.943°, about 32.967°, about 23.691°, about 22.788°, about 34.232°, about 35.17°, about 34.812°, about 27.271°, about 32.24°, about 39.082°, about 30.774°, about 15.453°, about 30.534°, about 31.183°, about 37.181°, about 35.805°, about 28.554°, about 29.645°, about 39.789°, about 38.258°, about 10.432°, about 13.968°, and about 36.619° 2θ.

In some embodiments, the XRPD pattern of Crystalline Form A of L-tartrate Salt of Compound 1 is substantially as shown in FIG. 22. In some embodiments, the XRPD pattern of Crystalline Form A of L-tartrate Salt of Compound 1 is as shown in FIG. 22.

In some embodiments, the DSC curve of Crystalline Form A of L-tartrate Salt of Compound 1 comprises an endotherm at about 189.9° C. In some embodiments, the DSC curve of Crystalline Form A of L-tartrate Salt of Compound 1 is substantially as shown in FIG. 24. In some embodiments, the DSC curve of Crystalline Form A of L-tartrate Salt of Compound 1 is as shown in FIG. 24.

### IX. Pharmaceutical Compositions

In another aspect, the present disclosure provides a pharmaceutical composition comprising Compound 1 or a pharmaceutical salt thereof, and a pharmaceutically acceptable excipient, wherein Compound 1 comprises a crystalline form selected from the group consisting of the Crystalline Form A, Crystalline Form B, Crystalline Form C, Crystalline Form D, Crystalline Form E of Compound 1, the Crystalline Form A of L-malate Salt of Compound 1, and the Crystalline Form A of L-tartrate Salt of Compound 1 described herein.

A pharmaceutical composition, as used herein, refers to a mixture of one or more crystalline forms of Compound 1, or a pharmaceutical salt thereof described herein with other chemical components, such as carriers, stabilizers, diluents, dispersing agents, suspending agents, thickening agents, and/or excipients. In some embodiments, a pharmaceutical composition facilitates administration of one or more crystalline forms of Compound 1, or a pharmaceutical salt thereof described herein to an organism. In some embodiments, practicing the methods of treatment or use provided herein, includes administering or using a pharmaceutical composition comprising a therapeutically effective amount of a crystalline form of Compound 1, or a pharmaceutical salt thereof described herein. In specific embodiments, the methods of treatment provided for herein include administering such a pharmaceutical composition to a mammal having a disease or condition to be treated. In one embodiment, the mammal is a human. In some embodiments, the therapeutically effective amount varies widely depending on the severity of the disease, the age and relative health of the subject, the potency of the compound used and other factors. In various embodiments, the crystalline form of Compound 1, or a pharmaceutical salt thereof described herein are used singly or in combination with one or more additional therapeutic agents as components of mixtures.

In some embodiments, pharmaceutical compositions are formulated in any manner, including using one or more physiologically acceptable carriers comprising excipients and/or auxiliaries which facilitate processing of one or more of the crystalline forms of Compound 1, or a pharmaceutical salt thereof described herein into pharmaceutical preparations. In some embodiments, proper formulation is dependent upon the route of administration chosen. In various embodiments, any techniques, carriers, and excipients are used as suitable.

In some embodiments, the composition is formulated in a formulation for local or systemic delivery. Examples of such formulations are formulations for oral administration, injection, topical administration, pulmonary administration, or implant.

In some embodiments, the pharmaceutical compositions provided herein are formulated for intravenous injections. In certain aspects, the intravenous injection formulations provided herein are formulated as aqueous solutions, and, in some embodiments, in physiologically compatible buffers such as Hank's solution, Ringer's solution, or physiological saline buffer.

In certain embodiments, the pharmaceutical compositions provided herein are formulated for transmucosal administration. In some embodiments, transmucosal formulations include penetrants appropriate to the barrier to be permeated.

In certain embodiments, the pharmaceutical compositions provided herein are formulated for other parenteral injections, appropriate formulations include aqueous or nonaqueous solutions, and in one embodiment, with physiologically compatible buffers or excipients.

In some embodiments, parenteral injections involve bolus injection or continuous infusion. In some embodiments, formulations for injection are presented in unit dosage form, e.g., in ampoules or in multi-dose containers, with an added preservative. In some embodiments, the pharmaceutical composition described herein is in a form suitable for parenteral injection as a sterile suspensions, solutions or emulsions in oily or aqueous vehicles, and optionally contains formulatory agents such as suspending, stabilizing and/or dispersing agents. Pharmaceutical formulations for parenteral administration include aqueous solutions of the active ingredients in water-soluble form. In some embodiments, suspensions of the active ingredients are prepared as appropriate oily injection suspensions. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. Optionally, the suspensions also contain suitable stabilizers or agents which increase the solubility of the compounds to allow for the preparation of highly concentrated solutions. In alternative embodiments, the active ingredient is in powder form for constitution with a suitable vehicle, e.g., sterile pyrogen-free water, before use.

In some embodiments, the pharmaceutical compositions provided herein are formulated for oral administration. In certain aspects, the oral formulations provided herein comprise one or more crystalline forms of Compound 1 or a pharmaceutical salt thereof described herein that are formulated with pharmaceutically acceptable carriers or excipients. Such carriers enable the crystalline forms of Compound 1 or a pharmaceutical salt thereof described herein to be formulated as tablets, powders, pills, dragees, capsules, liquids, gels, syrups, elixirs, slurries, suspensions and the like, for oral ingestion by a patient to be treated.

In some embodiments, pharmaceutical compositions for oral use are obtained by mixing one or more solid excipient with one or more of the crystalline forms of Compound 1 or a pharmaceutical salt thereof described herein, optionally grinding the resulting mixture, and processing the mixture of granules, after adding suitable auxiliaries, if desired, to obtain tablets or dragee cores. Suitable excipients include, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are optionally added, such as the cross-linked croscarmellose sodium, polyvinylpyrrolidone, agar, or alginic acid or a salt thereof such as sodium alginate.

In some embodiments, provided herein is a pharmaceutical composition formulated as dragee cores with suitable coatings. In some embodiments, concentrated sugar solutions are used in forming the suitable coating, and optionally contain gum arabic, talc, polyvinylpyrrolidone, carbopol gel, polyethylene glycol, and/or titanium dioxide, lacquer solutions, and suitable organic solvents or solvent mixtures. In some embodiments, dyestuffs and/or pigments are added to tablets, dragees and/or the coatings thereof for, e.g., identification or to characterize different combinations of active compound doses.

In some embodiments, pharmaceutical compositions which are used include orally include push-fit capsules made of gelatin, as well as soft, sealed capsules made of gelatin and a plasticizer, such as glycerol or sorbitol. In some embodiments, the push-fit capsules contain the active ingredients in admixture with filler such as lactose, binders such as starches, and/or lubricants such as talc or magnesium stearate and, optionally, stabilizers. In some embodiments, in soft capsules, the active ingredients are dissolved or suspended in suitable liquids, such as fatty oils, liquid paraffin, or liquid polyethylene glycols. In addition, stabilizers are optionally added. In some or any embodiments, the formulations for oral administration are in dosages suitable for such administration.

In some embodiments, the pharmaceutical compositions provided herein are formulated for buccal or sublingual administration. In some embodiments, buccal or sublingual compositions take the form of tablets, lozenges, or gels formulated in a conventional manner.

In some embodiments, the pharmaceutical compositions provided herein are formulated for transdermal administration. In some embodiments, administration of such compositions employs transdermal delivery devices and transdermal delivery patches. In some or any embodiments, the compositions are lipophilic emulsions or buffered, aqueous solutions, dissolved and/or dispersed in a polymer or an adhesive. Such patches include those constructed for continuous, pulsatile, or on demand delivery of pharmaceutical agents. In some embodiments, transdermal delivery of one or more crystalline forms of Compound 1 or a pharmaceutical salt thereof described herein is accomplished by use of iontophoretic patches and the like. In some or any embodiments, transdermal patches provide controlled delivery of one or more crystalline forms of Compound 1 or a pharmaceutical salt thereof described herein. In some embodiments, the rate of absorption is slowed by using rate-controlling membranes or by trapping the compound within a polymer matrix or gel. Conversely, absorption enhancers are optionally used to increase absorption. Absorption enhancer and carrier include absorbable pharmaceutically acceptable solvents that assist in passage of the compound through the skin. For example, transdermal devices are in the form of a bandage comprising a backing member, a reservoir containing one or more crystalline forms of Compound 1 or a pharmaceutical salt thereof described herein optionally with carriers, optionally a rate controlling barrier to deliver the compound to the skin of the host at a controlled and predetermined rate over a prolonged period of time, and means to secure the device to the skin.

In some or any embodiments, the pharmaceutical compositions provided herein are formulated for administration by inhalation. In some embodiments, in such pharmaceutical compositions formulated for inhalation, the one or more crystalline forms of Compound 1 or a pharmaceutical salt thereof described herein are in a form as an aerosol, a mist or a powder. In some embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In certain aspects of a pressurized aerosol, the dosage unit is determined by providing a valve to deliver a metered amount. In some or any embodiments, capsules and cartridges of, such as, by way of example only, gelatin for use in an inhaler or insufflator is formulated containing a powder mix of the one or more crystalline forms of Compound 1 or a pharmaceutical salt thereof described herein and a suitable powder base such as lactose or starch.

In some embodiments, the compounds described herein are formulated in rectal compositions such as enemas, rectal gels, rectal foams, rectal aerosols, suppositories, jelly suppositories, or retention enemas. In some embodiments, rectal compositions optionally contain conventional suppository bases such as cocoa butter or other glycerides, as well as synthetic polymers such as polyvinylpyrrolidone, PEG, and the like. In some or any suppository forms of the compositions, a low-melting wax such as, but not limited to, a mixture of fatty acid glycerides, optionally in combination with cocoa butter is first melted.

Methods for the preparation of compositions containing the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. In various embodiments, the compositions are in liquid solutions or suspensions, solid forms suitable for solution or suspension in a liquid prior to use, or as emulsions. These compositions optionally contain minor amounts of nontoxic, auxiliary substances, such as wetting or emulsifying agents, pH buffering agents, and so forth.

In some embodiments, a composition comprising a compound described herein takes the form of a liquid where the agents are present in solution, in suspension or both. In some embodiments, when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

Useful aqueous suspension optionally contains one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions optionally comprise an mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions optionally include solubilizing agents to aid in the solubility of a compound described herein. The term “solubilizing agent” generally includes agents that result in formation of a micellar solution or a true solution of the agent. Solubilizing agents include certain acceptable nonionic surfactants, for example polysorbate 80, and ophthalmically acceptable glycols, polyglycols, e.g., polyethylene glycol 400, and glycol ethers.

Useful compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Useful compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Certain useful compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Some useful compositions optionally include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Certain useful compositions optionally one or more antioxidants to enhance chemical stability where required. Suitable antioxidants include, by way of example only, ascorbic acid and sodium metabisulfite.

In some embodiments, aqueous suspension compositions are packaged in single-dose non-reclosable containers. In alternative embodiments, multiple-dose reclosable containers are used, in which case it is typical to include a preservative in the composition.

In various embodiments, any delivery system for hydrophobic pharmaceutical compounds is employed. Liposomes and emulsions are examples of delivery vehicles or carriers for hydrophobic drugs. In some or any embodiments, certain organic solvents such as N-methylpyrrolidone are employed. In some embodiments, the compounds are delivered using a sustained-release system, such as semipermeable matrices of solid hydrophobic polymers containing the therapeutic agent. Various sustained-release materials are utilized in the embodiments herein. In some or any embodiments, sustained-release capsules release the compounds for a few weeks up to over 100 days. In some embodiments, depending on the chemical nature and the biological stability of the therapeutic reagent, additional strategies for protein stabilization are employed.

In some or any embodiments, the formulations or compositions described herein benefit from and/or optionally comprise metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.10% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some or any embodiments, the pharmaceutical compositions described herein are in a unit dosage form suitable for single administration of precise dosages. In some instances, in unit dosage form, the formulation is divided into unit doses containing appropriate quantities of one or more compound. In some or any embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. In alternative embodiments, multiple-dose reclosable containers are used, in which case it is typical to include a preservative in the composition. By way of example only, formulations for parenteral injection are, in some embodiments, presented in unit dosage form, which include, but are not limited to ampoules, or in multi-dose containers, with an added preservative.

### X. Uses

In another aspect, the present disclosure also provides use of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein in the manufacture of a medicament for:
(a) preventing or treating a disease, disorder or condition that is associated with an abnormal activity/level of the enzyme GCS;
(b) preventing or treating a GCS-mediated disease, disorder or condition;
(c) inhibiting an activity/level of the enzyme GCS; or
(d) non-therapeutically inhibiting an activity/level of the enzyme GCS in vitro.

The phrase “GCS-mediated disease, disorder or condition” refers to a disease, disorder or condition that is associated with an abnormal increased or decreased activity/level of the enzyme GCS, which may be caused by the abnormal activation or inactivation of the enzyme GCS in a subject. In some embodiments, the GCS-mediated disease, disorder or condition is a disease, disorder or condition that is associated with an abnormal increased activity/level of the enzyme GCS.

In some embodiments, the disease, disorder or condition that is associated with an abnormal activity/level of the enzyme GCS, or the GCS-mediated disease, disorder or condition is selected from the group consisting of a glycolipid storage disease (e.g., Tay Sachs, Sandhoffs, GMl gangliosidosis and Fabry diseases); a disease associated with glycolipid accumulation (e.g., Gaucher disease); a disease that cause renal hypertrophy or hyperplasia such as diabetic nephropathy; a disease that cause hyperglycemia or hyperinsulemia; a cancer in which glycolipid synthesis is abnormal; an infectious disease caused by organisms which use cell surface glycolipids as receptors or in which synthesis of glucosylceramide is essential or important; a metabolic disorder such as atherosclerosis, polycystic kidney disease, renal hypertrophy, and diabetes mellitus; obesity; a cancer such as breast cancer, renal adenocarcinoma, brain cancer, neuroblastoma, lung cancer, intestinal cancer, pancreas and prostrate cancer; a neuronal disorder; neuronal injury; an inflammatory disease or disorder (e.g., rheumatoid arthritis, Crohn's disease, asthma and sepsis); pain (e.g., neuropathic pain, inflammatory pain, headache pain, somatic pain, visceral pain, referred pain); a cognitive disorder (e.g., agnosia; amnesia; aphasia; an apraxia; delirium; dementia including AIDS dementia complex, Binswanger's disease, dementia with Lewy Bodies, frontotemporal dementia, mild cognitive impairment, multi-infarct dementia, Pick's disease, semantic dementia, senile dementia, and vascular dementia; and learning disorders including Asperger's syndrome, attention deficit disorder, attention deficit hyperactivity disorder, autism, childhood disintegrative disorder, and Rett syndrome), a neurodegenerative disorder (such as Alzheimer's disease, corticobasal degeneration, Creutzfeldt-Jacob disease, frontotemporal lobar degeneration, Huntington disease, multiple sclerosis, normal pressure hydrocephalus, organic chronic brain syndrome, Parkinson's disease, Pick disease, progressive supranuclear palsy, and senile dementia (Alzheimer type), glomerular disease), and nonalcoholic fatty liver disease (NALD). In some embodiments, the disease, disorder or condition is Tay Sachs, Sandhoffs, GMl gangliosidosis, Fabry disease, Gaucher disease, or polycystic kidney disease.

In another aspect, the present disclosure also provides a method for preventing or treating a GCS-mediated disease, disorder or condition in a subject, comprising administering to the subject the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein.

In another aspect, the present disclosure also provides the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein for use in preventing or treating a GCS-mediated disease, disorder or condition.

In another aspect, the present disclosure also provides a method of inhibiting GCS activity, comprising administering to a subject in need thereof an effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein.

### XI. Methods of Dosing and Treatment of Regimens

In any of the aforementioned embodiments are further embodiments that include single administrations of the effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein, including further embodiments in which the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is administered to the subject (i) once; (ii) multiple times over the span of one day; (iii) continually; or (iv) continuously.

In any of the aforementioned embodiments are further embodiments in which administration is enteral, parenteral, or both, and wherein:
(a) the effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is systemically administered to the subject;
(b) the effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is orally administered to the subject;
(c) the effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is intravenously administered to the subject;
(d) the effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is administered to the subject by inhalation;
(e) the effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is administered to the subject by nasal administration;
(f) the effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is administered to the subject by injection;
(g) the effective amount of the Crystalline Form A, Crystalline Form B, Crystalline Form C, Crystalline Form D, Crystalline Form E of Compound 1, a pharmaceutically acceptable salt thereof, or a pharmaceutical composition described herein is administered topically (dermal) to the subject;
(h) the effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is administered to the subject by ophthalmic administration; and/or
(i) the effective amount of the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is administered rectally to the subject.

In some or any embodiments, the crystalline forms of Compound 1, a pharmaceutical salt thereof, or a pharmaceutical composition described herein are administered for prophylactic and/or therapeutic treatments. In some or any therapeutic applications, the crystalline forms of Compound 1, a pharmaceutical salt thereof, or a pharmaceutical composition described herein are administered to a patient already suffering from a disease or condition, in an amount sufficient to cure or at least partially arrest the symptoms of the disease or condition. In some embodiments, amounts effective for this use will depend on the severity and course of the disease or condition, previous therapy, the patient's health status, weight, and response to the drugs, and the judgment of the treating physician. In certain instances, it is considered appropriate for the caregiver to determine such therapeutically effective amounts by routine experimentation (including, but not limited to, a dose escalation clinical trial).

In some or any prophylactic applications, the crystalline forms of Compound 1, a pharmaceutical salt thereof, or a pharmaceutical composition described herein are administered to a patient susceptible to or otherwise at risk of a particular disease, disorder or condition. In some embodiments, the amount administered is defined to be a “prophylactically effective amount or dose.” In some or any embodiments of this use, the precise amounts of the crystalline forms of Compound 1, a pharmaceutical salt thereof, or a pharmaceutical composition described herein administered depend on the patient's state of health, weight, and the like. In some embodiments, it is considered appropriate for the caregiver to determine such prophylactically effective amounts by routine experimentation (e.g., a dose escalation clinical trial). In some or any embodiments, when used in a patient, effective amounts for this use will depend on the severity and course of the disease, disorder or condition, previous therapy, the patient's health status and response to the drugs, and the judgment of the treating physician.

In some or any embodiments are further embodiments that include multiple administrations of the effective amount of the compound, including further embodiments wherein:
(a) the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is administered in a single dose;
(b) the time between multiple administrations is every 6 hours; or
(c) the Crystalline Form A, Crystalline Form B, Crystalline Form C, Crystalline Form D, Crystalline Form E of Compound 1, a pharmaceutically acceptable salt thereof (e.g., Crystalline Form A of L-malate Salt of Compound 1, Crystalline Form A of L-tartrate Salt of Compound 1), or a pharmaceutical composition described herein is administered to the subject every 8 hours.

In certain instances, a patient's condition does not improve or does not significantly improve following administration of the crystalline forms of Compound 1, a pharmaceutical salt thereof, or a pharmaceutical composition described herein and, upon the doctor's discretion the administration of the crystalline forms of Compound 1, a pharmaceutical salt thereof, or a pharmaceutical composition described herein is optionally administered chronically, that is, for an extended period of time, including throughout the duration of the patient's life in order to ameliorate or otherwise control or limit the symptoms of the patient's disease or condition.

In certain cases wherein the patient's status does improve or does not substantially improve, upon the doctor's discretion the administration of the crystalline forms of Compound 1, a pharmaceutical salt thereof, or a pharmaceutical composition described herein are optionally given continuously; alternatively, the dose of drug being administered is optionally temporarily reduced or temporarily suspended for a certain length of time (i.e., a “drug holiday”). In some or any embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes a reduction from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some or any embodiments, once improvement of the patient's conditions has occurred, a maintenance dose is administered if necessary. In some embodiments, the dosage, e.g., of the maintenance dose, or the frequency of administration, or both, are reduced, as a function of the symptoms, to a level at which the improved disease, disorder or condition is retained. In some or any embodiments, however, patients are optionally given intermittent treatment on a long-term basis upon any recurrence of symptoms.

In some or any embodiments, the amount of a given agent that corresponds to an effective amount varies depending upon factors such as the particular compound, disease or condition and its severity, the identity (e.g., weight) of the subject or host in need of treatment. In some embodiments, the effective amount is, nevertheless, determined according to the particular circumstances surrounding the case, including, e.g., the specific agent that is administered, the route of administration, the condition being treated, and the subject or host being treated. In some or any embodiments, however, doses employed for adult human treatment is in the range of about 0.02 to about 5000 mg per day, in a specific embodiment about 1 to about 1500 mg per day. In various embodiments, the desired dose is conveniently presented in a single dose or as divided doses administered simultaneously (or over a short period of time) or at appropriate intervals, for example as two, three, four or more sub-doses per day.

In some or any embodiments, the daily dosages appropriate for the crystalline forms of Compound 1, a pharmaceutical salt thereof, or a pharmaceutical composition described herein are from about 0.01 to about 2.5 mg/kg per body weight. In some embodiments, an indicated daily dosage in the larger subject, including, but not limited to, humans, is in the range from about 0.5 mg to about 100 mg, conveniently administered in divided doses, including, but not limited to, up to four times a day or in extended release form. In some or any embodiments, suitable unit dosage forms for oral administration comprise from about 1 to about 50 mg active ingredient. The foregoing ranges are merely suggestive, as the number of variables in regard to an individual treatment regime is large, and considerable excursions from these recommended values are not uncommon. In some or any embodiments, the dosages are altered depending on a number of variables, not limited to the activity of the compound used, the disease or condition to be treated, the mode of administration, the requirements of the individual subject, the severity of the disease or condition being treated, and the judgment of the practitioner.

In some or any embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. In some or any embodiments, compounds exhibiting high therapeutic indices are preferred. In some embodiments, the data obtained from cell culture assays and animal studies is used in formulating a range of dosage for use in human. In specific embodiments, the dosage of such compounds lies within a range of circulating concentrations that include the ED₅₀ with minimal toxicity. In some or any embodiments, the dosage varies within this range depending upon the dosage form employed and the route of administration utilized.

### XII. Kits/Articles of Manufacture

Articles of manufacture, comprising packaging material, crystalline forms of Compound 1, a pharmaceutical salt thereof, or a pharmaceutical composition described herein that is effective for modulating the activity of the enzyme GCS, or for treatment, prevention or amelioration of one or more symptoms of a GCS-mediated disease, disorder or condition, within the packaging material, and a label that indicates that the crystalline forms of Compound 1, a pharmaceutical salt thereof, or a pharmaceutical composition described herein is used for modulating the activity of GCS, or for treatment, prevention or amelioration of one or more symptoms of GCS-mediated disease, disorder or condition, are provided.

For use in the therapeutic applications described herein, kits and articles of manufacture are also described herein. In various embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

In some embodiments, the articles of manufacture described herein contain packaging materials. Packaging materials for use in packaging pharmaceutical products include, but are not limited to, blister packs, bottles, tubes, inhalers, pumps, bags, vials, containers, syringes, bottles, and any packaging material suitable for a selected formulation and intended mode of administration and treatment.

In some embodiments, the container(s) described herein comprise one or more crystalline forms of Compound 1, or a pharmaceutical salt thereof described herein, optionally in a composition or in combination with an additional therapeutic agent as disclosed herein. The container(s) optionally have a sterile access port (for example in some embodiments the container is an intravenous solution bag or a vial having a stopper pierceable by a hypodermic injection needle). Such kits optionally comprise a crystalline form of Compound 1, a pharmaceutical salt thereof, or a pharmaceutical composition described herein with an identifying description or label or instructions relating to its use in the methods described herein.

In some embodiments, a kit will comprises one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions is optionally included.

In some or any embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some or any embodiments, a label indicates that the contents are to be used for a specific therapeutic application. In some embodiments, the label indicates directions for use of the contents, such as in the methods described herein.

In some or any embodiments, the pharmaceutical compositions are presented in a pack or dispenser device which contains one or more unit dosage forms containing the crystalline forms of Compound 1, or a pharmaceutical salt thereof described herein. In some embodiments, the pack contains a metal or plastic foil, such as a blister pack. The pack or dispenser device is optionally accompanied by instructions for administration. In some embodiments, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. In some or any embodiments, such notice is, for example, the labeling approved by the U.S. Food and Drug Administration for prescription drugs, or the approved product insert. In some embodiments, compositions containing the crystalline forms of Compound 1, or a pharmaceutical salt thereof described herein are formulated in a compatible pharmaceutical carrier and are placed in an appropriate container labeled for treatment of an indicated condition.

Any combination of the groups described above for the various variables is contemplated herein.

### EXAMPLES

For the purpose of illustration, the following examples are included. However, it is to be understood that these examples do not limit the invention and are only meant to suggest a method of practicing the present disclosure. Persons skilled in the art will recognize that the chemical reactions described may be readily adapted to prepare a number of other compounds and crystalline forms of the present disclosure, and alternative methods for preparing the compounds and crystalline forms thereof of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds and crystalline forms thereof of the present disclosure.

For illustrative purposes, the following shows general synthetic schemes for preparing the compounds and crystalline forms thereof of the present disclosure as well as key intermediates. Those skilled in the art will appreciate that other synthetic schemes may be used to synthesize the compounds and crystalline forms thereof. Although specific starting materials and reagents are depicted and discussed below, other starting materials and reagents can be easily substituted to provide a variety of derivatives and/or reaction conditions.

Unless otherwise specified, the Crystalline Form A, Crystalline Form B, Crystalline Form C, Crystalline Form D, and Crystalline Form of Compound 1 described in the specification and examples refer to the Crystalline Form A of Compound 1, Crystalline Form B of Compound 1, Crystalline Form C of Compound 1, Crystalline Form D of Compound 1, and Crystalline Form E of Compound 1, respectively.

The following abbreviations have been used in the examples:

| Acronyms | Full Name |
|---|---|
| DMSO | dimethyl sulfoxide |
| DIPEA | diisopropylethylamine |
| DCM | dichlorormethane |
| DMF | dimethylformamide |
| EDCI | 1-ethyl-3-(3-dimethylaminopropyl)carbodiimide |
| TBDMS | tert-butyldimethylsilyl |
| TBAF | tetra-n-butylammonium fluoride |
| THF | tetrahydrofuran |
| HATU | O-(7-azabenzotriazol-1-yl)-N,N,N′,N′- |
| | tetramethyluronium hexafluorophosphate |
| MeOH | methanol |
| aq | aqueous |
| a.w. | activity of water or water activity |
| v/v | volume/volume |

### Preparation of Compound 1

The synthesis of Compound 1 is described in detail at Example 173 of WO2015042397A1, which is reproduced below.

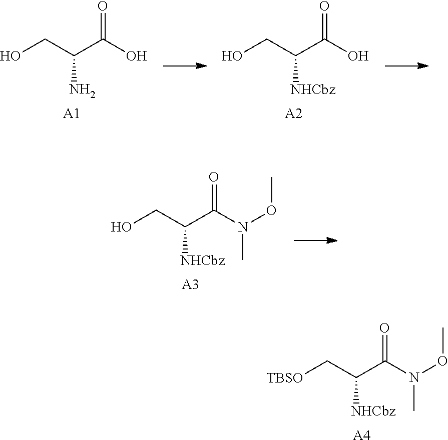

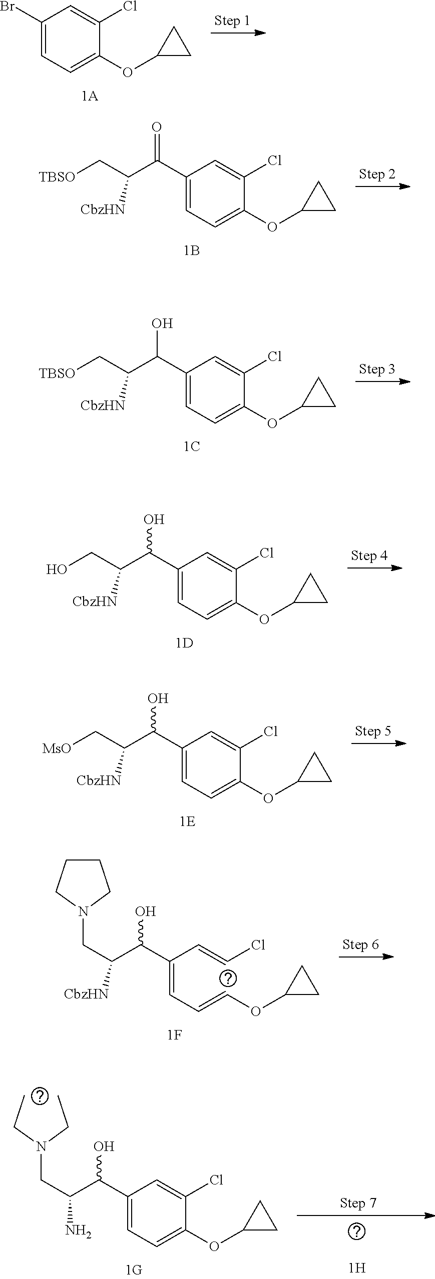

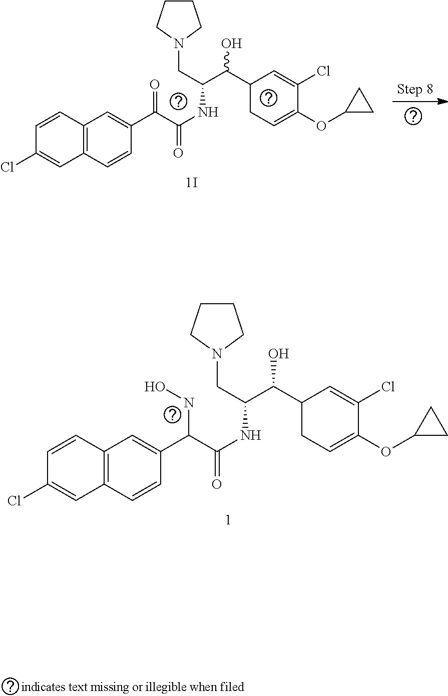 [SMILES: O=C(N[C@H](CN1CCCC1)C(O)C1C=C(Cl)C(OC2CC2)=CC1)C(=O)c1ccc2cc(Cl)ccc2c1 ; O=C(N[C@H](CN1CCCC1)[C@H](O)C1C=C(Cl)C(OC2CC2)=CC1)C(NO)c1ccc2cc(Cl)ccc2c1]

### Synthesis of Compound A4

Benzyl chloroformate (50 mL, 50 w.t. % solution in toluene, 148 mmol) was added to a solution of (R)-2-amino-3-hydroxypropanoic acid (Compound A1) (10.5 g, 100 mmol) in sat. aq NaHCO₃ solution (400 mL). The mixture was stirred vigorously for 4 h at 20° C., and the aqueous solution was extracted with ether (400 mL×2). The aqueous phase was acidified with conc. hydrochloric acid to pH=2 and extracted with ethyl acetate (300 mL×3). The combined organic phase was dried with Na₂SO₄ and concentrated to afford crude product Compound A2 (20 g, yield 84%) as a white solid. LC-MS (m/z): 240 [M+1]⁺; H-NMR (DMSO-d₆, 400 MHz) peaks: δ (ppm) 3.653 (m, 2H), 4.051 (m, 1H), 4.884 (m, 1H), 5.038 (s, 2H), 7.303-7.373 (m, 6H), 12.658 (s, 1H).

To a mixture of EDCI HCl (2.4 g, 12.5 mmol), HOBt (1.7 g, 12.5 mmol), DIPEA (2.7 g, 20 mmol) in DCM (50 mL) was added Compound A2 (1 g, 4 mmol) and N,O-dimethylhydroxylamine hydrochloride (1.2 g, 12.5 mmol). The mixture was stirred at room temperature overnight. The mixture was washed with hydrochloric acid solution (1 M, 50 mL×2), saturated aqueous NaHCO₃ (20 mL), brine (20 mL), and dried over Na₂SO₄. The crude product was purified by silica gel column chromatography (ethyl acetate in petroleum, 30% v/v) to give Compound A3 (826 mg, yield 70%) as a colorless liquid. LC-MS (m/z): 283 [M+1]⁺; ¹H-NMR (CDCl₃, 400 MHz) peaks: δ (ppm) 3.113 (s, 3H), 3.673 (s, 3H), 3.743 (t, J=4.8 Hz, 2H), 4.766 (m, 1H), 4.959-5.044 (m, 2H), 6.046 (d, J=8.0 Hz, 1H), 7.200-7.254 (m 5H).

TBDMS-Cl (800 mg, 5.31 mmol) in THF (10 mL) was added dropwise to a solution of Compound A3 (500 mg, 1.77 mmol) and imidazole (602 mg, 8.86 mmol) in THF (20 mL) at 0° C. The mixture was stirred at rt for 2 h, and then filtered. The filtrate was washed with IN HCl (50 mL×2) and brine (50 mL), and dried over Na₂SO₄. The crude product was purified with silica gel column chromatography (ethyl acetate in petroleum, 13% v/v) to give Compound A4 (526 mg, yield 75%) as a colorless liquid. LC-MS (m/z): 396 [M+1]⁺; ¹H-NMR (CDCl₃, 400 MHz) peaks: δ (ppm) 0.012 (s, 3H), 0.085 (s, 6H), 0.852 (s, 9H), 3.211 (s, 3H), 3.756 (s, 3H), 3.794-3.896 (m, 2H), 4.809 (m, 1H), 5.085 (q, J=11.2 Hz, 2H), 5.662 (d, J=8.8 Hz, 1H), 7.286-7.351 (m 5H).

### Step 1

To a solution of Compound 1A (3.73 g, 15.1 mmol) in THF (200 mL) was added n-BuLi (6.1 mL) at −60° C. under N₂ and it was stirred for 0.5 h, and then added slowly Compound A4 (2 g, 5.06 mmol) in THF (10 mL). The mixture was stirred at −60° C. for 1 h. After the addition of saturate aq NH₄Cl solution, the mixture was extracted with ethyl acetate (100 mL×2), brine (100 mL), and dried over Na₂SO₄. The crude product was purified by column chromatography on silica gel (ethyl acetate in petroleum, 10% v/v) to yield Compound 1B (1.2 g, yield 47%) as a colorless oil. LC-MS (m/z): 504.2 [M+1]⁺.

### Step 2

Compound 1B (714 mg, 1.42 mmol) was dissolved in THF (9 mL) and cooled down −80° C. under nitrogen atmosphere. L-Selectride (3 mL of 1M solution in THF, 18.9 mmol) was added dropwise while keeping the temperature at −80° C. After an hour, the reaction was quenched with saturate aq NH₄Cl solution and extracted with ethyl acetate (50 mL×2), washed with brine (100 mL), and dried over Na₂SO₄. The crude product was purified by column chromatography on silica gel (10% ethyl acetate in petroleum) to give Compound 1C (350 g, yield 49%) as a colorless oil. LC-MS (m/z): 488.2 [M-17]⁺.

### Step 3

A solution of Compound 1C (2.08 g, 4.11 mmol) in THF (40 mL) was added TBAF (537 mg, 2.06 mmol) at 0° C. and the mixture was stirred at room temperature overnight. The mixture was condensed and then added water (50 mL). It was extracted with ethyl acetate (50 mL×2), washed with brine (100 mL), dried over Na₂SO₄, and concentrated. The residue was suspended in a mixture solution of ethyl acetate in petroleum (10% v/v). Filtration gave Compound 1D (1 g, yield 65%) as a white solid. LC-MS (m/z): 374.1 [M+1]⁺.

### Step 4

A solution of Compound 1D (0.9 g, 2.3 mmol) in THF (60 mL) was added Et₃N (700 mg, 6.9 mmol) and the mixture was cooled to −15° C., to the mixture was added MsCl (467 mg, 4.1 mmol) slowly. The mixture was stirred at −15° C. about half an hour, and then diluted water. It was extracted with ethyl acetate (50 mL×2), washed with brine (50 mL), dried over Na₂SO₄, and concentrated to give Compound 1E (1.12 g, crude), which was used for the next step without further purification. LC-MS (m/z): 452.1 [M+1]⁺.

### Step 5

To a solution of Compound 1E (1.12 g, 2.83 mmol) in THF (60 mL) was added pyrrolidine (2 g, 28 mmol). The reaction mixture was allowed to warm up to room temperature and then heated at 50° C. overnight. The crude product was purified by column chromatography on silica gel (methanol in dichloromethane, 5% v/v) to render Compound IF (0.5 g, yield 50%). LC-MS (m/z): 445 [M+1]⁺.

### Step 6

To a solution of Compound IF (520 mg, 1.17 mmol) in ethanol (12 mL) and water (2 mL) was added LiOHH₂O (197 mg, 4.68 mmol). The mixture was stirred at 80° C. overnight, diluted water, extracted with DCM (50 mL×2), washed with brine (50 mL), dried over Na₂SO₄, and concentrated to give Compound 1G (360 mg, crude), which was used for the next step without further purification. LC-MS (m/z): 311.1 [M+1]⁺.

### Step 7

A mixture of Compound 1H (137 mg, 0.59 mmol), HATU (445 mg, 1.17 mmol), DMF (4 mL), and Compound 1G (218 mg, 0.70 mmol) in DCM (16 mL) was stirred at room temperature overnight. The reaction mixture was treated with water (20 mL), extracted with DCM (50 mL×2), washed with brine (50 mL), dried over anhydrous sodium sulfate, and concentrated. The crude product was purified with prep-HPLC to afford Compound 1I. LC-MS (ESI) m/z: 527 [M+H]⁺; ¹H-NMR (CD3OD, 400 MHz): δ (ppm) 0.54-0.79 (m, 4H), 2.04-2.22 (m, 5H), 3.23-3.26 (m, 1H), 3.58-3.82 (m, 6H), 4.70-4.73 (m, 1H), 4.99 (d, J=2.8 Hz, 1H), 7.30 (d, J=8.4 Hz, 1H), 7.37-7.41 (m, 1H), 7.50 (d, J=2.0 Hz, 1H), 7.57-7.59 (m, 1H), 7.88-7.93 (m, 3H), 7.99 (d, J=1.2 Hz, 1H), 8.29 (s, 1H).

### Step 8

A mixture of Compound 11 (40.8 mg, 0.08 mmol), hydroxylamine hydrochloride (76 mg, 1.1 mmol) and methanol (9 mL) was stirred at 50° C. overnight. Then the mixture was purified with prep-HPLC to provide Compound 1. LC-MS (ESI) m/z: 542.1 [M+H]f; ¹H-NMR (MeOD, 400 MHz) δ (ppm) 0.69-0.84 (m, 4H), 2.02-2.22 (m, 4H), 3.21-3.29 (m, 1H), 3.62-3.80 (m, 5H), 4.70-4.73 (m, 1H), 5.01 (d, J=2.8 Hz, 1H), 7.32-7.34 (m, 1H), 7.39-7.43 (m, 1H), 7.45-7.49 (m, 1H), 7.51-7.53 (m, 1H), 7.63-7.85 (m, 3H), 7.88-7.90 (m, 1H).

### General Procedures

Generally, XRPD analysis, DSC analysis, TGA analysis, PLM analysis, Karl Fischer (KF) analysis, and ¹H NMR analysis were used in the examples below. The instruments and conditions for each analysis were listed as follows.

X-ray Powder Diffractometer (XRPD)

| | |
|---|---|
| Instrument | Bruker D8 Advance |
| Detector | LYNXEYE_XE_T(1D mode) |
| Open angle | 2.94° |
| Scan mode | Continuous PSD fast |
| Radiation | Cu/K-Alpha1 (λ = 1.5418 \acute{Å}) |
| X-ray generator | 40 kV, 40 mA |
| power | |
| Step size | 0.02° |
| Time per step | 0.3 second per step |
| Scan range | 2°to 40° |
| Primary beam | Twin_Primary motorized slit 10.0 mm by sample |
| path slits | length; SollerMount axial soller 2.5° |
| Secondary beam | Detector OpticsMount soller slit 2.5°, |
| path slits | Twin_Secondary motorized slit 5.2 mm |
| Sample rotation | 15 rpm |
| speed | |

Differential Scanning Calorimetric (DSC)

| | |
|---|---|
| Instrument | TA Discovery 2500 or Q2000 |
| Sample pan | Tzero pan and Tzero hermetic lid with |
| | a pin hole |
| Temperature range | 30 to 250° C. or before |
| | decomposition |
| Heating rate | 10° C./min |
| Nitrogen flow | 50 mL/min |
| Sample mass | ~0.5-2 mg |

Thermal Gravimetric Analysis (TGA)

| | |
|---|---|
| Instrument | TA Discovery 5500 or Q5000 |
| Sample pan | Aluminum, open |
| Nitrogen flow | Balance 10 mL/min; sample 25 mL/min |
| Start temperature | Ambient condition (below 35° C.) |
| Final temperature | 300° C. or abort next segment if |
| | weight <80% (w/w) (The weight loss of the |
| | compound is no more than 20% (w/w)) |
| Heating rate | 10° C./min |
| Sample mass | ~2-10 mg |

Polarized Light Microscope (PLM)

| | |
|---|---|
| Instrument | OLYMPUS BX53LED |
| Method | Crossed polarizer, silicone oil added |

Karl Fischer

| | |
|---|---|
| Instrument | Mettler Toledo Coulometric KF Titrator |
| | C30 |
| Method | Coulometric |

Nuclear Magnetic Resonance (NMR)

| | |
|---|---|
| Instrument | Bruker Avance-AV 400M |
| Frequency | 400 MHz |
| Probe | 5 mm PABBO BB-1H/D |
| Number of scan | 8 |
| Temperature | 297.6 K |
| Relaxation delay | 1 second |

Dynamic Water Sorption (DVS)

| | |
|---|---|
| Instrument | SMS Intrinsic, Intrinsic Plus, Advantage or |
| | Adventure |
| Total gas flow | 200 sccm |
| Oven temperature | 25° C. |
| Solvent | Water |
| Method | Cycle: 40-0-95-0-40% RH |
| | Stage Step: 10% RH |
| | Equilibrium: 0.002 dm/dt (%/min) |
| | Minimum dm/dt stability duration: 60 min |
| | Maximum dm/dt stage time: 360 min |
| Sample mass | ~5-50 mg |

### Example 1: Preparation, Characterization and Evaluation of Crystalline Form a of Compound 1

### Preparation of Crystalline Form a of Compound 1

### (1) Equilibration Experiment at 25° C.

About 50 mg of crystalline material was equilibrated in suitable amounts of different solvents (as shown in Table 1 below) at 25° C. for 2 weeks with a stirring plate. The obtained suspensions were filtered. The solid parts (wet cakes) were investigated by XRPD. Each of the following solvents used in Table 1 provided Crystalline Form A of Compound 1.

TABLE 1
Equilibration with different solvents at 25° C. for 2 weeks

| Example No. | Solvent |
|---|---|
| 1.1 | Water |
| 1.2 | Ethanol |
| 1.3 | 2-Propanol |
| 1.4 | Acetone |
| 1.5 | Methyl ethyl ketone |
| 1.6 | Acetonitrile |
| 1.7 | Ethyl acetate |
| 1.8 | Isopropyl acetate |
| 1.9 | 1,4-Dioxane |
| 1.10 | Tetrahydrofuran |
| 1.11 | Dichloromethane |
| 1.12 | Methanol/water (40:60, v/v) a.w. 0.8 |
| 1.13 | Acetone/water (50:50, v/v) a.w. 0.9 |
| 1.14 | Acetonitrile/water (90:10, v/v) a.w. 0.9 |

### (2) Equilibration Experiment at 50° C.

About 50 mg of the crystalline material was equilibrated in suitable amounts of different solvents (as shown in Table 2 below) at 50° C. for 1 week with a stirring plate. The obtained suspensions were filtered. The solid parts (wet cakes) were investigated by XRPD. Each of the following solvents used in Table 2 provided Crystalline Form A of Compound 1.

TABLE 2
Equilibration with different solvents at 50° C. for 1 week

| Example No. | Solvent |
|---|---|
| 1.15 | Water |
| 1.16 | Ethanol |
| 1.17 | 2-Propanol |
| 1.18 | Acetone |
| 1.19 | Methyl ethyl ketone |
| 1.20 | Acetonitrile |
| 1.21 | Ethyl acetate |
| 1.22 | Isopropyl acetate |
| 1.23 | 1,4-Dioxane |
| 1.24 | Tetrahydrofuran |
| 1.25 | Toluene |
| 1.26 | Methanol/water (40:60, v/v) a.w. 0.8 |
| 1.27 | Acetone/water (50:50, v/v) a.w. 0.9 |
| 1.28 | Acetonitrile/water (90:10, v/v) a.w. 0.9 |

### (3) Anti-Solvent Experiment

30 mg-50 mg of the crystalline material was dissolved in different solvents (as shown in Table 3 below). The obtained solutions were filtered by 0.45 μm nylon filter to get clear solutions. Each of the anti-solvents was added into the clear solutions slowly. Precipitates were collected by filtration. The solid parts (wet cakes) were investigated by XRPD. Each of the solvent/anti-solvent systems used in Table 3 provided Crystalline Form A of Compound 1.

TABLE 3
Precipitation by addition of anti-solvent

| Example | | Anti- | | |
|---|---|---|---|---|
| No. | Solvent | solvent | XRPD | Comments |
| 1.29 | Ethanol | Heptane | Crystalline | Stirring at 25° C. |
| | | (3V) | Form A of | for 1 week |
| | | | Compound 1 | |
| 1.30 | Ethanol | Water | Crystalline | No form change |
| | | (1V) | Form A of | was observed |
| | | | Compound 1 | after stirring at |
| | | | | 25° C. for 1 week |
| 1.31 | Acetone | Heptane | Crystalline | No form change |
| | | (3V) | Form A of | was observed |
| | | | Compound 1 | after stirring at |
| | | | | 25° C. for 1 week |
| 1.32 | Acetone | Water | Crystalline | No form change |
| | | (1V) | Form A of | was observed |
| | | | Compound 1 | after stirring at |
| | | | | 25° C. for 1 week |
| 1.33 | Acetone | Acetonitrile | Crystalline | Stirring at 25° C. |
| | | (3V) | Form A of | for 1 week |
| | | | Compound 1 | |
| 1.34 | Acetone | Ethyl acetate | Clear | Stirring at 25° C. |
| | | (3V) | solution | for 1 week |
| 1.35 | 1,4-Dioxane | Heptane | Crystalline | No form change |
| | | (3V) | Form A of | was observed |
| | | | Compound 1 | after stirring at |
| | | | | 25° C. for 1 week |
| 1.36 | 1,4-Dioxane | Water | Crystalline | Stirring at 25° C. |
| | | (2V) | Form A of | for 1 week |
| | | | Compound 1 | |

Note:
“V” refers to the volume ratio of anti-solvent and solvent.

### (4) Crystallization at Room Temperature by Slow Evaporation

The crystalline material was dissolved in suitable amount of acetone/acetonitrile (1:1, v/v). The obtained solution was filtered by 0.45 μM nylon filter. Obtained clear solution was slowly evaporated at ambient condition. Solid residues were investigated by XRPD and identified as Crystalline Form A of Compound 1.

### (5) Fast Cooling

30 mg˜50 mg of the crystalline material was dissolved in the minimal amounts of selected solvents (as shown in Table 4 below) at 50° C. The obtained solutions were put into an ice bath and agitated. Precipitates were collected by filtration. The solid parts (wet cakes) were investigated by XRPD. Each of the following solvents provided Crystalline Form A of Compound 1: ethanol, acetone, methyl ethyl ketone, and 1,4-dioxane. Using this method, tetrahydrofuran, dichloromethane, acetonitrile/tetrahydrofuran (1:1, v/v), and ethyl acetate/tetrahydrofuran (1:1, v/v) did not provide Crystalline Form A of Compound 1. When no precipitation was obtained, the solutions were put in −20° C. freezer for crystallization.

TABLE 4
Crystallization from hot saturated solutions by fast cooling

| Example No. | Solvents | XRPD | Comments |
|---|---|---|---|
| 1.37 | Ethanol | Crystalline | Precipitated at 0° C. |
| | | Form A of | |
| | | Compound 1 | |
| 1.38 | Acetone | Crystalline | Precipitated at 5° C. |
| | | Form A of | |
| | | Compound 1 | |
| 1.39 | Methyl ethyl | Crystalline | Precipitated at 5° C. |
| | ketone | Form A of | |
| | | Compound 1 | |
| 1.40 | 1,4-Dioxane | Crystalline | Precipitated at 5° C. |
| | | Form A of | |
| | | Compound 1 | |
| 1.41 | Tetrahydrofuran | Clear solution | Stirring at 5° C. for |
| | | | 5 days, then keep |
| | | | at −20° C. for |
| | | | 5 days |
| 1.42 | Dichloromethane | Clear solution | Stirring at 5° C. for |
| | | | 5 days, then keep |
| | | | at −20° C. for |
| | | | 5 days |
| 1.43 | Acetonitrile/ | Clear solution | Stirring at 5° C. for |
| | tetrahydrofuran | | 5 days, then keep |
| | (1:1, v/v) | | at −20° C. for |
| | | | 5 days |
| 1.44 | Ethyl acetate/ | No sufficient | Stirring at 5° C. for |
| | tetrahydrofuran | solids | 5 days, then keep |
| | (1:1, v/v) | | at −20° C. for |
| | | | 5 days |

### Characterization of Crystalline Form a of Compound 1

### (1) XRPD Data

The XRPD pattern for Crystalline Form A of Compound 1 is shown in FIG. 1. The peaks and their relative intensities in the XRPD pattern are shown in Table 5 below.

TABLE 5
Peaks in the XRPD Pattern for Crystalline Form A of Compound 1

| Peak Position [°2θ] | Relative Intensity (%) |
|---|---|
| 18.883 | 100 |
| 10.889 | 96.6 |
| 21.309 | 79.4 |
| 14.974 | 73.4 |
| 24.160 | 61.8 |
| 18.663 | 60.9 |
| 17.109 | 51.7 |
| 7.518 | 46.5 |
| 20.950 | 41.1 |
| 19.602 | 40.9 |
| 27.784 | 40.1 |
| 28.281 | 40 |
| 17.494 | 37.6 |
| 14.743 | 34.5 |
| 22.386 | 34.1 |
| 31.979 | 33.3 |
| 21.759 | 28.9 |
| 25.017 | 28.3 |
| 30.623 | 26.7 |
| 10.301 | 24.8 |
| 26.760 | 23.6 |
| 21.536 | 16.7 |
| 19.337 | 15.4 |
| 30.103 | 14 |
| 33.310 | 13.8 |
| 31.467 | 13.7 |
| 23.309 | 13.4 |
| 25.781 | 13.1 |
| 27.362 | 10.6 |
| 35.751 | 9.8 |
| 23.835 | 9.6 |
| 23.601 | 7.8 |
| 37.946 | 7 |
| 33.960 | 6.9 |
| 39.178 | 6.9 |
| 10.569 | 5.2 |
| 36.533 | 5.2 |
| 26.283 | 5.1 |
| 29.254 | 4.1 |
| 28.891 | 3.9 |
| 34.526 | 3.9 |
| 17.884 | 3.4 |
| 13.697 | 3.2 |
| 32.877 | 2.4 |
| 36.134 | 1.8 |
| 11.644 | 1.7 |
| 37.542 | 1.5 |
| 16.364 | 1.4 |
| 14.156 | 0.9 |
| 35.228 | 0.9 |

### (2) TGA and DSC Data

The TGA curve and DSC curve for Crystalline Form A of Compound 1 are shown in FIG. 2 and FIG. 3, respectively. As shown in FIG. 2, about 0.6% weight loss was observed between about 24° C. and about 200° C., suggesting that Crystalline Form A of Compound 1 is an anhydrous crystalline form. As shown in FIG. 3, Crystalline Form A of Compound 1 has a melting onset of 206.9° C. (97 J/g), and decomposition occurred upon melting.

### (3) ¹H NMR Data

The ¹H NMR spectrum for Crystalline Form A of Compound 1 is shown in FIG. 4. As shown in FIG. 4, no residual solvent was detected by ¹H NMR.

### (4) KF Data

KF analysis showed that Crystalline Form A of Compound 1 contained about 0.12% water by weight.

### Evaluation of Crystalline Form A of Compound 1

### (1) Stability

Crystalline Form A of Compound 1 is chemically and physically stable at 25° C./92% RH in an open container for one week, at 40° C./75% RH in an open container for one week, and at 60° C. in a tight container for one week (see Table 20 in Example 12 below). In view of the stability evaluation results above, Crystalline Form A of Compound 1 is highly stable.

### (2) Hygroscopicity

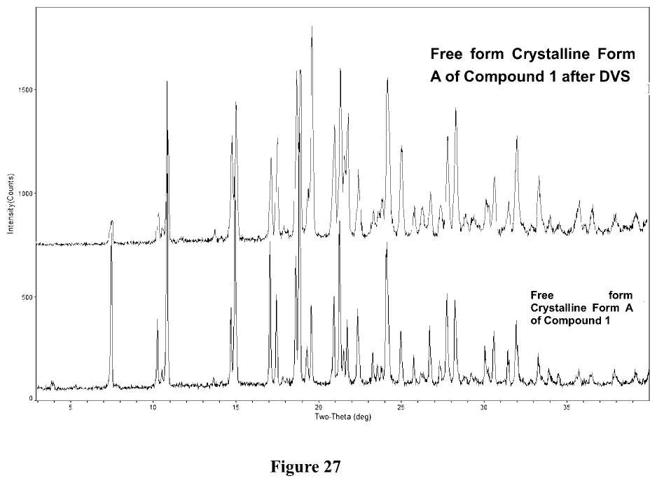

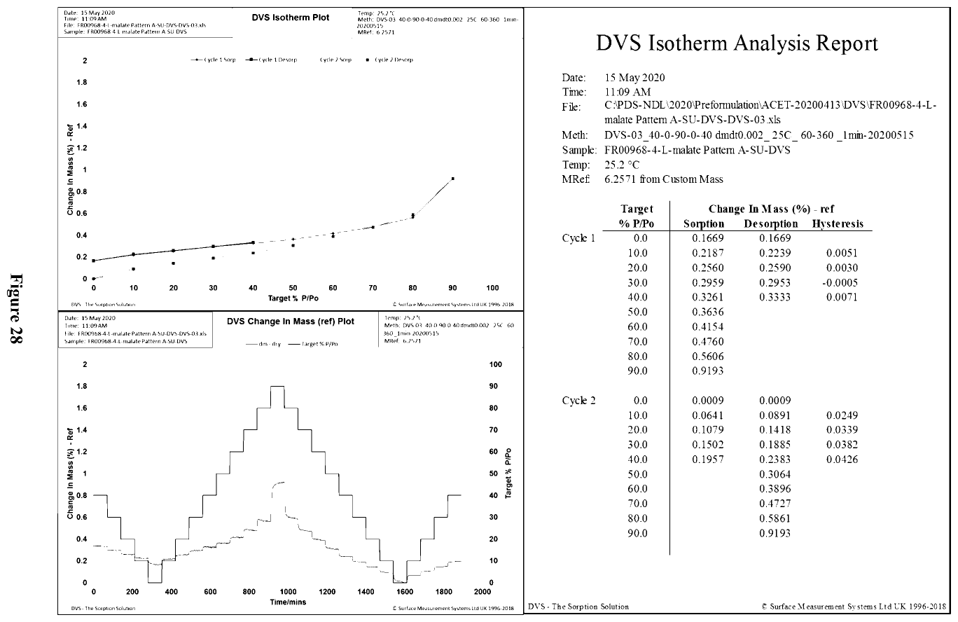

The hygroscopicity of Crystalline Form A of Compound 1 was evaluated by DVS test. The DVS diagram of Crystalline Form A of Compound 1 is shown in FIG. 26. According to the DVS test results, water uptake increased from 0.06% to 0.39% when relative humidity was raised from 40% RH to 90% RH at 25° C. in DVS study, suggesting that Crystalline Form A of Compound 1 is slightly hygroscopic. No form change was observed after DVS test, as evidenced by the XRPD patterns of Crystalline Form A of Compound 1 before and after DVS test shown in FIG. 27. All the data suggested that Crystalline Form A of Compound 1 is an anhydrate of high crystallinity.

### Example 2: Preparation and Characterization of Crystalline Form B of Compound 1

### Preparation of Crystalline Form B of Compound 1

### (1) Equilibration Experiment at 25° C.

Crystalline Form A of Compound 1 was used in this method. About 50 mg of the crystalline material was equilibrated in suitable amount of methanol at 25° C. for 2 weeks with a stirring plate. Obtained suspension was filtered. The solid part (wet cake) was characterized by XRPD and identified as Crystalline Form B of Compound 1.

### (2) Equilibration Experiment at 50° C.

Crystalline Form A of Compound 1 was used in this method. About 50 mg of the crystalline material was equilibrated in suitable amount of methanol at 50° C. for 1 week with a stirring plate. Obtained suspension was filtered. The solid part (wet cake) was characterized by XRPD and identified as Crystalline Form B of Compound 1.

### (3) Scale Up Experiment of Crystalline Form B of Compound 1

Crystalline Form A of Compound 1 was used in this method. About 500 mg of the crystalline material was equilibrated in 10 mL of methanol at 25° C. with a stirring plate for about 5 days. The obtained solids were recovered by filtration. Solid part (wet cake) was dried at ambient condition for about 3 hours. Obtained dry solids were characterized by XRPD and identified as Crystalline Form B of Compound 1. About 447 mg Crystalline Form B of Compound 1 was obtained as a white solid with 89% yield.

### Characterization of Crystalline Form B of Compound 1

### (1) XRPD Data

The XRPD pattern for Crystalline Form B of Compound 1 is shown in FIG. 5. The peaks and their relative intensities in the XRPD pattern are shown in Table 6 below.

TABLE 6
Peaks in the XRPD Pattern for Crystalline Form B of Compound 1

| Peak Position [°2θ] | Relative Intensity (%) |
|---|---|
| 6.244 | 100 |
| 18.679 | 98.4 |
| 12.445 | 69.2 |
| 17.239 | 67 |
| 12.832 | 60.9 |
| 23.559 | 60.3 |
| 15.252 | 50.2 |
| 22.41 | 49.7 |
| 9.344 | 43.9 |
| 25.722 | 43.5 |
| 20.354 | 35.6 |
| 16.366 | 32.7 |
| 24.731 | 23.1 |
| 20.581 | 22.5 |
| 28.664 | 20.2 |
| 27.044 | 17.2 |
| 35.758 | 16.6 |
| 17.905 | 16.4 |
| 16.087 | 13.7 |
| 36.194 | 12.9 |
| 34.867 | 11.9 |
| 24.984 | 11.7 |
| 10.802 | 10.3 |
| 29.902 | 10.2 |
| 32.584 | 10.2 |
| 21.279 | 9.8 |
| 26.266 | 8.6 |
| 37.816 | 5.8 |
| 33.789 | 5.7 |
| 19.275 | 5.5 |
| 19.946 | 4.4 |
| 33.118 | 4.1 |

### (2) TGA and DSC Data

The TGA curve and DSC curve for Crystalline Form B of Compound 1 are shown in FIG. 6 and FIG. 7, respectively. As shown in FIG. 6, about 0.4% weight loss was observed between about 33° C. and 200° C. As shown in FIG. 7, Crystalline Form B of Compound 1 has multiple thermal events at onset of 178.3° C. (0.7 J/g), 179.7° C. (0.9 J/g), 196.6° C. (74 J/g), 198.7° C. (10 J/g), and 203.7° C. (22 J/g), respectively.

### (3) ¹H NMR Data

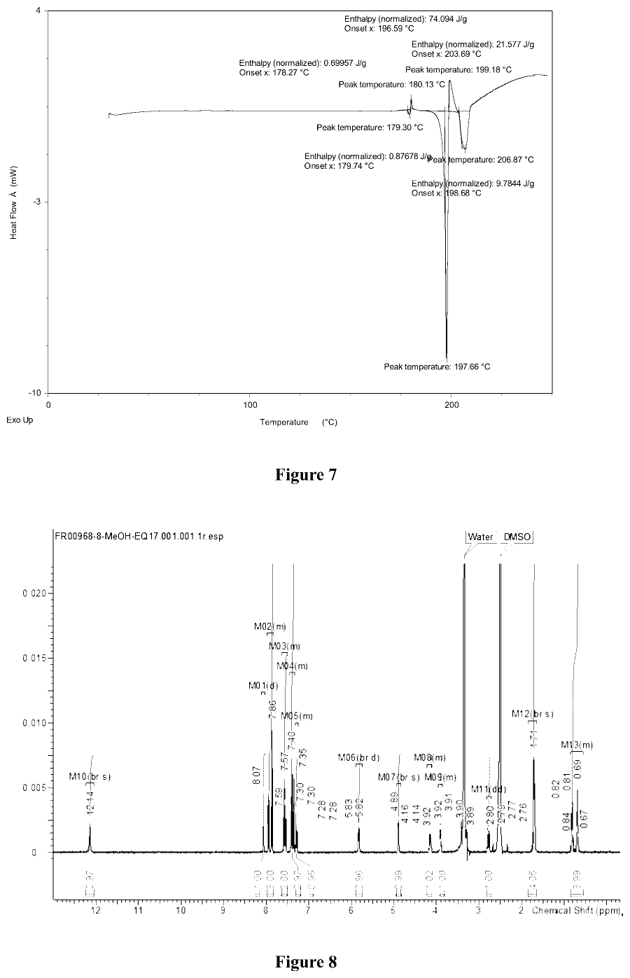

The ¹H NMR spectrum for Crystalline Form B of Compound 1 is shown in FIG. 8. As shown in FIG. 8, no residual methanol was detected by ¹H-NMR. The wet cake obtained from methanol by equilibration at 25° C. showed the same XRPD pattern as that of Crystalline Form B of Compound 1. Without air drying, about 38.8% methanol by weight (˜10 equivalent) was detected by ¹H-NMR in Crystalline Form B of Compound 1 wet cake. Based on the results, Crystalline Form B of Compound 1 is likely a methanol solvate of high crystallinity.

### Example 3: Preparation, Characterization and Evaluation of Crystalline Form C of Compound 1

### Preparation of Crystalline Form C of Compound 1

Crystalline Form A of Compound 1 was used in this method. Crystalline Form C of Compound 1 was prepared by slow evaporation experiment from methanol dichloromethane (1:1, v/v). Specifically, about 30 mg of Crystalline Form A of Compound 1 was dissolved in suitable amount of methanol dichloromethane (1:1, v/v). The obtained solutions were filtered by 0.45 μm nylon filter. Obtained clear solutions were slowly evaporated at ambient condition. Solid residues were investigated by XRPD and identified as Crystalline Form C of Compound 1.

In addition, Crystalline Form C of Compound 1 scale up experiment was performed. Specifically, about 120 mg of Crystalline Form A of Compound 1 was dissolved in 8 mL of methanol/dichloromethane (1/1, v/v). The obtained solutions were filtered by 0.45 μm nylon filter to get clear solution. Then clear solutions were fast evaporated under nitrogen flow. After drying at ambient condition for about 1 day, about 106 mg of Crystalline Form C of Compound 1 was obtained successfully as a white solid with about 87% yield. The obtained dry solids were characterized by XRPD and identified as Crystalline Form C of Compound 1.

### Characterization of Crystalline Form C of Compound 1

### (1) XRPD Data

The XRPD pattern for Crystalline Form C of Compound 1 is shown in FIG. 9. The peaks and their relative intensities in the XRPD pattern are shown in Table 7 below.

TABLE 7
Peaks in the XRPD Pattern for Crystalline Form C of Compound 1

| Peak Position [°2θ] | Relative Intensity (%) |
|---|---|
| 6.656 | 100 |
| 19.994 | 18.1 |
| 26.732 | 11.7 |
| 18.350 | 9.6 |
| 20.753 | 7.2 |
| 22.176 | 6.8 |
| 15.941 | 5.7 |
| 20.450 | 5.7 |
| 13.320 | 5.6 |
| 17.687 | 5.5 |
| 19.205 | 4.9 |
| 23.302 | 4.3 |
| 9.183 | 4 |
| 8.700 | 3.6 |
| 12.470 | 2.2 |
| 3.724 | 2.1 |
| 25.036 | 1.1 |
| 30.741 | 0.9 |
| 4.454 | 0.9 |

### (2) TGA and DSC Data

The TGA curve and DSC curve for Crystalline Form C of Compound 1 are shown in FIG. 10 and FIG. 11, respectively. As shown in FIG. 10, about 2.2% weight loss was observed at about 150° C. No obvious melting peak was observed in DSC curve (FIG. 11).

### (3) ¹H NMR Data

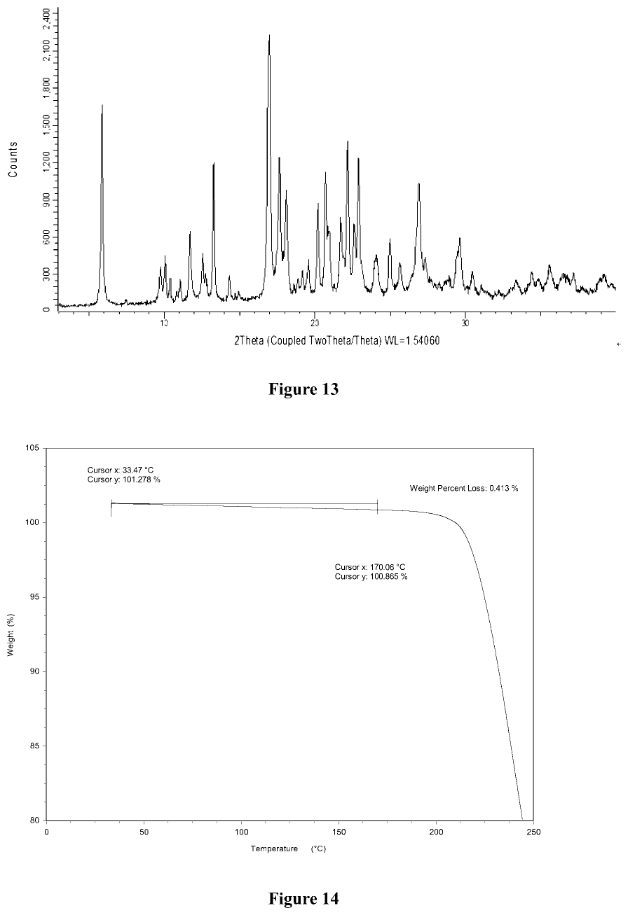

The ¹H NMR spectrum for Crystalline Form C of Compound 1 is shown in FIG. 12. As shown in FIG. 12, no residual solvent was detected by ¹H-NMR.

### (4) KF Data

KF analysis showed that Crystalline Form C of Compound 1 contained about 3.6% water by weight (1.1 equivalent), suggesting that Crystalline Form C of Compound 1 is a hydrate containing about 3.6% water by weight (1.1 equivalent).

### Evaluation of Crystalline Form C of Compound 1

See Example 6: Water Activity Study below for details.

### Example 4: Preparation, Characterization and Evaluation of Crystalline Form D of Compound 1

### Preparation of Crystalline Form D of Compound 1

About 150 mg Crystalline Form B of Compound 1 in 20 batches was heated to 190° C. by TGA, and then cooled to ambient temperature. About 110 mg light white solid (about 73% yield) was obtained. The obtained dry solids were characterized by XRPD, and identified as Crystalline Form D of Compound 1.

### Characterization of Crystalline Form D of Compound 1

### (1) XRPD Data

The XRPD pattern for Crystalline Form D of Compound 1 is shown in FIG. 13. The peaks and their relative intensities in the XRPD pattern are shown in Table 8 below.

TABLE 8
Peaks in the XRPD Pattern for Crystalline Form D of Compound 1

| Peak Position [°2θ] | Relative Intensity (%) |
|---|---|
| 16.956 | 100 |
| 5.883 | 77.8 |
| 22.181 | 58.3 |
| 17.663 | 53.5 |
| 13.291 | 53.3 |
| 22.909 | 51.7 |
| 20.718 | 45.9 |
| 26.900 | 41.2 |
| 18.113 | 40 |
| 20.205 | 32.1 |
| 11.727 | 26.3 |
| 21.756 | 25.8 |
| 22.617 | 25.7 |
| 20.956 | 22.8 |
| 29.635 | 20.3 |
| 24.990 | 20 |
| 12.566 | 17.9 |
| 10.080 | 17 |
| 24.078 | 14.7 |
| 9.761 | 13.9 |
| 19.581 | 11.5 |
| 27.280 | 11.1 |
| 25.637 | 11 |
| 12.754 | 10.2 |
| 14.335 | 9.5 |
| 35.599 | 9.1 |
| 10.411 | 8.8 |
| 30.457 | 8.6 |
| 11.067 | 8.2 |
| 19.175 | 8.1 |
| 34.373 | 7.5 |
| 36.486 | 6.4 |
| 37.185 | 6.3 |
| 39.200 | 5.6 |
| 33.344 | 5.2 |
| 18.891 | 4.2 |
| 34.841 | 4.1 |
| 10.830 | 3.7 |
| 28.859 | 3.4 |
| 14.954 | 3.3 |
| 28.254 | 2.8 |
| 31.083 | 2.6 |
| 7.456 | 1.2 |

### (2) TGA and DSC Data

The TGA curve and DSC curve for Crystalline Form D of Compound 1 are shown in FIG. 14 and FIG. 15, respectively. As shown in FIG. 14, about 0.4% weight loss was observed between about 33° C. and about 170° C. As shown in FIG. 15, Crystalline Form D of Compound 1 showed an exothermic peak at onset of 88.7° C. (8 J/g). After that, a small endothermic peak at onset of 172.8° C. (2 J/g) was observed. Then a sharp melting peak at onset of 207.4° C. with an enthalpy of about 90 J/g was observed. The data suggested that Crystalline Form D of Compound 1 is likely an anhydrate of high crystallinity.

### (3) ¹H NMR Data

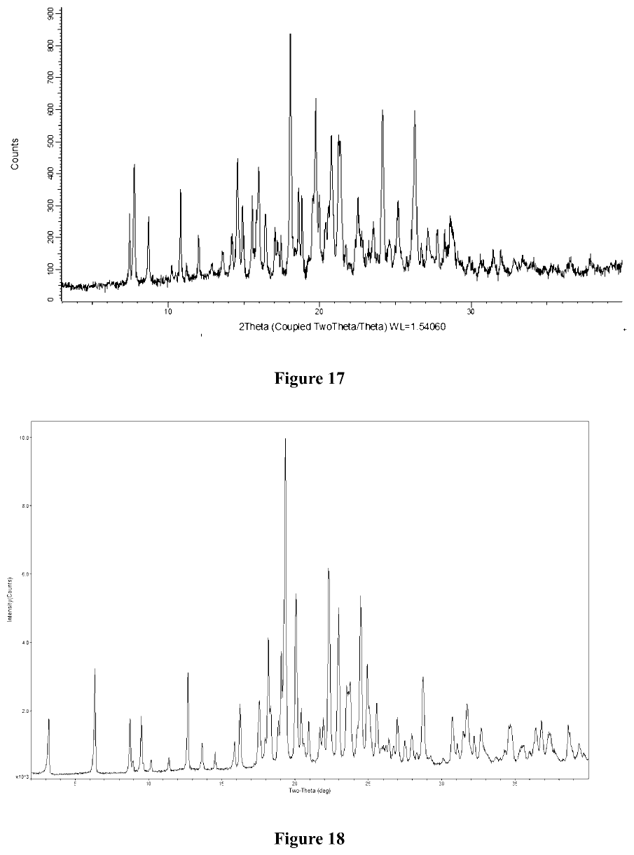

The ¹H NMR spectrum for Crystalline Form D of Compound 1 is shown in FIG. 16. As shown in FIG. 16, no residual solvent was detected by ¹H-NMR.

### Evaluation of Crystalline Form D of Compound 1

See Example 7: Competitive Equilibration Experiment below for details.

### Example 5: Preparation, Characterization and Evaluation of Crystalline Form E of Compound 1

### Preparation of Crystalline Form E of Compound 1

Crude product (18.5 g, 32.0 mmol) was purified by SFC column: Phenomenex-Cellulose-2 (250 mm*30 mm, 10 um); mobile phase: [0.1% NH₃H₂O MeOH]; B %: 60%-60%, 3.9 mins; 1770 mins) to give P1. Then the P1 were concentrated to give residue 1. EtOH (100 mL) was added to the residue 1 and obtained solution was concentrated. This work up was repeated three times to give residue 2. H₂O (100 mL) was added to the residue 2 and obtained solution was lyophilized. Crystalline Form E of Compound 1 (10.3 g, 18.5 mmol) was obtained.

### Characterization of Crystalline Form E of Compound 1

The XRPD pattern for Crystalline Form E of Compound 1 is shown in FIG. 17. The peaks and their relative intensities in the XRPD pattern are shown in Table 9 below.

TABLE 9
Peaks in the XRPD Pattern for Crystalline Form E of Compound 1

| Peak Position [°2θ] | Relative Intensity (%) |
|---|---|
| 18.089 | 100 |
| 26.315 | 76.9 |
| 19.775 | 73.2 |
| 24.177 | 62.7 |
| 20.803 | 62.1 |
| 16.018 | 49.5 |
| 21.429 | 47.5 |
| 14.608 | 46.1 |
| 21.314 | 39.3 |
| 7.794 | 37.6 |
| 20.007 | 34.3 |
| 8.744 | 32.8 |
| 15.573 | 31.9 |
| 18.636 | 29.5 |
| 28.686 | 27.3 |
| 16.43 | 25.5 |
| 25.182 | 25.5 |
| 20.584 | 25.2 |
| 10.856 | 24.7 |
| 22.515 | 22.9 |
| 19.611 | 22.3 |
| 14.233 | 20.8 |
| 22.83 | 19.4 |
| 23.557 | 19.4 |
| 27.166 | 18.2 |
| 24.647 | 17 |
| 27.764 | 15.1 |
| 17.267 | 13.9 |
| 14.962 | 13.8 |
| 18.864 | 13.3 |
| 12.049 | 12.6 |
| 17.085 | 11.7 |
| 7.486 | 11 |
| 17.477 | 10.3 |
| 13.613 | 9.1 |
| 36.59 | 8 |
| 28.293 | 7.5 |
| 21.743 | 7 |
| 31.524 | 6.9 |
| 29.879 | 6.3 |
| 34.904 | 6.1 |
| 32.851 | 5.7 |
| 35.467 | 5 |
| 31.997 | 4.9 |
| 12.878 | 4.7 |
| 11.239 | 4 |
| 30.606 | 3.8 |
| 3.691 | 3 |
| 34.151 | 2.9 |
| 33.397 | 2.8 |

### Evaluation of Crystalline Form E of Compound 1

See Example 7: Competitive Equilibration Experiment below for details.

### Example 6: Water Activity Study

Water activity experiments were conducted at 25° C. or 50° C. to determine critical water activity between anhydrate and hydrate. Saturated solutions were prepared using anhydrate Crystalline Form A of Compound 1 at 25° C. or 50° C. in 8 different water activities with ethanol/water mixtures. 4 mg of Crystalline Form A of Compound 1 and 4 mg of Crystalline Form C of Compound 1 were added to the saturated solutions. The mixtures were stirred at 25° C. or 50° C. for at least 2 days. The obtained solid part (wet cake) was investigated by XRPD.

The results of water activity study in different solvents at 25° C. and 50° C. are shown in Table 10 and Table 11, respectively. The water activity of a binary solvent system was calculated based on UNIFAC method (UNIQUAC Functional-group Activity Coefficients). As shown in Table 10 and Table 11, all the experiments result in Crystalline Form A of Compound 1, suggesting that Crystalline Form C of Compound 1 is a metastable hydrate and Crystalline Form A of Compound 1 is stable in a wide range of water activity.

TABLE 10
Water activity study at 25° C. using Crystalline
Form A and Crystalline Form C of Compound 1

| Example | | XRPD | Comments |
|---|---|---|---|
| No. | Solvents | Crystalline Form A + Crystalline Form C of | |
| Starting material | | Compound 1 | |
| 6.1 | Ethanol/water (97:3, v/v) | Crystalline Form A of | Stirring for 2 days |
| | (a.w. = 0.2) | Compound 1 | |
| 6.2 | Ethanol/water (95:5, v/v) | Crystalline Form A of | Stirring for 2 days |
| | (a.w. = 0.3) | Compound 1 | |
| 6.3 | Ethanol/water (93:7, v/v) | Crystalline Form A of | Stirring for 2 days |
| | (a.w. = 0.4) | Compound 1 | |
| 6.4 | Ethanol/water (90:10, v/v) | Crystalline Form A of | Stirring for 2 days |
| | (a.w. = 0.5) | Compound 1 | |
| 6.5 | Ethanol/water (85:15, v/v) | Crystalline Form A of | Stirring for 2 days |
| | (a.w. = 0.6) | Compound 1 | |
| 6.6 | Ethanol/water (80:20, v/v) | Crystalline Form A of | Stirring for 2 days |
| | (a.w. = 0.7) | Compound 1 | |
| 6.7 | Ethanol/water (40:60, v/v) | Crystalline Form A of | Stirring for 2 days |
| | (a.w. = 0.9) | Compound 1 | |
| 6.8 | Water (a.w. = 1.0) | Crystalline Form A of | Stirring for 2 days |
| | | Compound 1 | |

TABLE 11
Water activity study at 50° C. using Crystalline
Form A and Crystalline Form C of Compound 1

| Example | | XRPD | Comments |
|---|---|---|---|
| No. | Solvents | Crystalline Form A + Crystalline Form C of | |
| Starting material | | Compound 1 | |
| 6.9 | Ethanol/water (95:5, v/v) | Crystalline Form A of | Stirring for 2 days |
| | (a.w. = 0.3) | Compound 1 | |
| 6.10 | Ethanol/water (92:8, v/v) | Crystalline Form A of | Stirring for 2 days |
| | (a.w. = 0.4) | Compound 1 | |
| 6.11 | Ethanol/water (90:10, | Crystalline Form A of | Stirring for 2 days |
| | v/v) (a.w. = 0.5) | Compound 1 | |
| 6.12 | Ethanol/water (85:15, | Crystalline Form A of | Stirring for 2 days |
| | v/v) (a.w. = 0.6) | Compound 1 | |
| 6.13 | Ethanol/water (80:20, | Crystalline Form A of | Stirring for 2 days |
| | v/v) (a.w. = 0.7) | Compound 1 | |
| 6.14 | Ethanol/water (70:30, | Crystalline Form A of | Stirring for 2 days |
| | v/v) (a.w. = 0.8) | Compound 1 | |
| 6.15 | Ethanol/water (40:60, | Crystalline Form A of | Stirring for 2 days |
| | v/v) (a.w. = 0.9) | Compound 1 | |
| 6.16 | Water (a.w. = 1.0) | Crystalline Form A of | Stirring for 2 days |
| | | Compound 1 | |

### Example 7: Competitive Equilibration Experiment

Competitive slurry was conducted to determine thermodynamic relationships of Crystalline Form A, Crystalline Form D and Crystalline Form E of Compound 1. Saturated solutions were prepared using anhydrate Crystalline Form A of Compound 1 at 5° C., 25° C. and 50° C. in solvents, respectively. 4 mg of Crystalline Form A of Compound 1, 4 mg of Crystalline Form D of Compound 1, and 4 mg of Crystalline Form E of Compound 1 were added to the saturated solutions. The mixtures were stirred at 5° C., 25° C. or 50° C. for 3 days. Obtained solid part (wet cake) was investigated by XRPD.

The results of competitive equilibration experiment in different solvents at 5°, 25° C. and 50° C. are shown in Table 12 below. As shown in Table 12, Crystalline Form A of Compound 1 is the only product in different solvents at 5° C., 25° C. and 50° C., suggesting that Crystalline Form A of Compound 1 is the most stable form.

Overall, Crystalline Form A of Compound 1 was confirmed to be the most stable anhydrate and the optimal polymorph in these examples. Therefore, Crystalline Form A of Compound 1 is recommended for further development.

TABLE 12
Results of competitive equilibration experiment

| | | Crystalline Form A + Crystalline Form D + | | |
|---|---|---|---|---|
| | | Crystalline Form E of Compound 1 | | |
| Example | Sample | 5° C. | 25° C. | 50° C. |
| No. | Solvent | XRPD (3 days) | | |
| 7.1 | Ethanol | Crystalline | Crystalline | Crystalline |
| | | Form A of | Form A of | Form A of |
| | | Compound 1 | Compound 1 | Compound 1 |
| 7.2 | Acetone | Crystalline | Crystalline | Crystalline |
| | | Form A of | Form A of | Form A of |
| | | Compound 1 | Compound 1 | Compound 1 |
| 7.3 | Acetonitrile | Crystalline | Crystalline | Crystalline |
| | | Form A of | Form A of | Form A of |
| | | Compound 1 | Compound 1 | Compound 1 |

### Example 8: Salt Screening

Free form Crystalline Form A of Compound 1 was used as the starting material for salt screening and selection study. About 5 mg of free form Crystalline Form A of Compound 1 was weighed to a 2 mL glass vial and aliquot of 20 μL of each solvent was added to get clear solution. Max. volume of each solvent added was 1 mL. Approximate solubility of the starting material was determined by visual observation at 25° C. The approximate solubility of the starting material at 25° C. is shown in Table 13 below.

TABLE 13
Approximate solubility at 25° C.

| Example No. | Solvent | Solubility (mg/mL, 25° C.) |
|---|---|---|
| 8.1 | Ethanol | 5-5.5 |
| 8.2 | Acetone | 16.7-25 |
| 8.3 | Ethyl acetate | <5 |
| 8.4 | Acetonitrile | <5 |
| 8.5 | Water | <5 |

Based on calculated pKa of 7.85 (calculated by Marvin Sketch 5.6), 5 counter ions were selected as salt forming agents as shown in Table 14 below. Acetone and ethanol/water (95:5, v/v) were used as screening solvents. In total, about 19 salt screening experiments were conducted.

TABLE 14
Counter ions used for salt screening

| Counter ions | pKa(s) | M.W. | Class |
|---|---|---|---|
| Hydrochloric acid | −6 | 36.46 | I |
| L-malic acid | 3.4 | 134.09 | I |
| L-tartaric acid | 3.02 | 150.09 | I |
| Citric acid | 3.12 | 192.13 | I |
| Fumaric acid | 3.03 | 116.08 | I |

About 50 mg of free form Crystalline Form A of Compound 1 was added to a suitable solvent and 1.05 equivalent of counter ion was added. Liquid counter ions were diluted by 10 folds of solvents first before addition. Obtained mixtures were stirred at 50° C. for 1 hours and then at 25° C. or at least 36 hours.

For those clear solutions, the solutions were cooled to 5° C. and kept at 5° C. for at least 24 hours. If no precipitate was found, anti-solvent (ethyl acetate and heptane) was added to the clear solution. Obtained samples were stirred at 5° C. for about 7 days.

Obtained suspensions were taken out and centrifuged. Obtained solids were dried at 30-40° C. under vacuum for at least 12 hours and analyzed by XRPD. Results are summarized in Table 15 and Table 16 below. Hits with high or medium crystallinity were further characterized.

TABLE 15
Salt screening results (slurry crystallization)

| | | | B |
|---|---|---|---|
| Example | | A | Ethanol/water (95:5, |
| No. | Counter ions | Acetone | v/v) |
| 8.6 | Free form | − (Free form Crystalline Form | − (Free form |
| | | A of Compound 1) | Crystalline Form A of |
| | | | Compound 1) |
| 8.7 | Hydrochloric | Clear solution | Clear solution |
| | acid | | |
| 8.8 | L-malic acid | + (Crystalline Form A of L- | Clear solution |
| | | malate Salt of Compound 1) | |
| 8.9 | L-tartaric acid | + (Crystalline Form A of L- | + (Crystalline Form B |
| | | tartrate Salt of Compound 1) | of L-tartrate Salt of |
| | | | Compound 1) |
| 8.10 | Citric acid | Clear solution | Clear solution |
| 8.11 | Fumaric acid | + (Crystalline Form A of | + (Crystalline Form B |
| | | Fumarate Salt of Compound | of Fumarate Salt of |
| | | 1 with low crystallinity) | Compound 1) |

Notes:
“+”: Salt hits
“−”: Free form, counter ions, or physical mixtures

TABLE 16
Salt screening results (anti-solvent addition)

| | | | B |
|---|---|---|---|
| | | | Ethanol/ |
| Example | Counter | A | water |
| No. | ions | Acetone | (95:5, v/v) |
| 8.12 | Hydrochloric | 200 μL clear solution→add | 500 μL clear |
| | acid | 600 μL ethyl acetate→thin | solution→add |
| | | suspension→XRPD (AF) | 1500 μL ethyl |
| | | 300 μL clear solution→add | acetate→Hazy |
| | | 300 μL Heptane→gel→dried | solution |
| | | nitrogen→add | |
| | | 300 μL ethyl | |
| | | acetate→gel→suspen- | |
| | | sion→XRPD (AF) | |
| 8.13 | L-malic acid | // | 500 μL clear |
| | | | solution→add |
| | | | 1500 μL ethyl |
| | | | acetate→Clear |
| | | | solution |
| 8.14 | Citric acid | 300 μL clear solution→add | 500 μL clear |
| | | 300 μL ethyl acetate→gel | solution→add |
| | | 200 μL clear solution→add | 1500 μL ethyl |
| | | 200 μL Heptane→gel→ | acetate→Clear |
| | | dried with nitrogen→add | solution |
| | | 100 μL ethyl | |
| | | acetate→gel | |

Notes:
“+”: Salt hits
“−”: Free form, counter ions, or physical mixtures
“AF”: Amorphous form
“//”: Not carried out

### Example 9: Preparation of Salt Candidates

From the salt screening, 4 salts and their polymorphs were identified, including Crystalline Form A of L-malate Salt of Compound 1, Crystalline Form A of L-tartrate Salt of Compound 1, Crystalline Form B of L-tartrate Salt of Compound 1, Crystalline Form A of Fumarate Salt of Compound 1, Crystalline Form B of Fumarate Salt of Compound 1, and Crystalline Form A of Hydrochloride Salt of Compound 1. Among these salts, Crystalline Form A of L-malate Salt of Compound 1 and Crystalline Form A of L-tartrate Salt of Compound 1 showed good physicochemical characteristics including high crystallinity, high melting point, and good stoichiometry; Crystalline Form B of L-tartrate Salt of Compound 1 and Crystalline Form A of Fumarate Salt of Compound 1 are hemi-salts with low crystallinity; Crystalline Form B of Fumarate Salt of Compound 1 has high melting point and reasonable stoichiometry, but the crystallinity is medium; Crystalline Form A of Hydrochloride Salt of Compound 1 is an amorphous form. Therefore, Crystalline Form A of L-malate Salt of Compound 1 and Crystalline Form A of L-tartrate Salt of Compound 1 were selected as salt candidates and scaled up for full evaluation.

Crystalline Form A of L-malate Salt of Compound 1 and Crystalline Form A of L-tartrate Salt of Compound 1 were scaled up to ˜500 mg and evaluated in comparison of free form Crystalline Form A of Compound 1. The procedures for preparing Crystalline Form A of L-malate Salt of Compound t and Crystalline Form A of mL-tartrate Salt of Compound are shown in Table 17 below.

TABLE 17
Preparation of salt candidates

| Lot no. | Procedure and observations |
|---|---|
| Crystalline | 1. 500 mg of free form Crystalline Form A of |
| Form A of | Compound 1 was weighed into a 40 mL vial and |
| L-malate Salt of | 3 mL acetone was added into the vial under stirring |
| Compound 1 | at 50° C. (suspension); |
| | 2. 134 mg L-malic acid (~1.05 equiv.) |
| | was dissolved in 2 mL acetone at |
| | 50° C. Then the L-malic acid |
| | solution was added into the solution slowly (clear |
| | solution); |
| | 3. About 21 mg seeds were added to the solution and |
| | kept stirring at 50° C. for about 2 hours (suspension); |
| | 4. Cooled to 25° C. and kept stirring for about 1 day |
| | (suspension); |
| | 5. The solids were collected by centrifugal |
| | filtration and dried in the vacuum oven |
| | at 40° C. for about 16 hours; |
| | 6. 476 mg Crystalline Form A of L-malate Salt of |
| | Compound 1 was obtained as an off-white solid in a |
| | yield of 72.7%. |
| Crystalline | 1. 500 mg of free form Crystalline Form A of |
| Form A of | Compound 1 and 150 mg of L-tartaric acid (~1.05 |
| L-tartrate Salt of | equiv.) were weighed into a 40 mL vial; |
| Compound 1 | 2. 5 mL acetone was added into the vial |
| | under stirring at 50° C. for about |
| | 10 min (clear solution, and a few of |
| | gel substance stuck on the bottom), then about |
| | 0.5 mL acetone was added into the vial. After stirring |
| | for about 5 min, the solids were precipitated out |
| | (suspension). Then kept stirring at 50° C. for about 2 |
| | hours (suspension); |
| | 3. Cooled to 25° C. and kept stirring for about 1 day |
| | (suspension); |
| | 4. The solids were collected by |
| | centrifugal filtration and dried in |
| | the vacuum oven at 40° C. for about 16 hours |
| | and then at 50° C. for about 24 hours; |
| | 5. About 528 mg Crystalline Form A of L-tartrate Salt |
| | of Compound 1 was obtained as an off-white solid |
| | in a yield of 81.2%. |

### Example 10: Characterization of Salt Candidate Crystalline Form a of L-Malate Salt of Compound 1

### (1) XRPD Data

The XRPD pattern for Crystalline Form A of L-malate Salt of Compound 1 is shown in FIG. 18. The peaks and their relative intensities in the XRPD pattern are shown in Table 18 below. The XRPD pattern for Crystalline Form A of L-malate Salt of Compound 1 suggested that Crystalline Form A of L-malate Salt of Compound 1 is anhydrate of high crystallinity.

TABLE 18
Peaks in the XRPD Pattern for Crystalline
Form A of L-malate Salt of Compound 1

| Peak Position [°2θ] | Relative Intensity (%) |
|---|---|
| 19.349 | 100.0 |
| 22.304 | 59.6 |
| 20.078 | 52.3 |
| 24.489 | 50.1 |
| 22.982 | 46.9 |
| 18.186 | 39.0 |
| 19.083 | 34.5 |
| 6.369 | 32.0 |
| 12.702 | 29.8 |
| 24.937 | 29.0 |
| 28.749 | 26.5 |
| 24.407 | 25.9 |
| 23.772 | 23.5 |
| 23.597 | 20.5 |
| 17.581 | 20.2 |
| 16.263 | 19.7 |
| 31.742 | 17.9 |
| 18.334 | 17.9 |
| 25.587 | 17.3 |
| 3.208 | 17.2 |
| 8.765 | 16.6 |
| 9.529 | 16.4 |
| 20.434 | 16.0 |
| 30.739 | 14.1 |
| 18.878 | 13.2 |
| 26.992 | 13.1 |
| 21.941 | 13.0 |
| 20.948 | 12.3 |
| 36.810 | 12.1 |
| 34.693 | 11.1 |
| 32.704 | 10.5 |
| 21.715 | 10.3 |
| 36.432 | 9.5 |
| 31.492 | 9.4 |
| 27.976 | 8.7 |
| 37.328 | 8.5 |
| 17.975 | 8.4 |
| 38.681 | 8.4 |
| 15.886 | 8.1 |
| 13.678 | 7.9 |
| 32.223 | 7.8 |
| 26.405 | 6.7 |
| 27.514 | 6.4 |
| 31.082 | 5.5 |
| 14.561 | 5.3 |
| 39.380 | 4.6 |
| 35.442 | 4.5 |
| 26.035 | 4.3 |
| 26.739 | 4.2 |
| 35.592 | 4.1 |
| 11.409 | 4.0 |
| 10.200 | 3.6 |
| 8.962 | 3.5 |
| 34.334 | 2.9 |
| 28.307 | 2.8 |
| 35.984 | 2.4 |
| 29.228 | 2.0 |
| 39.705 | 2.0 |
| 33.715 | 1.4 |
| 30.135 | 1.2 |

### (2) TGA and DSC Data

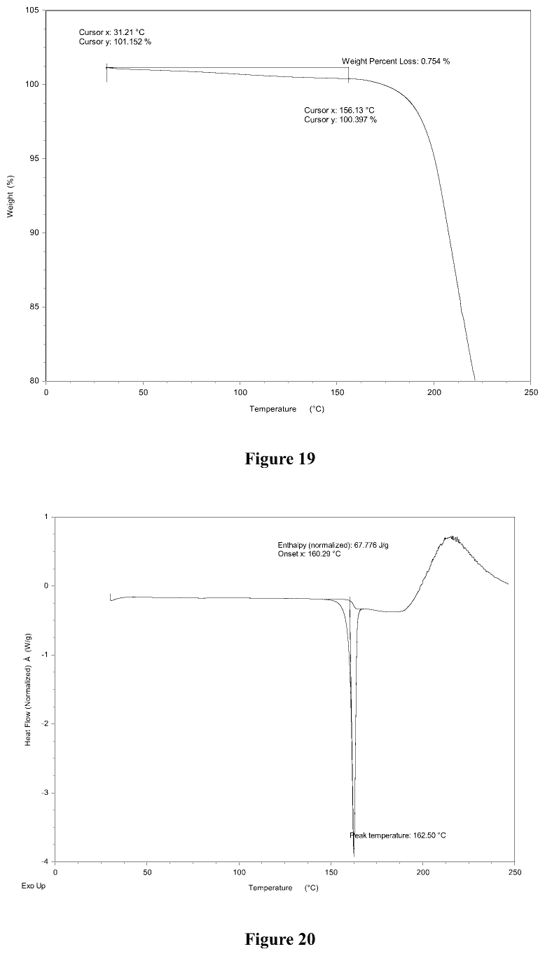

The TGA curve and DSC curve for Crystalline Form A of L-malate Salt of Compound 1 are shown in FIG. 19 and FIG. 20, respectively. As shown in FIG. 19, about 0.8% weight loss was observed between about 31° C. and about 156° C. As shown in FIG. 20, Crystalline Form A of L-malate Salt of Compound 1 has a melting peak of onset about 160.3° C. and enthalpy of about 68 J/g.

### (3) ¹H NMR Data

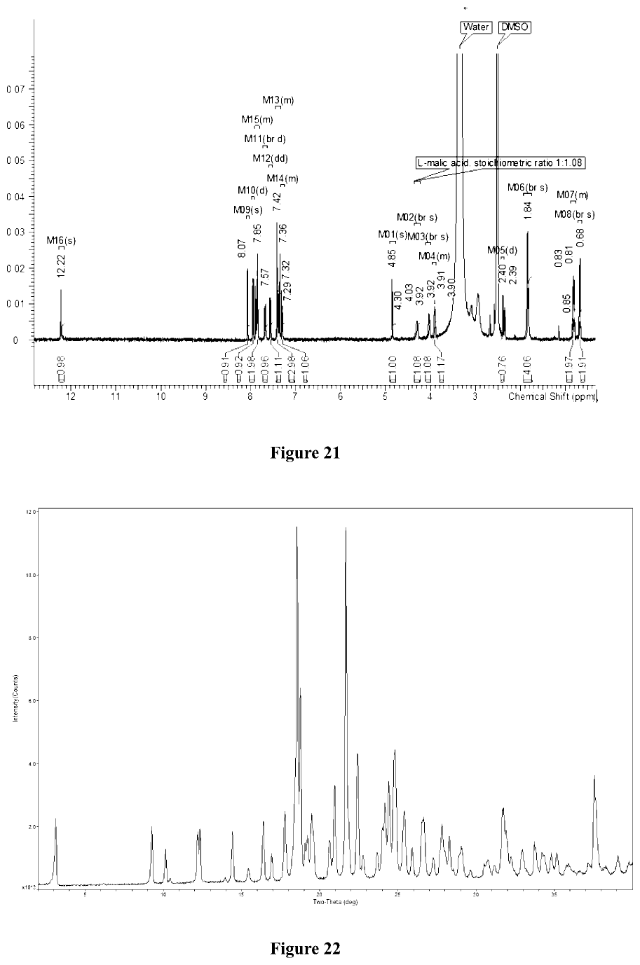

The ¹H NMR spectrum for Crystalline Form A of L-malate Salt of Compound 1 is shown in FIG. 21, suggesting that no residual solvent was detected.

### (4) KF Data

KF analysis showed that Crystalline Form A of L-malate Salt of Compound 1 contained about 0.3% water by weight.

### Example 11: Characterization of Salt Candidate Crystalline Form a of L-Tartrate Salt of Compound 1

### (1) XRPD Data

The XRPD pattern for Crystalline Form A of L-tartrate Salt of Compound 1 is shown in FIG. 22. The peaks and their relative intensities in the XRPD pattern are shown in Table 19 below. The XRPD pattern for Crystalline Form A of L-tartrate Salt of Compound 1 suggested that Crystalline Form A of L-tartrate Salt of Compound 1 is anhydrate of high crystallinity.

TABLE 19
Peaks in the XRPD Pattern for Crystalline
Form A of L-tartrate Salt of Compound 1

| Peak Position [°2θ] | Relative Intensity (%) |
|---|---|
| 18.564 | 100.0 |
| 21.685 | 99.6 |
| 18.774 | 54.3 |
| 24.834 | 35.9 |
| 22.437 | 35.4 |
| 37.586 | 28.0 |
| 24.428 | 27.1 |
| 20.965 | 26.4 |
| 24.193 | 20.4 |
| 17.789 | 19.6 |
| 3.135 | 19.0 |
| 19.490 | 18.6 |
| 25.427 | 18.6 |
| 31.744 | 18.5 |
| 16.413 | 17.0 |
| 9.279 | 16.2 |
| 26.623 | 15.9 |
| 12.360 | 15.2 |
| 27.826 | 14.6 |
| 14.438 | 14.4 |
| 12.221 | 14.3 |
| 24.045 | 14.3 |
| 31.932 | 12.8 |
| 19.234 | 12.0 |
| 28.295 | 11.3 |
| 20.641 | 10.6 |
| 19.080 | 10.3 |
| 10.165 | 9.6 |
| 33.761 | 8.8 |
| 29.071 | 8.6 |
| 25.916 | 8.1 |
| 16.943 | 7.5 |
| 32.967 | 7.2 |
| 23.691 | 7.0 |
| 22.788 | 6.3 |
| 34.232 | 6.1 |
| 35.170 | 6.1 |
| 34.812 | 6.1 |
| 27.271 | 5.1 |
| 32.240 | 5.1 |
| 39.082 | 5.0 |
| 30.774 | 4.7 |
| 15.453 | 3.7 |
| 30.534 | 3.3 |
| 31.183 | 3.0 |
| 37.181 | 2.9 |
| 35.805 | 2.4 |
| 28.554 | 2.0 |
| 29.645 | 1.8 |
| 39.789 | 1.7 |
| 38.258 | 1.7 |
| 10.432 | 1.3 |
| 13.968 | 1.2 |
| 36.619 | 0.7 |

### (2) TGA and DSC Data

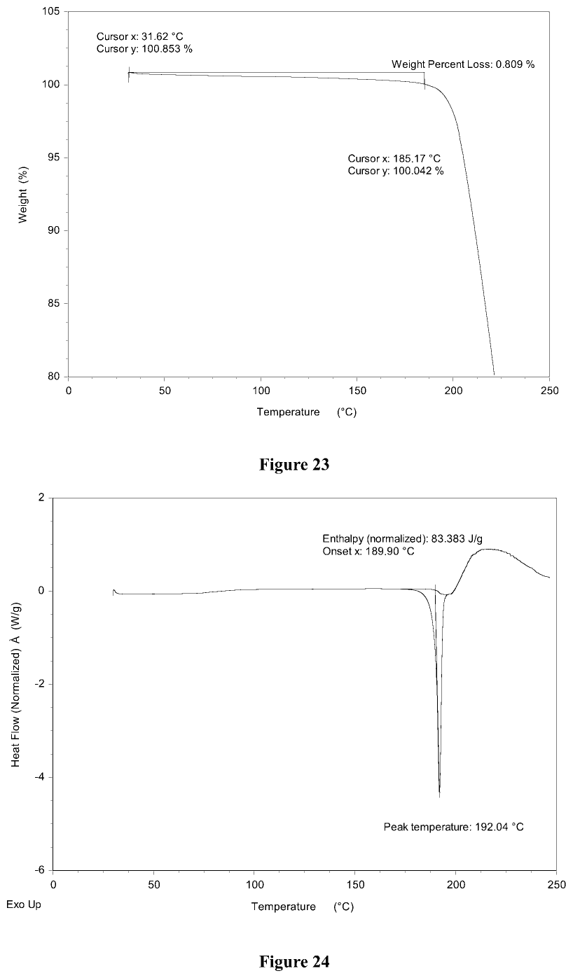

The TGA curve and DSC curve for Crystalline Form A of L-tartrate Salt of Compound 1 are shown in FIG. 23 and FIG. 24, respectively. As shown in FIG. 23, about 0.8% weight loss was observed between about 32° C. and about 185° C. As shown in FIG. 24, Crystalline Form A of L-tartrate Salt of Compound 1 has a melting onset of 189.9° C. and enthalpy of about 83 J/g.

### (3) ¹H NMR Data

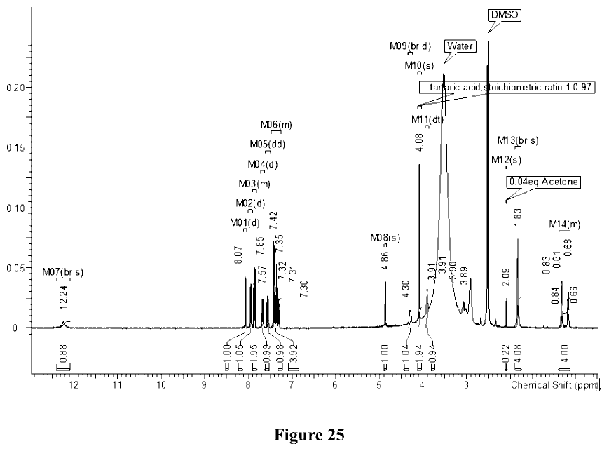

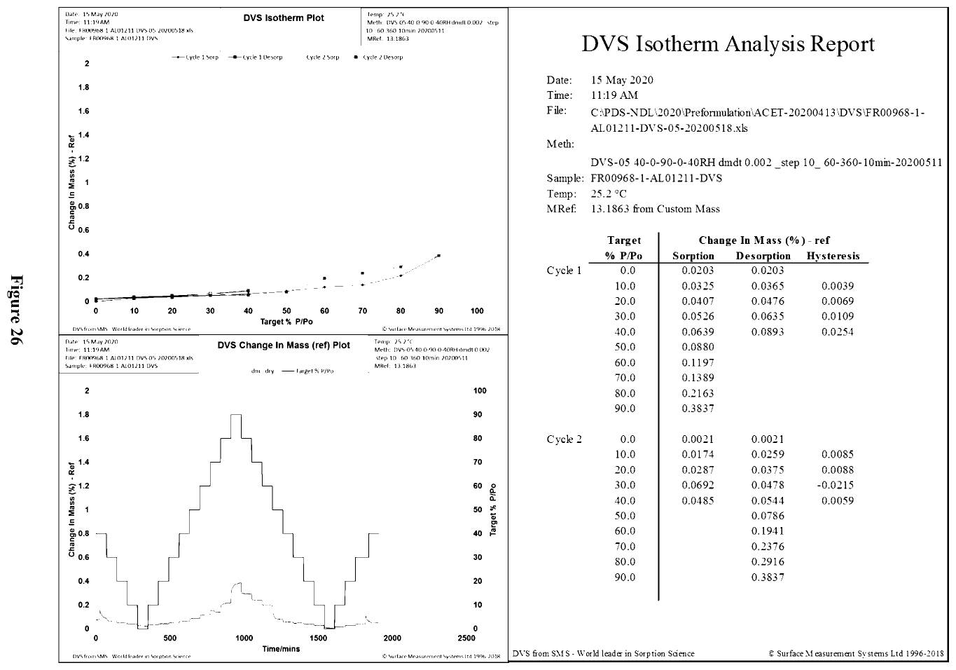

The ¹H NMR spectrum for Crystalline Form A of L-tartrate Salt of Compound 1 is shown in FIG. 25.

### (4) KF Data

KF analysis showed that Crystalline Form A of L-tartrate Salt of Compound 1 contained about 0.6% water by weight.

### Example 12: Evaluation of Salt Candidates

Crystalline Form A of L-malate Salt of Compound 1 and Crystalline Form A of L-tartrate Salt of Compound 1 were scaled up successfully. The scale up batches were the same polymorphs as the screening samples. The two salt candidates were evaluated in comparison with free form Crystalline Form A of Compound 1 for their stability, physicochemical properties, solubility, and hygroscopicity.

### Stability

The purities and appearance of free form Crystalline Form A of Compound 1, Crystalline Form A of L-malate Salt of Compound 1 and Crystalline Form A of L-tartrate Salt of Compound 1 are shown in Table 20 below. As shown in Table 20, the free form Crystalline Form A of Compound 1, the Crystalline Form A of L-malate Salt of Compound 1 and the Crystalline Form A of L-tartrate Salt of Compound 1 have high chemical purity of 99.8%; the free form Crystalline Form A of Compound 1, the Crystalline Form A of L-malate Salt of Compound 1 and the Crystalline Form A of L-tartrate Salt of Compound 1 are all chemically and physically stable at 25° C./92% RH in an open container, at 40° C./75% RH in an open container, and at 60° C. in a tight container for one week.

TABLE 20
Stability: purity and appearance

| | | Physical Form | | | | | |
|---|---|---|---|---|---|---|---|
| | | Free form | | Crystalline | | Crystalline | |
| | | Crystalline | | Form A of L- | | Form A of L- | |
| | | Form A of | | malate Salt of | | tartrate Salt of | |
| | | Compound 1 | | Compound 1 | | Compound 1 | |
| | | Initial purity by HPLC | | | | | |
| Example | | 99.8% | | 99.8% | | 99.8% | |
| No. | Parameter | Purity | CL | Purity | CL | Purity | CL |
| 12.1 | Solid state, 25° C./92% RH, open container, 1 week | | | | | | |
| | Bulk | 99.8% | NC | 99.8% | NC | 99.8% | NC |
| | (HPLC) | | | | | | |
| | Bulk | No form change | | No form change | | No form change | |
| | (XRPD) | | | | | | |
| 12.2 | Solid state, 40° C./75% RH, open container, 1 week | | | | | | |
| | Bulk | 99.8% | NC | 99.8% | NC | 99.8% | NC |
| | (HPLC) | | | | | | |
| | Bulk | No form change | | No form change | | No form change | |
| | (XRPD) | | | | | | |
| 12.3 | Solid state, 60° C., tight container, 1 week | | | | | | |
| | Bulk | 99.8% | NC | 99.8% | NC | 99.8% | NC |
| | (HPLC) | | | | | | |
| | Bulk | No form change | | No form change | | No form change | |
| | (XRPD) | | | | | | |

Notes:
“NC”: No change of color
“CL”: Color

### Crystallinity and Physicochemical Properties

The chemical and physicochemical properties of free form Crystalline Form A of Compound 1, Crystalline Form A of L-malate Salt of Compound 1 and Crystalline Form A of L-tartrate Salt of Compound 1 are shown in Table 21 below.

### Solubility

As shown in Table 21 below, solubility was tested in 4 different pH buffers and 3 bio-relevant media at 37° C. for 2 h and 24 h. As a weak base, Compound 1 showed a typical pH dependent solubility with high solubility in low pH and low solubility in neutral pH. Solubilities of the free form Crystalline Form A of Compound 1 and the two salt candidates were similar in most media except in water and pH 4.5 buffer. The free base and salts showed good solubility in pH 1.2 buffer, SGF and FeSSIF-V1. The higher solubility of the two salt candidates in water is due to lower pH caused by the acidic counter ions. In pH 4.5 buffer, the Crystalline Form A of L-malate Salt of Compound 1 and the Crystalline Form A of L-tartrate Salt of Compound 1 showed about 3-folds and 2-folds higher solubility than that of the free form Crystalline Form A of Compound 1, respectively. However, this solubility increase is not due to the salt but because the two salts disproportionate into amorphous free form in this buffer. An amorphous form generally has higher solubility than a crystalline form. The free form Crystalline Form A of Compound 1 and the salts showed the higher solubility (>2 mg/mL) in FeSSIF-V1 than in buffer of similar pH, suggesting surfactant may be beneficial to solubility improvement.

### Morphic Properties

Hygroscopicity: Hygroscopicity of the free form Crystalline Form A of Compound 1 and the salt candidates was evaluated by dynamic vapor sorption (DVS) test. The free form Crystalline Form A of Compound 1 is slightly hygroscopic, about 0.33% water uptake increased from 40% RH to 90% RH at 25° C., and no form change was observed after the DVS test. The Crystalline Form A of L-malate Salt of Compound 1 is slightly hygroscopic, about 0.59% water uptake increased from 40% RH to 90% RH at 25° C., and no form change was observed after the DVS test. The Crystalline Form A of L-tartrate Salt of Compound 1 is slightly hygroscopic, about 0.39% water uptake increased from 40% RH to 90% RH at 25° C., and no form change was observed after the DVS test.

TABLE 21
Chemical and physicochemical properties of free form Crystalline
Form A of Compound 1, Crystalline Form A of L-malate Salt of Compound
1 and Crystalline Form A of L-tartrate Salt of Compound 1

| | Physical form | | |
|---|---|---|---|
| | Free form | Crystalline Form A | Crystalline Form A |
| | Crystalline Form A | of L-malate Salt of | of L-tartrate Salt of |
| Parameter | of Compound 1 | Compound 1 | Compound 1 |

Initial chemical purity by HPLC

| | | | |
|---|---|---|---|
| Purity [area %] | 99.8% | 99.8% | 99.8% |

Stoichiometry by ¹H-NMR or IC

| | | |
|---|---|---|
| // | 1:1.08 | 1:0.97 |

Residual solvent(s) by ¹H-NMR

| | | | |
|---|---|---|---|
| Weight (%) | No residual solvent | No residual solvent | 0.04 equiv. acetone |
| | | | (0.35% by weight) |

Water content by Karl Fisher

| | | | |
|---|---|---|---|
| Weight (%) | 0.12% | 0.27% | 0.61% |

Crystallinity by XRPD

| | | | |
|---|---|---|---|
| High/medium/low | High | High | High |

DSC, heating rate [10° C./min]

| | | | |
|---|---|---|---|
| Melting | 206.9 | 160.3 | 189.9 |
| onset (° C.) | | | |
| Melting | 97 | 68 | 83 |
| enthalpy | | | |
| (J/g) | | | |

Thermogravimetry, heating rate [10° C./min]

| | | | |
|---|---|---|---|
| Weight | 0.6% @200° C. | 0.8% @156° C. | 0.8% @185° C. |
| loss in (%) | | | |
| @(° C.) | | | |

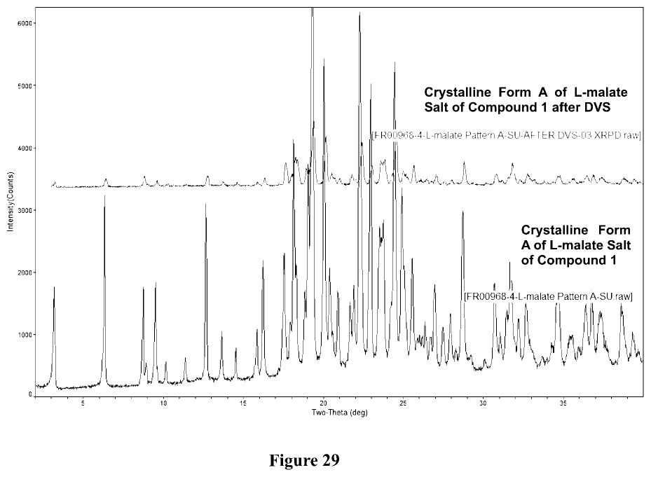

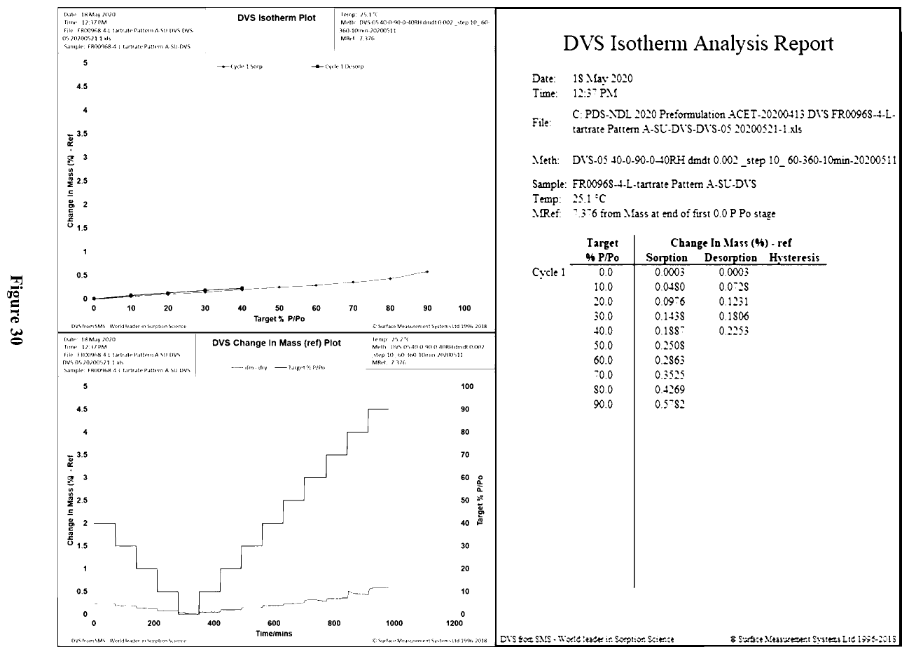

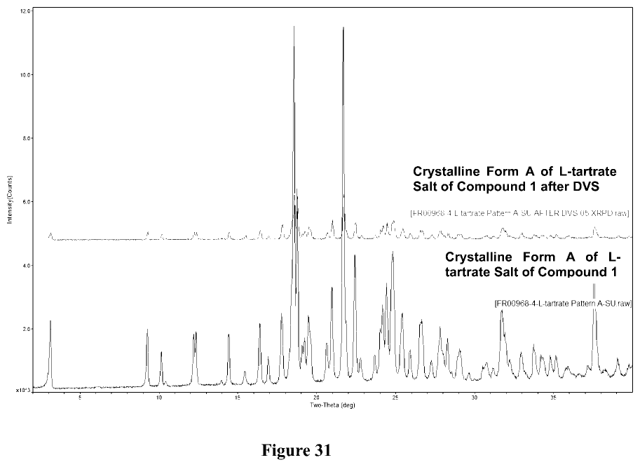

Hygroscopicity by DVS at 25° C. (method: 40-0-90-0-40% RH) dm/dt = 0.002%

| | (FIG. 26) | | (FIG. 28) | | (FIG. 30) | |
|---|---|---|---|---|---|---|
| | Sorp. | Desorp. | Sorp. | Desorp. | Sorp. | Desorp. |
| | (%) | (%) | (%) | (%) | (%) | (%) |
| 0% | 0.02 | 0.02 | 0.17 | 0.17 | 0.00 | 0.00 |
| 10% | 0.03 | 0.04 | 0.22 | 0.22 | 0.04 | 0.07 |
| 20% | 0.04 | 0.05 | 0.26 | 0.26 | 0.09 | 0.12 |
| 30% | 0.05 | 0.06 | 0.30 | 0.30 | 0.14 | 0.18 |
| 40% | 0.06 | 0.09 | 0.33 | 0.33 | 0.18 | 0.22 |
| 50% | 0.09 | // | 0.36 | // | 0.25 | // |
| 60% | 0.12 | // | 0.40 | // | 0.28 | // |
| 70% | 0.14 | // | 0.48 | // | 0.35 | // |
| 80% | 0.22 | // | 0.56 | // | 0.42 | // |
| 90% | 0.39 | // | 0.92 | // | 0.57 | // |
| XRPD | No form change | | No form change | | No form change | |
| after | (FIG. 27) | | (FIG. 29) | | (FIG. 31) | |
| DVS | | | | | | |
| test | | | | | | |

Solubility at 37° C., target concentration 20 mg/10 mL,
equilibration for 2 and 24 hours, LOQ: 0.005 mg/mL

| | | | | | | | |
|---|---|---|---|---|---|---|---|
| Example | | Solubility | XRPD | Solubility | XRPD | Solubility | XRPD |
| No. | Media | (pH) | (24 h) | (pH) | (24 h) | (pH) | (24 h) |

| | |
|---|---|
| 12.4 | pH 1.2, 0.1N HCl |

| 2 hours | 0.96 | No | 0.84 | No | 0.82 | No |
|---|---|---|---|---|---|---|
| | (1.31) | sufficient | (1.23) | sufficient | (1.28) | sufficient |
| 24 hours | 0.97 | solids | 1.06 | solids | 1.09 | solids |
| | (1.05) | | (1.02) | | (1.17) | |

| | |
|---|---|
| 12.5 | pH 4.5, 50 mM acetate buffer |

| 2 hours | 0.23 | No form | 0.60 | No | 0.48 | No |
|---|---|---|---|---|---|---|
| | (4.63) | change | (4.51) | sufficient | (4.44) | sufficient |
| 24 hours | 0.21 | | 0.65 | solids | 0.48 | solids |
| | (4.49) | | (4.49) | | (4.49) | |

| | |
|---|---|
| 12.6 | pH 6.8, 50 mM phosphate buffer |

| 2 hours | 0.01 | No form | 0.02 | Amorphous | 0.02 | Amorphous |
|---|---|---|---|---|---|---|
| | (6.88) | change | (6.68) | form | (6.61) | form |
| 24 hours | 0.005 | | 0.02 | | 0.02 | |
| | (6.85) | | (6.60) | | (6.60) | |

| | |
|---|---|
| 12.7 | Water |

| 2 hours | <LOQ | No form | 1.13 | No | 0.86 | No |
|---|---|---|---|---|---|---|
| | (6.28) | change | (4.17) | sufficient | (3.45) | sufficient |
| 24 hours | <LOQ | | 1.32 | solids | 0.84 | solids |
| | (7.84) | | (3.99) | | (3.60) | |

| | |
|---|---|
| 12.8 | SGF, pH 2.0 |

| 2 hours | 1.16 | No | 1.14 | No | 1.11 | No |
|---|---|---|---|---|---|---|
| | (2.33) | sufficient | (2.29) | sufficient | (2.20) | sufficient |
| 24 hours | 1.06 | solids | 0.95 | solids | 0.96 | solids |
| | (2.08) | | (2.13) | | (2.22) | |

| | |
|---|---|
| 12.9 | FeSSIF-V1, pH 5.0 |

| 2 hours | 1.97 | Clear | 1.57 | Clear | 1.65 | Clear |
|---|---|---|---|---|---|---|
| | (5.18) | solution | (5.09) | solution | (5.02) | solution |
| 24 hours | >2.00 | | >2.00 | | >2.00 | |
| | (5.06) | | (4.94) | | (4.94) | |

| | |
|---|---|
| 12.10 | FaSSIF-V1, pH 6.5 |

| 2 hours | 0.03 | No form | 0.04 | No | 0.04 | No |
|---|---|---|---|---|---|---|
| | (6.54) | change | (6.30) | sufficient | (6.34) | sufficient |
| 24 hours | 0.02 | | 0.04 | solids | 0.04 | solids |
| | (6.51) | | (6.22) | | (6.27) | |

In summary, for oral applications, the free form Crystalline Form A of Compound 1, the Crystalline Form A of L-malate Salt of Compound 1 and Crystalline Form A of L-tartrate Salt of Compound 1 showed good crystallinity, high melting point and reasonable stoichiometry. They also exhibited satisfactory stability, slight hygroscopicity and comparable solubility except in pH 4.5 buffer. PK study showed the free form and the two salts have similar PK.

### Example 13: Biological Activity Study

In this study, the inventors developed a reliable method to monitor GCS catalytic activity using the fluorescent substrate, C6-NBD-ceramide. The reaction product, C6-NBD-glucosylceramide, could then be detected and quantified by LC-MS/MS. The inventors used this method to test and validate the effect of various GCS inhibitors on GCS enzyme activity from different species.

The MDCK, K-562, and L6 cell lines all endogenously express GCS enzyme and were used to test the effect of various GCS inhibitors on enzyme originating from different species. The GCS enzyme activity was detected by using the fluorescent substrate, C6-NBD-ceramide, and monitoring the formation of the fluorescent reaction product, C6-NBD-glucosylceramide. First, the inventors used cell lysate derived from a canine kidney cell line (MDCK, ATCC Cat #CCL-34™) as the source of GCS and tested the inhibitory activity of several control and test compounds. The inventors then used the human leukemia K-562 cell line (ATCC Cat #CRL-1458™) to test the effect of these GCS inhibitors on intact living cells. Finally, a cell lysate derived from a rat skeletal muscle cell line, L6 (ATCC Cat #CCL-243™), was used to test the effect of these GCS inhibitors on rodent GCS enzyme.

The information of test compounds and control compounds are listed as follows.

TABLE 22
Test Compounds

| Compound | | Molecular |
|---|---|---|
| Name | Chemical Structure | Weight |
| Crystalline Form A of Compound 1 | 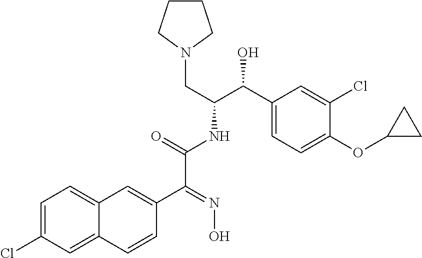 [SMILES: O=C(N[C@H](CN1CCCC1)[C@H](O)c1ccc(OC2CC2)c(Cl)c1)/C(=N/O)c1ccc2cc(Cl)ccc2c1] | 542.45 |
| Geometric isomer of Compound 1 | 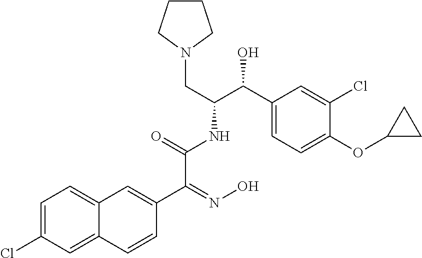 [SMILES: O=C(N[C@H](CN1CCCC1)[C@H](O)c1ccc(OC2CC2)c(Cl)c1)/C(=N\O)c1ccc2cc(Cl)ccc2c1] | 542.45 |

TABLE 23
Control Compounds

| Compound | | Molecular |
|---|---|---|
| Name | Chemical Structure | Weight |
| Eliglustat | 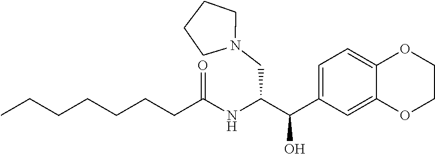 [SMILES: CCCCCCCC(=O)N[C@H](CN1CCCC1)[C@H](O)c1ccc2c(c1)OCCO2] | 404.54 |
| Venglustat | 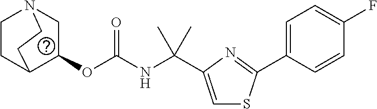 [SMILES: CC(C)(NC(=O)O[C@@H]1CN2CCC1CC2)c1csc(-c2ccc(F)cc2)n1] | 389.49 |

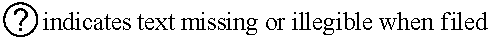

### GCS Activity Test by Enzymatic Assay

GCS activity was evaluated using a GCS enzyme assay derived from MDCK or L6 cell lysates.

MDCK or L6 cell lysates were prepared using M-PER in the presence of a protease inhibitor cocktail. Each 100 mm dish (100% confluent) was lysed with 250 μl of M-PER containing protease inhibitors. Protein concentration was determined using Pierce BCA protein assay kit.

Sixty micrograms of MDCK lysate was incubated with various concentrations of compounds (0.00001 μM-1 μM) in 100 mM Tris buffer (pH 7.5) containing 10 mM MgCl₂, 1 mM dithiothreitol, 1 mM EGTA, 2 mM NAD, 100 μM UDP-glucose, 10 μM C6-NBD-ceramide with 35 μM dioleoylphosphatidylcholine/5 μM sulfatide in a final reaction volume of 100 μl at 37° C. for 1 hour. The final concentration of DMSO was 0.1% in both compound treated and DMSO only treated samples. Each individual reaction was terminated with 11 volumes of methanol:acetonitrile (6:5; containing 200 ng/ml Tolbutamide as internal control). After sufficient mixing, reactions were centrifuged at 2000 rpm for 10 mins, supernatant was injected into the LC-MS/MS for analysis.

For the assay with L6 cell lysate, different amounts of L6 cell lysate were used.

### GCS Activity Test by Cell-Based Assay

The GCS activity was evaluated with a GCS cell-based assay using K-562 cells.

K-562 cells were seeded into 12-well plates (3×10⁵ cells/well, in 1 ml) in RPMI-1640 medium with 5% FBS and incubated at 37° C. for 24 h. These cells were incubated with various concentrations of compounds (0.00001 μM-1 μM) for 4 hours. 100 μl of [RPMI-1640+5% FBS+11% BSA+110 μM C6-NBD-Ceramide] containing compounds or DMSO was then added to the cells, mixed well, and incubated for 0.5 hours at 37° C. After incubation, the cells were centrifuged, washed twice with ice-cold DPBS (pH 7.4), and re-suspended with 50 μl cold DPBS+1% TritonX-100. The mixture was sonicated for 15 mins. A small aliquot (2.5 μl) was used for protein concentration determination. 11 volumes of methanol:acetonitrile (6:5; containing 200 ng/ml Tolbutamide as internal control) was then added to each sample. After sufficient mixing, the samples were centrifuged at 2000 rpm for 10 mins and the resulting supernatant was injected into the LC-MS/MS for analysis.

The quantitative analysis of C6-NBD-glucosylceramide was performed on a Shimadzu ultra-fast liquid chromatography coupled with API 4000 mass spectrometer. Analyst 1.5 software packages were used to control the LC-MS/MS system, as well as for data acquisition and processing.

The C6-NBD-glucosylceramide reading was normalized first by dividing the peak area of C6-NBD-glucosylceramide with the peak area of the internal standard, and then the amount was calculated according to the standard curve generated in the same assay run. For the cell based assay results, the amount of C6-NBD-glucosylceramide was also normalized by the protein concentration of the cell samples.

IC₅₀ values were generated from sigmoidal dose-response (variable slope) curves with GraphPad Prism software (GraphPad Software, Inc., San Diego, CA) using the percent inhibition of C6-NBD-glucosylceramide accumulation relative to DMSO control.

GCS Inhibitors Effect Test with MDCK Cell Lysate

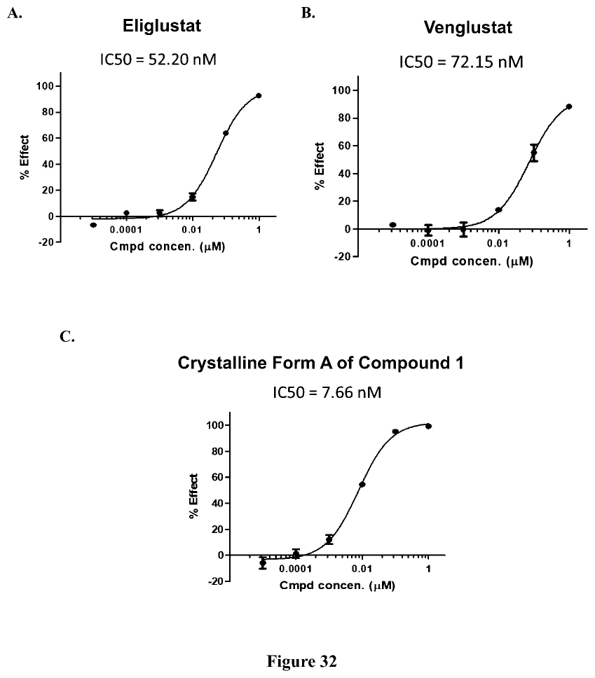

The effect of GCS inhibitors was tested using MDCK cell lysate as the source of GCS enzyme. The compounds Eliglustat and Venglustat were used as controls. Crystalline Form A of Compound 1 was tested. The results of GCS inhibitors effect test with MDCK cell lysate are shown in FIG. 32A (Eliglustat), FIG. 32B (Venglustat) and FIG. 32C (Crystalline Form A of Compound 1), respectively, and were also summarized in Table 24 below. The results showed that the test compound Crystalline Form A of Compound 1 had better inhibitory effect on GCS activity compared to controls.

TABLE 24
Summary of GCS Inhibitors Effect on MDCK Cell Lysate

| Compound | IC₅₀ (nM) |
|---|---|
| Eliglustat | 52.20 |
| Venglustat | 72.15 |
| Crystalline Form A of Compound | 7.66 |
| 1 | |

GCS Inhibitors Effect Test with Live K-562 Cells

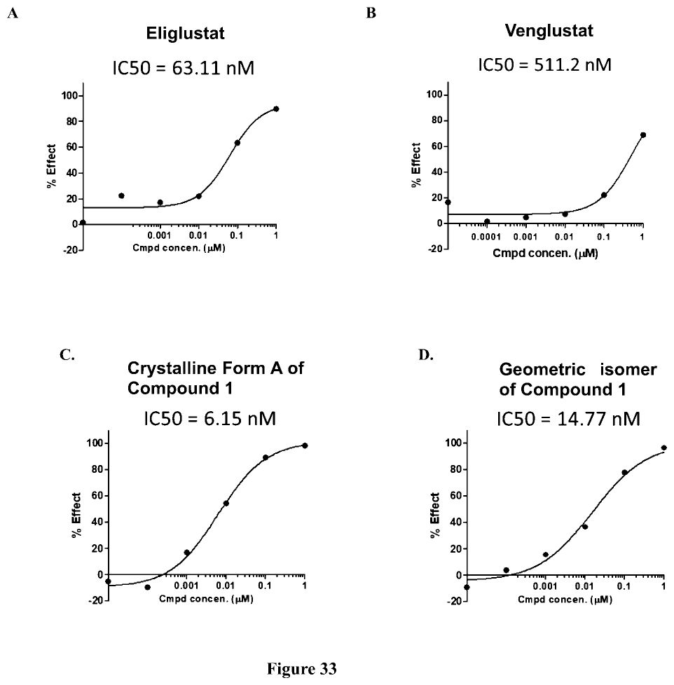

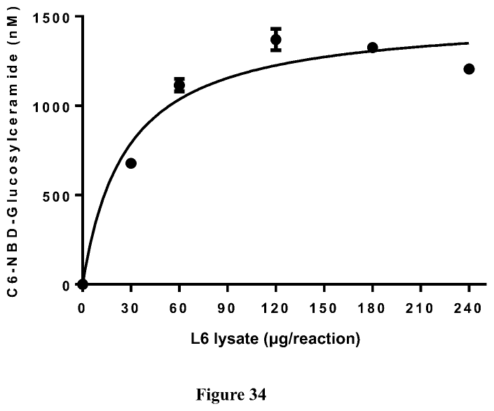

The effect of GCS inhibitors was tested using live K-562 cells as the source of GCS enzyme. The compounds Eliglustat and Venglustat were used as controls. The compounds, Crystalline Form A of Compound 1 and a geometric isomer of Compound 1, were tested. The results of GCS inhibitors effect test with live K-562 cells are shown in FIG. 33A (Eliglustat), FIG. 33B (Venglustat), FIG. 33C (Crystalline Form A of Compound 1) and FIG. 33D (geometric isomer of Compound 1), respectively, and were also summarized in Table 25 below. The results showed that the test compounds (i.e., Crystalline Form A of Compound 1 and geometric isomer of Compound 1) inhibited GCS activity and were more potent than the control compounds Eliglustat and Venglustat.

TABLE 25
Summary of GCS Inhibitors Effect on Live K-562 Cell

| Compound | IC₅₀ (nM) |
|---|---|
| Eliglustat | 63.11 |
| Venglustat | 511.2 |
| Crystalline Form A of Compound 1 | 6.15 |
| Geometric isomer of Compound 1 | 14.77 |

GCS Inhibitors Effect Test with L6 Cell Lysate

To confirm the inhibitory activity of test compounds on GCS from different species, cells from rat were used. First, the presence of GCS activity was verified using various amounts of L6 cell lysate (FIG. 34, Table 26). Next, the inhibitory effect of Crystalline Form A of Compound 1 on GCS activity from L6 cell lysate was tested. The Crystalline Form A of Compound 1 showed potent inhibitory effect on GCS derived from rat skeletal muscle (Table 27 and Table 28). The level of GCS inhibition by Crystalline Form A of Compound 1 was >98% when 30 μg of cell lysate was used, which is consistent with Crystalline Form A of Compound 1 having potent inhibitory activity toward rat GCS enzyme.

TABLE 26
Summary of GCS Activity of L6 Cell Lysate

| | | | | C6-NBD- | |
|---|---|---|---|---|---|
| | | | | Glucosylceramide | |
| Lysate/ | | C6-NBD-Ceramide | | Concentration (nM) | |
| reaction | Conditions | N = 1 | N = 2 | N = 1 | N = 2 |
| 30 μg L6 | 0.1% DMSO | 4.49 | 4.49 | 670 | 687 |
| 60 μg L6 | | 4.47 | 4.70 | 1080 | 1150 |
| 120 μg L6 | | 5.00 | 4.80 | 1430 | 1310 |
| 180 μg L6 | | 4.30 | 4.22 | 1330 | 1320 |
| 240 μg L6 | | 3.76 | 3.76 | 1220 | 1190 |
| 60 μg MDCK | 0.1% DMSO | 3.96 | 4.23 | 959 | 1020 |

TABLE 27
Summary of GCS Inhibitors Effect on L6 Cell Lysate - Control Compounds

| | | | C6-NBD-Ceramide | | | | | |
|---|---|---|---|---|---|---|---|---|
| | | | (Normalized to | | C6-NBD-Glucosylceramide | | | |
| Lysate/ | | | internal control) | | Concentration (nM) | | Inhibition (%) | |
| reaction | | Conditions | N = 1 | N = 2 | N = 1 | N = 2 | N = 1 | N = 2 |
| 30 | μg | Eliglustat 1 μM | 5.98 | 6.37 | 31.2 | 51.0 | 95.40% | 92.48% |
| 60 | μg | | 6.47 | 5.46 | 50.2 | 44.1 | 95.50% | 96.04% |
| 120 | μg | | 5.82 | 6.23 | 48.4 | 57.9 | 96.47% | 95.77% |
| 180 | μg | | 5.60 | 5.41 | 63.6 | 63.3 | 95.20% | 95.22% |
| 240 | μg | | 4.96 | 5.12 | 56.2 | 57.7 | 95.34% | 95.21% |
| 60 | μg MDCK | Eliglustat 1 μM | 4.68 | 4.99 | 49.8 | 59.3 | 94.97% | 94.01% |

TABLE 28
Summary of GCS Inhibitors Effect on L6 Cell Lysate - Test Compounds

| | | | C6-NBD-Ceramide | | C6-NBD- | | | |
|---|---|---|---|---|---|---|---|---|
| | | | (Normalized to | | Glucosylceramide | | | |
| Lysate/ | | | internal control) | | Concentration (nM) | | Inhibition (%) | |
| reaction | | Conditions | N = 1 | N = 2 | N = 1 | N = 2 | N = 1 | N = 2 |
| 30 | μg | Crystalline Form | 6.69 | 6.31 | 9.73 | 10.5 | 98.57% | 98.45% |
| 60 | μg | A of Compound 1 | 5.87 | 6.17 | 22.5 | 26.3 | 97.98% | 97.64% |
| 120 | μg | 0.1 μM | 6.09 | 6.11 | 45.8 | 44.7 | 96.66% | 96.74% |
| 180 | μg | | 5.15 | 5.12 | 54.7 | 59.0 | 95.87% | 95.55% |
| 240 | μg | | 5.72 | 5.61 | 76.9 | 78.2 | 93.62% | 93.51% |
| 60 | μg MDCK | Crystalline Form | 5.04 | 4.88 | 33.1 | 32.5 | 96.65% | 96.72% |
| | | A of Compound 1 | | | | | | |
| | | 0.1 μM | | | | | | |

These results showed that, compared with control compounds (Eliglustat and Venglustat), the test compounds (Crystalline Form A of Compound 1 and geometric isomer of Compound 1) had more potent inhibitory activity for GCS. These test compounds also showed similar inhibitory activity on GCS enzyme derived from different organisms including rat, dog, and human.

What is claimed is:

1. A Crystalline Form A of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide, wherein the Crystalline Form A is characterized by an X-ray powder diffraction (XRPD) pattern comprising one or more peaks selected from the group consisting of about 18.883°, about 10.889°, and about 21.309° 2θ.

2. The Crystalline Form A of claim 1, characterized by an XRPD pattern comprising all the peaks selected from the group consisting of about 18.883°, about 10.889°, and about 21.309° 2θ.

3. The Crystalline Form A of claim 1, characterized by an XRPD pattern further comprising one or more peaks selected from the group consisting of about 14.974°, about 24.160°, about 18.663°, about 17.109°, about 7.518°, about 20.950°, about 19.602°, about 27.784°, and about 28.281° 2θ.

4. The Crystalline Form A of claim 1, characterized by an XRPD pattern further comprising two or more peaks selected from the group consisting of about 14.974°, about 24.160°, about 18.663°, about 17.109°, about 7.518°, about 20.950°, about 19.602°, about 27.784°, and about 28.281° 2θ.

5. The Crystalline Form A of claim 1, characterized by an XRPD pattern further comprising three or more peaks selected from the group consisting of about 14.974°, about 24.160°, about 18.663°, about 17.109°, about 7.518°, about 20.950°, about 19.602°, about 27.784°, and about 28.281° 2θ.

6. The Crystalline Form A of claim 1, characterized by an XRPD pattern further comprising all the peaks selected from the group consisting of about 14.974°, about 24.160°, about 18.663°, about 17.109°, about 7.518°, about 20.950°, about 19.602°, about 27.784°, and about 28.281° 2θ.

7. The Crystalline Form A of claim 3, characterized by an XRPD pattern further comprising one or more peaks selected from the group consisting of about 17.494°, about 14.743°, about 22.386°, about 31.979°, about 21.759°, about 25.017°, about 30.623°, about 10.301°, about 26.760°, about 21.536°, about 19.337°, about 30.103°, about 33.310°, about 31.467°, about 23.309°, about 25.781°, and about 27.362° 2θ.

8. The Crystalline Form A of claim 3, characterized by an XRPD pattern further comprising two or more peaks selected from the group consisting of about 17.494°, about 14.743°, about 22.386°, about 31.979°, about 21.759°, about 25.017°, about 30.623°, about 10.301°, about 26.760°, about 21.536°, about 19.337°, about 30.103°, about 33.310°, about 31.467°, about 23.309°, about 25.781°, and about 27.362° 2θ.

9. The Crystalline Form A of claim 3, characterized by an XRPD pattern further comprising three or more peaks selected from the group consisting of about 17.494°, about 14.743°, about 22.386°, about 31.979°, about 21.759°, about 25.017°, about 30.623°, about 10.301°, about 26.760°, about 21.536°, about 19.337°, about 30.103°, about 33.310°, about 31.467°, about 23.309°, about 25.781°, and about 27.362° 2θ.

10. The Crystalline Form A of claim 3, characterized by an XRPD pattern further comprising all the peaks selected from the group consisting of about 17.494°, about 14.743°, about 22.386°, about 31.979°, about 21.759°, about 25.017°, about 30.623°, about 10.301°, about 26.760°, about 21.536°, about 19.337°, about 30.103°, about 33.310°, about 31.467°, about 23.309°, about 25.781°, and about 27.362° 2θ.

11. The Crystalline Form A of any one of the preceding claims, characterized by an XRPD pattern comprising all the peaks selected from the group consisting of:

| Angle (°2θ) | Angle (°2θ) | Angle (°2θ) | Angle (°2θ) |
|---|---|---|---|
| 18.883 | 14.743 | 23.309 | 28.891 |
| 10.889 | 22.386 | 25.781 | 34.526 |
| 21.309 | 31.979 | 27.362 | 17.884 |
| 14.974 | 21.759 | 35.751 | 13.697 |
| 24.160 | 25.017 | 23.835 | 32.877 |
| 18.663 | 30.623 | 23.601 | 36.134 |
| 17.109 | 10.301 | 37.946 | 11.644 |
| 7.518 | 26.760 | 33.960 | 37.542 |
| 20.950 | 21.536 | 39.178 | 16.364 |
| 19.602 | 19.337 | 10.569 | 14.156 |
| 27.784 | 30.103 | 36.533 | 35.228 |
| 28.281 | 33.310 | 26.283 | |
| 17.494 | 31.467 | 29.254 | |

12. The Crystalline Form A of any one of the preceding claims, wherein the XRPD pattern is substantially as shown in FIG. 1.

13. The Crystalline Form A of any one of the preceding claims, characterized by a differential scanning calorimetry (DSC) curve that comprises an endotherm at about 206.9° C.

14. The Crystalline Form A of claim 13, wherein the DSC curve is substantially as shown in FIG. 3.

15. A Crystalline Form B of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide, wherein the Crystalline Form B is characterized by an XRPD pattern comprising one or more peaks selected from the group consisting of about 6.244°, about 18.679°, and about 12.445° 2θ.

16. The Crystalline Form B of claim 15, characterized by an XRPD pattern comprising all the peaks selected from the group consisting of about 6.244°, about 18.679°, and about 12.445° 2θ.

17. The Crystalline Form B of claim 15, characterized by an XRPD pattern further comprising one or more peaks selected from the group consisting of about 17.239°, about 12.832°, about 23.559°, about 15.252°, about 22.410°, about 9.344°, and about 25.722° 2θ.

18. The Crystalline Form B of claim 15, characterized by an XRPD pattern further comprising two or more peaks selected from the group consisting of about 17.239°, about 12.832°, about 23.559°, about 15.252°, about 22.410°, about 9.344°, and about 25.722° 2θ.

19. The Crystalline Form B of claim 15, characterized by an XRPD pattern further comprising three or more peaks selected from the group consisting of about 17.239°, about 12.832°, about 23.559°, about 15.252°, about 22.410°, about 9.344°, and about 25.722° 2θ.

20. The Crystalline Form B of claim 15, characterized by an XRPD pattern further comprising all the peaks selected from the group consisting of about 17.239°, about 12.832°, about 23.559°, about 15.252°, about 22.410°, about 9.344°, and about 25.722° 2θ.

21. The Crystalline Form B of claim 17, characterized by an XRPD pattern further comprising one or more peaks selected from the group consisting of about 20.354°, about 16.366°, about 24.731°, about 20.581°, about 28.664°, about 27.044°, about 35.758°, about 17.905°, about 16.087°, about 36.194°, about 34.867°, about 24.984°, about 10.802°, about 29.902°, and about 32.584° 2θ.

22. The Crystalline Form B of claim 17, characterized by an XRPD pattern further comprising two or more peaks selected from the group consisting of about 20.354°, about 16.366°, about 24.731°, about 20.581°, about 28.664°, about 27.044°, about 35.758°, about 17.905°, about 16.087°, about 36.194°, about 34.867°, about 24.984°, about 10.802°, about 29.902°, and about 32.584° 2θ.

23. The Crystalline Form B of claim 17, characterized by an XRPD pattern further comprising three or more peaks selected from the group consisting of about 20.354°, about 16.366°, about 24.7310, about 20.5810, about 28.664°, about 27.044°, about 35.758°, about 17.905°, about 16.087°, about 36.194°, about 34.867°, about 24.984°, about 10.802°, about 29.902°, and about 32.584° 2θ.

24. The Crystalline Form B of claim 17, characterized by an XRPD pattern further comprising all the peaks selected from the group consisting of about 20.354°, about 16.366°, about 24.7310, about 20.581°, about 28.664°, about 27.044°, about 35.758°, about 17.905°, about 16.087°, about 36.194°, about 34.867°, about 24.984°, about 10.802°, about 29.902°, and about 32.584° 2θ.

25. The Crystalline Form B of any one of claims 15 to 24, characterized by an XRPD pattern comprising all the peaks selected from the group consisting of:

| Angle (°2θ) | Angle (°2θ) | Angle (°2θ) | Angle (°2θ) |
|---|---|---|---|
| 6.244 | 9.344 | 35.758 | 32.584 |
| 18.679 | 25.722 | 17.905 | 21.279 |
| 12.445 | 20.354 | 16.087 | 26.266 |
| 17.239 | 16.366 | 36.194 | 37.816 |
| 12.832 | 24.731 | 34.867 | 33.789 |
| 23.559 | 20.581 | 24.984 | 19.275 |
| 15.252 | 28.664 | 10.802 | 19.946 |
| 22.410 | 27.044 | 29.902 | 33.118 |

26. The Crystalline Form B of any one of claims 15 to 25, wherein the XRPD pattern is substantially as shown in FIG. 5.

27. The Crystalline Form B of any one of claims 15 to 26, characterized by a DSC curve that comprises an endotherm at about 178.3° C., about 179.7° C., about 196.6° C., about 198.7° C., and/or about 203.7° C.

28. The Crystalline Form B of claim 27, wherein the DSC curve is substantially as shown in FIG. 7.

29. A Crystalline Form C of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide, wherein the Crystalline Form C is characterized by an XRPD pattern comprising one or more peaks selected from the group consisting of about 6.656°, about 19.994°, and about 26.732° 2θ.

30. The Crystalline Form C of claim 29, characterized by an XRPD pattern comprising all the peaks selected from the group consisting of about 6.656°, about 19.994°, and about 26.732° 2θ.

31. The Crystalline Form C of claim 29, characterized by an XRPD pattern further comprising one or more peaks selected from the group consisting of about 18.350°, about 20.753°, about 22.176°, about 15.941°, about 20.450°, about 13.320°, and about 17.687° 2θ.

32. The Crystalline Form C of claim 29, characterized by an XRPD pattern further comprising two or more peaks selected from the group consisting of about 18.350°, about 20.753°, about 22.176°, about 15.941°, about 20.450°, about 13.320°, and about 17.687° 2θ.

33. The Crystalline Form C of claim 29, characterized by an XRPD pattern further comprising three or more peaks selected from the group consisting of about 18.350°, about 20.753°, about 22.176°, about 15.941°, about 20.450°, about 13.320°, and about 17.687° 2θ.

34. The Crystalline Form C of claim 29, characterized by an XRPD pattern further comprising all the peaks selected from the group consisting of about 18.350°, about 20.753°, about 22.176°, about 15.941°, about 20.450°, about 13.320°, and about 17.687° 2θ.

35. The Crystalline Form C of any one of claims 29 to 34, characterized by an XRPD pattern comprising all the peaks selected from the group consisting of:

| Angle (°2θ) | Angle (°2θ) | Angle (°2θ) |
|---|---|---|
| 6.656 | 20.450 | 12.470 |
| 19.994 | 13.320 | 3.724 |
| 26.732 | 17.687 | 25.036 |
| 18.350 | 19.205 | 30.741 |
| 20.753 | 23.302 | 4.454 |
| 22.176 | 9.183 | |
| 15.941 | 8.700 | |

36. The Crystalline Form C of any one of claims 29 to 35, wherein the XRPD pattern is substantially as shown in FIG. 9.

37. The Crystalline Form C of any one of claims 29 to 36, characterized by a DSC curve that is substantially as shown in FIG. 11.

38. A Crystalline Form D of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide, wherein the Crystalline Form D is characterized by an XRPD pattern comprising one or more peaks selected from the group consisting of about 16.956°, about 5.883°, and about 22.181° 2θ.

39. The Crystalline Form D of claim 38, characterized by an XRPD pattern comprising all the peaks selected from the group consisting of about 16.956°, about 5.883°, and about 22.181° 2θ.

40. The Crystalline Form D of claim 38, characterized by an XRPD pattern further comprising one or more peaks selected from the group consisting of about 17.663°, about 13.291°, about 22.909°, about 20.718°, about 26.900°, and about 18.113° 2θ.

41. The Crystalline Form D of claim 38, characterized by an XRPD pattern further comprising two or more peaks selected from the group consisting of about 17.663°, about 13.291°, about 22.909°, about 20.718°, about 26.900°, and about 18.113° 2θ.

42. The Crystalline Form D of claim 38, characterized by an XRPD pattern further comprising three or more peaks selected from the group consisting of about 17.663°, about 13.291°, about 22.909°, about 20.718°, about 26.900°, and about 18.113° 2θ.

43. The Crystalline Form D of claim 38, characterized by an XRPD pattern further comprising all the peaks selected from the group consisting of about 17.663°, about 13.2910, about 22.909°, about 20.718°, about 26.900°, and about 18.113° 2θ.

44. The Crystalline Form D of claim 40, characterized by an XRPD pattern further comprising one or more peaks selected from the group consisting of about 20.205°, about 11.727°, about 21.756°, about 22.617°, about 20.956°, about 29.635°, about 24.990°, about 12.566°, about 10.080°, about 24.078°, about 9.761°, about 19.581°, about 27.280°, about 25.637°, and about 12.754° 2θ.

45. The Crystalline Form D of claim 40, characterized by an XRPD pattern further comprising two or more peaks selected from the group consisting of about 20.205°, about 11.727°, about 21.756°, about 22.617°, about 20.956°, about 29.635°, about 24.990°, about 12.566°, about 10.080°, about 24.078°, about 9.7610, about 19.581°, about 27.280°, about 25.637°, and about 12.754° 2θ.

46. The Crystalline Form D of claim 40, characterized by an XRPD pattern further comprising three or more peaks selected from the group consisting of about 20.205°, about 11.727°, about 21.756°, about 22.617°, about 20.956°, about 29.635°, about 24.990°, about 12.566°, about 10.080°, about 24.078°, about 9.7610, about 19.581°, about 27.280°, about 25.637°, and about 12.754° 2θ.

47. The Crystalline Form D of claim 40, characterized by an XRPD pattern further comprising all the peaks selected from the group consisting of about 20.205°, about 11.727°, about 21.756°, about 22.617°, about 20.956°, about 29.635°, about 24.990°, about 12.566°, about 10.080°, about 24.078°, about 9.761°, about 19.581°, about 27.280°, about 25.637°, and about 12.754° 2θ.

48. The Crystalline Form D of any one of claims 38 to 47, characterized by an XRPD pattern comprising all the peaks selected from the group consisting of:

| Angle (°2θ) | Angle (°2θ) | Angle (°2θ) | Angle (°2θ) |
|---|---|---|---|
| 16.956 | 21.756 | 25.637 | 39.200 |
| 5.883 | 22.617 | 12.754 | 33.344 |
| 22.181 | 20.956 | 14.335 | 18.891 |
| 17.663 | 29.635 | 35.599 | 34.841 |
| 13.291 | 24.990 | 10.411 | 10.830 |
| 22.909 | 12.566 | 30.457 | 28.859 |
| 20.718 | 10.080 | 11.067 | 14.954 |
| 26.900 | 24.078 | 19.175 | 28.254 |
| 18.113 | 9.761 | 34.373 | 31.083 |
| 20.205 | 19.581 | 36.486 | 7.456 |
| 11.727 | 27.280 | 37.185 | |

49. The Crystalline Form D of any one of claims 38 to 48, wherein the XRPD pattern is substantially as shown in FIG. 13.

50. The Crystalline Form D of any one of claims 38 to 49, characterized by a DSC curve that comprises an endotherm at about 207.4° C.

51. The Crystalline Form D of claim 50, wherein the DSC curve is substantially as shown in FIG. 15.

52. A Crystalline Form E of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide, wherein the Crystalline Form E is characterized by an XRPD pattern comprising one or more peaks selected from the group consisting of about 18.089°, about 26.315°, and about 19.775° 2θ.

53. The Crystalline Form E of claim 52, characterized by an XRPD pattern comprising all the peaks selected from the group consisting of about 18.089°, about 26.315°, and about 19.775° 2θ.

54. The Crystalline Form E of claim 52, characterized by an XRPD pattern further comprising one or more peaks selected from the group consisting of about 24.177°, about 20.803°, about 16.018°, about 21.429°, and about 14.608° 2θ.

55. The Crystalline Form E of claim 52, characterized by an XRPD pattern further comprising two or more peaks selected from the group consisting of about 24.177°, about 20.803°, about 16.018°, about 21.429°, and about 14.608° 2θ.

56. The Crystalline Form E of claim 52, characterized by an XRPD pattern further comprising three or more peaks selected from the group consisting of about 24.177°, about 20.803°, about 16.018°, about 21.429°, and about 14.608° 2θ.

57. The Crystalline Form E of claim 52, characterized by an XRPD pattern further comprising all the peaks selected from the group consisting of about 24.177°, about 20.803°, about 16.018°, about 21.429°, and about 14.608° 2θ.

58. The Crystalline Form E of claim 54, characterized by an XRPD pattern further comprising one or more peaks selected from the group consisting of about 21.314°, about 7.794°, about 20.007°, about 8.744°, and about 15.573° 2θ.

59. The Crystalline Form E of claim 54, characterized by an XRPD pattern further comprising two or more peaks selected from the group consisting of about 21.314°, about 7.794°, about 20.007°, about 8.744°, and about 15.573° 2θ.

60. The Crystalline Form E of claim 54, characterized by an XRPD pattern further comprising three or more peaks selected from the group consisting of about 21.314°, about 7.794°, about 20.007°, about 8.744°, and about 15.573° 2θ.

61. The Crystalline Form E of claim 54, characterized by an XRPD pattern further comprising all the peaks selected from the group consisting of about 21.314°, about 7.794°, about 20.007°, about 8.744°, and about 15.573° 2θ.

62. The Crystalline Form E of any one of claims 52 to 61, characterized by an XRPD pattern comprising all the peaks selected from the group consisting of:

| Angle (°2θ) | Angle (°2θ) | Angle (°2θ) | Angle (°2θ) |
|---|---|---|---|
| 18.089 | 18.636 | 27.764 | 29.879 |
| 26.315 | 28.686 | 17.267 | 34.904 |
| 19.775 | 16.43 | 14.962 | 32.851 |
| 24.177 | 25.182 | 18.864 | 35.467 |
| 20.803 | 20.584 | 12.049 | 31.997 |
| 16.018 | 10.856 | 17.085 | 12.878 |
| 21.429 | 22.515 | 7.486 | 11.239 |
| 14.608 | 19.611 | 17.477 | 30.606 |
| 21.314 | 14.233 | 13.613 | 3.691 |
| 7.794 | 22.83 | 36.59 | 34.151 |
| 20.007 | 23.557 | 28.293 | 33.397 |
| 8.744 | 27.166 | 21.743 | |
| 15.573 | 24.647 | 31.524 | |

63. The Crystalline Form E of any one of claims 52 to 62, wherein the XRPD pattern is substantially as shown in FIG. 17.

64. The crystalline form of any one of the preceding claims, wherein the crystalline form is substantially pure.

65. A Crystalline Form A of L-malate Salt of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide characterized by an XRPD pattern comprising one or more peaks selected from the group consisting of about 19.349°, about 22.304°, and about 20.078° 2θ.

66. The Crystalline Form A of L-malate Salt of claim 65, characterized by an XRPD pattern comprising all the peaks selected from the group consisting of about 19.349°, about 22.304°, and about 20.078° 2θ.

67. The Crystalline Form A of L-malate Salt of claim 65, characterized by an XRPD pattern further comprising one or more peaks selected from the group consisting of about 24.489°, about 22.982°, about 18.186°, about 19.083°, about 6.369°, about 12.702°, about 24.937°, about 28.749°, about 24.407°, about 23.772°, about 23.597°, and about 17.581° 2θ.

68. The Crystalline Form A of L-malate Salt of claim 65, characterized by an XRPD pattern further comprising two or more peaks selected from the group consisting of about 24.489°, about 22.982°, about 18.186°, about 19.083°, about 6.369°, about 12.702°, about 24.937°, about 28.749°, about 24.407°, about 23.772°, about 23.597°, and about 17.581° 2θ.

69. The Crystalline Form A of L-malate Salt of claim 65, characterized by an XRPD pattern further comprising three or more peaks selected from the group consisting of about 24.489°, about 22.982°, about 18.186°, about 19.083°, about 6.369°, about 12.702°, about 24.937°, about 28.749°, about 24.407°, about 23.772°, about 23.597°, and about 17.581° 2θ.

70. The Crystalline Form A of L-malate Salt of claim 65, characterized by an XRPD pattern further comprising all the peaks selected from the group consisting of about 24.489°, about 22.982°, about 18.186°, about 19.083°, about 6.369°, about 12.702°, about 24.937°, about 28.749°, about 24.407°, about 23.772°, about 23.597°, and about 17.581° 2θ.

71. The Crystalline Form A of L-malate Salt of claim 67, characterized by an XRPD pattern further comprising one or more peaks selected from the group consisting of about 16.263°, about 31.742°, about 18.334°, about 25.587°, about 3.208°, about 8.765°, about 9.529°, about 20.434°, about 30.739°, about 18.878°, about 26.992°, about 21.941°, about 20.948°, about 36.810°, about 34.693°, about 32.704°, and about 21.715° 2θ.

72. The Crystalline Form A of L-malate Salt of claim 67, characterized by an XRPD pattern further comprising two or more peaks selected from the group consisting of about 16.263°, about 31.742°, about 18.334°, about 25.587°, about 3.208°, about 8.765°, about 9.529°, about 20.434°, about 30.739°, about 18.878°, about 26.992°, about 21.941°, about 20.948°, about 36.810°, about 34.693°, about 32.704°, and about 21.715° 2θ.

73. The Crystalline Form A of L-malate Salt of claim 67, characterized by an XRPD pattern further comprising three or more peaks selected from the group consisting of about 16.263°, about 31.742°, about 18.334°, about 25.587°, about 3.208°, about 8.765°, about 9.529°, about 20.434°, about 30.739°, about 18.878°, about 26.992°, about 21.941°, about 20.948°, about 36.810°, about 34.693°, about 32.704°, and about 21.715° 2θ.

74. The Crystalline Form A of L-malate Salt of claim 67, characterized by an XRPD pattern further comprising all the peaks selected from the group consisting of about 16.263°, about 31.742°, about 18.334°, about 25.587°, about 3.208°, about 8.765°, about 9.529°, about 20.434°, about 30.739°, about 18.878°, about 26.992°, about 21.941°, about 20.948°, about 36.810°, about 34.693°, about 32.704°, and about 21.715° 2θ.

75. The Crystalline Form A of L-malate Salt of any one of claims 65 to 74, characterized by an XRPD pattern comprising all the peaks selected from the group consisting of about 19.349°, about 22.304°, about 20.078°, about 24.489°, about 22.982°, about 18.186°, about 19.083°, about 6.369°, about 12.702°, about 24.937°, about 28.749°, about 24.407°, about 23.772°, about 23.597°, about 17.581°, about 16.263°, about 31.742°, about 18.334°, about 25.587°, about 3.208°, about 8.765°, about 9.529°, about 20.434°, about 30.739°, about 18.878°, about 26.992°, about 21.941°, about 20.948°, about 36.810°, about 34.693°, about 32.704°, about 21.715°, about 36.432°, about 31.492°, about 27.976°, about 37.328°, about 17.975°, about 38.681°, about 15.886°, about 13.678°, about 32.223°, about 26.405°, about 27.514°, about 31.082°, about 14.561°, about 39.38°, about 35.442°, about 26.035°, about 26.739°, about 35.592°, about 11.409°, about 10.2°, about 8.962°, about 34.334°, about 28.307°, about 35.984°, about 29.228°, about 39.705°, about 33.715°, and about 30.135° 2θ.

76. The Crystalline Form A of L-malate Salt of any one of claims 65 to 75, wherein the XRPD pattern is substantially as shown in FIG. 18.

77. The Crystalline Form A of L-malate Salt of any one of claims 65 to 76, characterized by a DSC curve that comprises an endotherm at about 160.3° C.

78. The Crystalline Form A of L-malate Salt of any one of claim 77, wherein the DSC curve is substantially as shown in FIG. 20.

79. A Crystalline Form A of L-tartrate Salt of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide characterized by an XRPD pattern comprising one or more peaks selected from the group consisting of about 18.564°, about 21.685°, and about 18.774° 2θ.

80. The Crystalline Form A of L-tartrate Salt of claim 79, characterized by an XRPD pattern comprising all the peaks selected from the group consisting of about 18.564°, about 21.685°, and about 18.774° 2θ.

81. The Crystalline Form A of L-tartrate Salt of claim 79, characterized by an XRPD pattern further comprising one or more peaks selected from the group consisting of about 24.834°, about 22.437°, about 37.586°, about 24.428°, about 20.965°, and about 24.193° 2θ.

82. The Crystalline Form A of L-tartrate Salt of claim 79, characterized by an XRPD pattern further comprising two or more peaks selected from the group consisting of about 24.834°, about 22.437°, about 37.586°, about 24.428°, about 20.965°, and about 24.193° 2θ.

83. The Crystalline Form A of L-tartrate Salt of claim 79, characterized by an XRPD pattern further comprising three or more peaks selected from the group consisting of about 24.834°, about 22.437°, about 37.586°, about 24.428°, about 20.965°, and about 24.193° 2θ.

84. The Crystalline Form A of L-tartrate Salt of claim 79, characterized by an XRPD pattern further comprising all the peaks selected from the group consisting of about 24.834°, about 22.437°, about 37.586°, about 24.428°, about 20.965°, and about 24.193° 2θ.

85. The Crystalline Form A of L-tartrate Salt of claim 81, characterized by an XRPD pattern further comprising one or more peaks selected from the group consisting of about 17.789°, about 3.135°, about 19.49°, about 25.427°, about 31.744°, about 16.413°, about 9.279°, about 26.623°, about 12.36°, about 27.826°, about 14.438°, about 12.221°, about 24.045°, about 31.932°, about 19.234°, about 28.295°, about 20.641°, and about 19.08° 2θ.

86. The Crystalline Form A of L-tartrate Salt of claim 81, characterized by an XRPD pattern further comprising two or more peaks selected from the group consisting of about 17.789°, about 3.135°, about 19.49°, about 25.427°, about 31.744°, about 16.413°, about 9.279°, about 26.623°, about 12.36°, about 27.826°, about 14.438°, about 12.221°, about 24.045°, about 31.932°, about 19.234°, about 28.295°, about 20.641°, and about 19.08° 2θ.

87. The Crystalline Form A of L-tartrate Salt of claim 81, characterized by an XRPD pattern further comprising three or more peaks selected from the group consisting of about 17.789°, about 3.135°, about 19.49°, about 25.427°, about 31.744°, about 16.413°, about 9.279°, about 26.623°, about 12.36°, about 27.826°, about 14.438°, about 12.221°, about 24.045°, about 31.932°, about 19.234°, about 28.295°, about 20.641°, and about 19.08° 2θ.

88. The Crystalline Form A of L-tartrate Salt of claim 81, characterized by an XRPD pattern further comprising all the peaks selected from the group consisting of about 17.789°, about 3.135°, about 19.49°, about 25.427°, about 31.744°, about 16.413°, about 9.279°, about 26.623°, about 12.36°, about 27.826°, about 14.438°, about 12.221°, about 24.045°, about 31.932°, about 19.234°, about 28.295°, about 20.641°, and about 19.08° 2θ.

89. The Crystalline Form A of L-tartrate Salt of any one of claims 79 to 88, characterized by an XRPD pattern comprising all the peaks selected from the group consisting of about 18.564°, about 21.685°, about 18.774°, about 24.834°, about 22.437°, about 37.586°, about 24.428°, about 20.965°, about 24.193°, about 17.789°, about 3.135°, about 19.49°, about 25.427°, about 31.744°, about 16.413°, about 9.279°, about 26.623°, about 12.36°, about 27.826°, about 14.438°, about 12.221°, about 24.045°, about 31.932°, about 19.234°, about 28.295°, about 20.641°, about 19.08°, about 10.165°, about 33.761°, about 29.071°, about 25.916°, about 16.943°, about 32.967°, about 23.691°, about 22.788°, about 34.232°, about 35.17°, about 34.812°, about 27.271°, about 32.24°, about 39.082°, about 30.774°, about 15.453°, about 30.534°, about 31.183°, about 37.181°, about 35.805°, about 28.554°, about 29.645°, about 39.789°, about 38.258°, about 10.432°, about 13.968°, and about 36.619° 2θ.

90. The Crystalline Form A of L-tartrate Salt of any one of claims 79 to 89, wherein the XRPD pattern is substantially as shown in FIG. 22.

91. The Crystalline Form A of L-tartrate Salt of any one of claims 79 to 90, characterized by a DSC curve that comprises an endotherm at about 189.9° C.

92. The Crystalline Form A of L-tartrate Salt of claim 91, wherein the DSC curve is substantially as shown in FIG. 24.

93. A pharmaceutical composition comprising (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide or a pharmaceutical salt thereof, and a pharmaceutically acceptable excipient, wherein (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide comprises a crystalline form selected from the group consisting of the Crystalline Form A of any one of claims 1 to 14, the Crystalline Form B of any one of claims 15 to 28, the Crystalline Form C of any one of claims 29 to 37, the Crystalline Form D of any one of claims 38 to 51, the Crystalline Form E of any one of claims 52 to 63, the crystalline form of claim 64, the Crystalline Form A of L-malate Salt of any one of claims 65 to 78, and the Crystalline Form A of L-tartrate Salt of any one of claims 79 to 92.

94. The pharmaceutical composition of claim 93, wherein at least about 85%, at least about 90%, at least about 95%, at least about 99%, at least about 99.5%, at least about 99.9%, at least about 99.99% of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide has the Crystalline Form A.

95. The pharmaceutical composition of claim 93, wherein at least about 85%, at least about 90%, at least about 95%, at least about 99%, at least about 99.5%, at least about 99.9%, at least about 99.99% of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide has the Crystalline Form B.

96. The pharmaceutical composition of claim 93, wherein at least about 85%, at least about 90%, at least about 95%, at least about 99%, at least about 99.5%, at least about 99.9%, at least about 99.99% of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide has the Crystalline Form C.

97. The pharmaceutical composition of claim 93, wherein at least about 85%, at least about 90%, at least about 95%, at least about 99%, at least about 99.5%, at least about 99.9%, at least about 99.99% of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide has the Crystalline Form D.

98. The pharmaceutical composition of claim 93, wherein at least about 85%, at least about 90%, at least about 95%, at least about 99%, at least about 99.5%, at least about 99.9%, at least about 99.99% of (E)-N-((1R,2R)-1-(3-chloro-4-cyclopropoxyphenyl)-1-hydroxy-3-(pyrrolidin-1-yl)propan-2-yl)-2-(6-chloronaphthalen-2-yl)-2-(hydroxyimino)acetamide has the Crystalline Form E.

99. The pharmaceutical composition of claim 93, further comprising an additional therapeutic agent.

100. A method for preparing a pharmaceutical composition, comprising mixing the crystalline form of any one of claims 1 to 64, or the Crystalline Form A of L-malate Salt of any one of claims 65 to 78, or the Crystalline Form A of L-tartrate Salt of any one of claims 79 to 92, with a pharmaceutically acceptable excipient.

101. Use of the crystalline form of any one of claims 1 to 64, or the Crystalline Form A of L-malate Salt of any one of claims 65 to 78, or the Crystalline Form A of L-tartrate Salt of any one of claims 69 to 92, or the pharmaceutical composition of any one of claims 93 to 99 in the manufacture of a medicament for:
(a) preventing or treating a disease, disorder or condition that is associated with an abnormal activity/level of the enzyme GCS;
(b) preventing or treating a GCS-mediated disease, disorder or condition;
(c) inhibiting an activity/level of the enzyme GCS; or
(d) non-therapeutically inhibiting an activity/level of the enzyme GCS in vitro.

102. The use of claim 101, wherein the disease, disorder or condition that is associated with an abnormal activity/level of the enzyme GCS, or the GCS-mediated disease, disorder or condition is selected from the group consisting of a glycolipid storage disease (e.g., Tay Sachs, Sandhoffs, GMl gangliosidosis and Fabry diseases); a disease associated with glycolipid accumulation (e.g., Gaucher disease); a disease that cause renal hypertrophy or hyperplasia such as diabetic nephropathy; a disease that cause hyperglycemia or hyperinsulemia; a cancer in which glycolipid synthesis is abnormal; an infectious disease caused by organisms which use cell surface glycolipids as receptors or in which synthesis of glucosylceramide is essential or important; a metabolic disorder such as atherosclerosis, polycystic kidney disease, renal hypertrophy, and diabetes mellitus; obesity; a cancer such as breast cancer, renal adenocarcinoma, brain cancer, neuroblastoma, lung cancer, intestinal cancer, pancreas and prostrate cancer; a neuronal disorder; neuronal injury; an inflammatory disease or disorder (e.g., rheumatoid arthritis, Crohn's disease, asthma and sepsis); pain (e.g., neuropathic pain, inflammatory pain, headache pain, somatic pain, visceral pain, referred pain); a cognitive disorder (e.g., agnosia; amnesia; aphasia; an apraxia; delirium; dementia including AIDS dementia complex, Binswanger's disease, dementia with Lewy Bodies, frontotemporal dementia, mild cognitive impairment, multi-infarct dementia, Pick's disease, semantic dementia, senile dementia, and vascular dementia; and learning disorders including Asperger's syndrome, attention deficit disorder, attention deficit hyperactivity disorder, autism, childhood disintegrative disorder, and Rett syndrome), a neurodegenerative disorder (such as Alzheimer's disease, corticobasal degeneration, Creutzfeldt-Jacob disease, frontotemporal lobar degeneration, Huntington disease, multiple sclerosis, normal pressure hydrocephalus, organic chronic brain syndrome, Parkinson's disease, Pick disease, progressive supranuclear palsy, and senile dementia (Alzheimer type), glomerular disease), and nonalcoholic fatty liver disease (NALD).

103. The use of claim 102, wherein the disease, disorder or condition is Tay Sachs, Sandhoffs, GMl gangliosidosis, Fabry disease, Gaucher disease, or polycystic kidney disease.

104. A method of inhibiting GCS activity, comprising administering to a subject in need thereof an effective amount of the crystalline form of any one of claims 1 to 64, or the Crystalline Form A of L-malate Salt of any one of claims 65 to 78, or the Crystalline Form A of L-tartrate Salt of any one of claims 79 to 92, or the pharmaceutical composition of any one of claims 93 to 99.
